(19) United States
(12) Patent Application Publication
(10) Pub. No.: US 2023/0364262 A1
(43) Pub. Date: Nov. 16, 2023

(54) VIA CYCLOADDITION BILATERALLY FUNCTIONALIZED ANTIBODIES

(71) Applicant: SYNAFFIX B.V., Oss, (NL)

(72) Inventors: Floris Louis VAN DELFT, Nijmegen (NL); Jorin HOOGENBOOM, Wageningen (NL); Sorraya POPAL, Oss (NL); Arnoldus Jacobus VAN SCHAIK, Oss (NL); Laureen DE BEVER, Oss (NL); Remon VAN GEEL, Lithoijen (NL); Maria Antonia WIJDEVEN, Oss (NL); Sander Sebastiaan VAN BERKEL, Oss (NL)

(21) Appl. No.: 17/812,155

(22) Filed: Jul. 12, 2022

Related U.S. Application Data
(63) Continuation of application No. PCT/EP2021/050594, filed on Jan. 13, 2021.

(30) Foreign Application Priority Data
Jan. 13, 2020 (EP) 20151551.7

(51) Int. Cl. A61K 47/69; A61P 35/00

(52) U.S. Cl. CPC A61K 47/6949; A61P 35/00

(57) ABSTRACT

The present invention provides antibody-payload conjugates having a payload-to-antibody ratio of 1. The antibody-payload conjugate having structure (1): 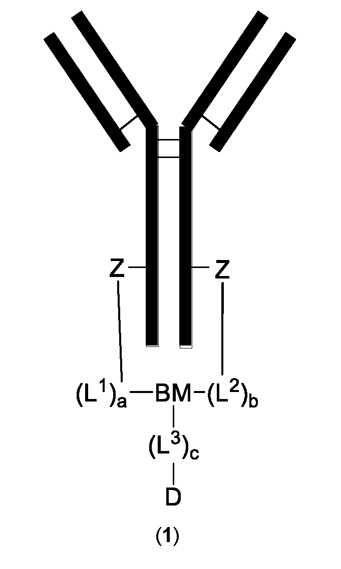 wherein:
a, b and c are each independently 0 or 1;
L¹, L² and L³ are linkers;
D is a payload;
BM is a branching moiety;
Z are connecting groups obtainable by a cycloaddition reaction.

The invention further provides a method for preparing the antibody-payload conjugate according to the invention, an intermediate compound in that preparation method, and medical uses of the antibody-payload conjugate according to the invention.

### FIELD OF THE INVENTION

The present invention relates to the field of bioconjugation, in particular to antibody-conjugates containing a single payload (drug-antibody ratio of 1). More specifically the invention relates to conjugates, compositions and methods suitable for the attachment of a payload to an IgG-type antibody via a cycloaddition reaction. The mono-functionalized antibody conjugates as compounds, compositions, and methods can be useful, for example, in providing novel drugs for targeted delivery of payloads, such as highly potent cytotoxic agents or immunomodulatory agents.

### BACKGROUND OF THE INVENTION

Antibody-drug conjugates (ADC), considered as magic bullets in therapy, are comprised of an antibody to which is attached a pharmaceutical agent. The antibodies (also known as ligands) can be small protein formats (scFv’s, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

An alternative strategy to employ monoclonal antibodies for targeted delivery of a specific protein agent is by genetic fusion of the latter protein to one (or more) of the antibody’s termini, which can be the N-terminus or the C-terminus on the light chain or the heavy chain (or both). In this case, the biologically active protein of interest, e.g. a protein toxin like Pseudomonas exotoxin A (PE38) or an anti-CD3 single chain variable fragment (scFv), is genetically encoded as a fusion to the antibody, possibly but not necessarily via a peptide spacer, so the antibody is expressed as a fusion protein. The peptide spacer may contain a protease-sensitive cleavage site, or not.

A monoclonal antibody may also be genetically modified in the protein sequence itself to modify its structure and thereby introduce (or remove) specific properties. For example, mutations can be made in the antibody Fc-fragment in order to nihilate binding to Fc-gamma receptors, binding to the FcRn receptor or binding to a specific cancer target may be modulated, or antibodies can be engineered to lower the pl and control the clearance rate from circulation. An emerging strategy in cancer treatment involves the use of an antibody that is able to bind to an upregulated tumor-associated antigen (TAA or simply target) as well as to a receptor present on a cancer-destroying immune cell (e.g. a T cell or an NK cell), also known as T cell or NK cell-redirecting antibodies. Although the approach of immune cell redirecting is already more than 30 years old, new technologies are overcoming the limitations of the 1^{st} generation immune cell-redirecting antibodies, especially extending half-life to allow intermittent dosing, reducing immunogenicity and improving the safety profile. Most commonly, T cell-redirecting bispecific antibodies (TRBAs) are generated by genetic swapping of the complement-dependent region (CDR) in one of the arms of the FAB fragment for an antibody fragment that binds tightly to CD3 or CD137 (4-1BB) on a T cell. However, besides these traditional T cell-engaging bispecific antibodies, a wide variety of other molecular architectures, typically IgG-type, have also been developed as for example disclosed in Yu and Wang, J. Cancer Res. Clin. Oncol. 2019, 145, 941-956). Similarly, NK cell recruitment to the tumor microenvironment is also under broad investigation. NK cell engagement is typically based on the insertion into an IgG scaffold of an antibody (fragment) that binds selectively to CD16, CD56, NKp46, or other NK cell specific receptors.

A common strategy in the field of ADCs as well as in the field of immune cell engagement employs nihilation or removal of binding capacity of the antibody to Fc-gamma receptors, which has multiple pharmaceutical implications. The first consequence of removal of binding to Fc-gamma receptors is the reduction of Fc-gamma receptor-mediated uptake of antibodies by e.g. macrophages or megakaryocytes, which may lead to dose-limiting toxicity as for example reported for Kadcyla® (trastuzumab-DM1) and LOP628. Selective deglycosylation of antibodies in vivo affords opportunities to treat patients with antibody-mediated autoimmunity. Removal of high-mannose glycoform in a recombinant therapeutic glycoprotein may be beneficial, since high-mannose glycoforms are known to compromise therapeutic efficacy by aspecific uptake by endogenous mannose receptors and leading to rapid clearance, as for example described by Gorovits and Krinos-Fiorotti, Cancer Immunol. Immunother. 2013, 62, 217-223 and Goetze et al, Glycobiology 2011, 21, 949-959 (both incorporated by reference). In addition, Van de Bovenkamp et al, J. Immunol. 2016, 196, 1435-1441 (incorporated by reference) describe how high mannose glycans can influence immunity. It was described by Reusch and Tejada, Glycobiology 2015, 25, 1325-1334 (incorporated by reference), that inappropriate glycosylation in monoclonal antibodies could contribute to ineffective production from expressed Ig genes. In the field of immune therapy, binding of glycosylated antibodies to Fc-gamma receptors on immune cells may induce systemic activation of the immune system, prior to binding of the antibody to the tumor-associated antigen, leading to cytokine storm (cytokine release syndrome, CRS). Therefore, in order to reduce the risk of CRS, the vast majority of immune cell engagers in the clinic are based on Fc-silenced antibodies, lacking the capacity to bind to Fc-gamma receptors. In addition, various companies in the field of bispecific antibodies are tailoring molecular architectures with defined ratios with regard to target-binding versus immune cell-engaging antibody domains. For example, Roche is developing T cell-engagers based on asymmetric monoclonal antibodies that retain bivalent binding capacity to the TAA (for example CD20 or CEA) by both CDRs, but with an additional anti-CD3 fragment engineered into one of the two heavy chains only (2:1 ratio of target-binding:CD3-binding). Similar strategies can be employed for engagement/activation of T cells with anti-CD137 (4-1BBB) or NK cell-engagement/activation with anti-CD16, CD56, NKp46, or other NK cell specific receptors.

Abrogation of binding to Fc-gamma receptor can be achieved in various ways, for example by specific mutations in the antibody (specifically the Fc-fragment) or by removal of the glycan that is naturally present in the Fc-fragment (CH2 domain, around N297). Glycan removal can be achieved by genetic modification in the Fc-domain, e.g. a N297Q mutation or T299A mutation, or by enzymatic removal of the glycan after recombinant expression of the antibody, using for example PNGase F or an endoglycosidase. For example, endoglycosidase H is known to trim high-mannose and hybrid glycoforms, but not complex type glycans, while endoglycosidase S is able to trim complex type glycans and to some extent hybrid glycan, but not high-mannose forms. Endoglycosidase F2 is able to trim complex glycans (but not hybrid), while endoglycosidase F3 can only trim complex glycans that are also 1,6-fucosylated. Another endoglycosidase, endoglycosidase D is able to hydrolyze Man5 (M5) glycan only. An overview of specific activities of different endoglycosidases is disclosed in Freeze et al. in Curr. Prot. Mol. Biol., 2010, 89:17.13A.1-17, incorporated by reference herein. An additional advantage of deglycosylation of proteins for therapeutic use is the facilitated batch-to-batch consistency and significantly improved homogeneity.

In the field of ADCs, a chemical linker is typically employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment).

Enzyme-based strategies are generally based on the endogenous presence of specific proteases, esterases, glycosidases or others. For example, the majority of ADCs used in oncology utilize the dominant proteases found in a tumour cell lysosome for recognition and cleavage of a specific peptide sequence in the linker. Dubowchik et al., Bioconjug Chem. 2002, 13, 855-69, incorporated by reference, pioneered the discovery of specific dipeptides as an intracellular cleavage mechanism by cathepsins. Other enzymes that are known to be upregulated in the tumour lysozyme or the tumour microenvironment are plasmin, matrix metalloproteases (MMPs), urokinase, and others, all of which may recognize a specific peptide sequence in the ADCs and induce release of payload from the linker by hydrolytic cleavage of one of the peptide bonds. Esterases may also be employed for intracellular release of payload upon hydrolysis of an ester bond, for example it was demonstrated by Barthel et al, J. Med. Chem. 2012, 55, 6595-6607, incorporated by reference, that human carboxylesterase 2 (CES2, hiCE) demonstrated an in vivo antitumor efficacy of a doxorubicin prodrug against CES2-positive xenografts that was better than or equal to that of payload itself. Thirdly, various glycosidases may be employed for selective cleavage of a specific monosaccharide, in particular galactosidase (for removal of galactose) or glucuronidase (for removal of glucuronic acid), as for example illustrated in respectively Torgov et al, Bioconj. Chem. 2005, 16, 717-721 and Jeffrey et al, J. Med. Chem. 2006, 17, 831-840, incorporated by reference. Other endogenous enzymes that may be employed for tumour-specific hydrolytic cleavage of bonds are for example phosphatases or sulfatases.

Besides the use of endogenous enzymes, local concentration enhancement of any enzyme of choice, which may not be naturally abundant, can be achieved by strategies such as systemic administration by intravenous injection, by intratumoural injection or by other methods such as ADEPT (antibody-directed enzyme prodrug therapy).

The acid-sensitivity strategy takes advantage of the lower pH in the endosomal (pH 5-6) 25 and lysosomal (pH 4.8) compartments, as compared to the cytosol of a human cell (pH 7.4), to trigger hydrolysis of an acid labile group within the linker, such as a hydrazone, see for example Ritchie et al, mAbs 2013, 5, 13-21, incorporated by reference. Alternative acid-sensitive linker may also be employed, as for example based on silyl ethers, disclosed in US20180200273.

A third release strategy based on redox mechanisms exploits the higher concentrations of intracellular glutathione than in the plasma. Thus, linkers containing a disulfide bridge release a free thiol group upon reduction by glutathione, which may remain part of the payload or further self-immolate to release the free payload. Alternative reduction mechanisms for release of free payload can be based on the conversion of an (aromatic) nitro group or a (aromatic) azido group into an aniline, which may be part of a payload or part of a self-immolative assembly unit.

A self-immolative assembly unit in an antibody-drug conjugate links a drug unit to the remainder of the conjugate or its drug-linker intermediate. The main function of the self-immolative assembly unit is to conditionally release free drug at the site targeted by the ligand unit. The activatable self-immolative moiety comprises an activatable group and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

The majority of antibody-drug conjugates that have been market-approved or are currently in late-stage clinical trials employ one of the mechanisms described above for release of active drug. For example, Adcetris® is an ADC used for treatment of various hematological tumours and is comprised of a CD30-targeting antibody (ligand), connected to a highly potent tubulin inhibitor MMAE (payload) via a linker that consists of a cathepsin-sensitive fragment connected to a self- immolative p-aminobenzyloxycarbonyl group (PAB). The same mechanism for release of MMAE is operative for polatuzumab-vedotin (Polivy®). Other ADCs in pivotal trials that employ protease/peptidase-sensitive linkers are SYD985, ADCT-402, ASG-22CE and DS-8201a. Protease-mediated release of payload is also part of the design of RG7861 (DSTA4637S), which is an ADC under development in an area outside oncology, specifically for treatment of bacterial infections.

Two ADCs have been approved (Besponsa® and Mylotarg®) that consist of an antibody connected to a DNA-damaging payload (calicheamicin) via an acid-sensitive group, in particular a hydrazone group. Similarly, sacituzumab govetican, an ADC in phase III clinical studies, employs release of payload via acidic hydrolysis of a carbonate group. A glutathione-sensitive disulfide group is part of the linker in mirvetuximab soravtansine to connect antibody to the maytansinoid payload DM4 and also in IMGN853. Currently, more than 75 ADCs are in various stages of clinical trials, the at least 70% of which contain one form of a cleavable linker.

As described above, a self-immolative unit is part of the linker in many ADCs, which in most cases at least exists of an (acylated) para-aminobenzyl unit connected to a protease-sensitive peptide fragment for enzymatic release of the amino group. Besides the aminobenzyl group, other aromatic moieties may also be employed as part for the self-immolative unit, for example heteroaromatic moieties such as pyridine or thiazoles, see for example US7,754,681 and US2005/0256030. Substitution of the aminobenzyl group may be in the para position or in the ortho position, in both cases leading the same 1,6-elimination mechanism. The benzylic position may be substituted with alkyl or carbonyl derivatives, for example esters or amides derived from mandelic acid, as for example disclosed in WO2015/038426, incorporated by reference. The benzylic position of the self-immolative unit is connected to a heteroatom leaving group, typically based on, but not limited to, oxygen or nitrogen. Predominantly, the benzylic functional group exists of a carbamate moiety, which will release carbon dioxide upon triggering of the 1,6-elimination mechanism, and a primary or secondary amino group. The primary or secondary amino group may be part of the toxic payload itself, and may be an aromatic amino group or an aliphatic amino group. In the latter case, the amino group of the liberated payload will most likely have a pKa higher than and therefore be mostly in a protonated state at physiological conditions (pH 7-7.5), and specifically in the acidic environment of the tumour (pH <7).

The primary or secondary amino group may also be part of another self-immolative group, for example an N,N-dialkylethylenediamine moiety. The N,N-dialkylethylenediamine moiety at the other may be connected to another carbamate group to liberate, upon cyclization, an alcohol group as part of the toxic payload, as for example demonstrated by Elgersma et al, Mol. Pharm.2015, 12, 1813-1835, incorporated by reference. The primary or secondary amino group of the carbamate moiety may also form part of an N,O-acetal, a method which has been used in several drug delivery strategies, for example to release 5-fluorouracil (Madec-Lougerstay et al, J. Chem. Soc. Perkin Trans I, 1999, 1369-1375) and SN-38 (Santi et al, J. Med. Chem. 2014, 57, 2303-2314). Most recently, a similar configuration was employed by Kolakowski et al, Angew. Chem. Int. Ed. 2016, 55, 7948-7951, incorporated by reference, for design of linkers with prolonged serum exposure because of the long circulation time of ADCs, in combination with a beta-glucuronidase-promoted release mechanism, to release aliphatic alcohols. The functional group at the benzylic position of the self-immolative aromatic moiety may also be a phenolic oxygen, see for example Toki et al, J. Org. Chem. 2002, 67, 1866-1872 and US7,553,816, incorporated by reference, however not an aliphatic alcohol because an aliphatic alcohol does not possess sufficient leaving group capacity (typical pKa 13-15). Another option for the benzylic functional group is a quaternary ammonium group, which will release a trialkylamino group or a heteroaryl amine upon elimination, as reported by Burke et al, Mol. Cancer Ther. 2016, 15, 938-945 and Staben et al, Nat. Chem. 2016, 8, 1112-1119, incorporated by reference.

Currently, payloads utilized in ADCs primarily include microtubule-disrupting agents [e.g. monomethyl auristatin E (MMAE) and maytansinoid-derived DM1 and DM4], DNA-damaging agents [e.g., calicheamicin, pyrrolobenzodiazepines (PBD) dimers, indolinobenzodiapines dimers, duocarmycins, anthracyclins], topoisomerase inhibitors [e.g. SN-38, exatecan and derivatives thereof, simmitecan] or RNA polymerase II inhibitors [e.g. amanitin]. Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948 and for example studied by Li et al, Cancer Res. 2016, 76, 2710-2719. Generally spoken, cytotoxic payloads that are neutral will show bystander killing whereas ionic (charged) payloads do not, as a consequence of the fact that ionic species do not readily pass a cellular membrane by passive diffusion. For example, evaluation of a range of exatecan derivatives indicated that acylation of the primary amine with hydroxyacetic acid provided a derivative (DXd) with substantially enhanced bystander killer versus various aminoacylated exatecan derivatives, as disclosed by Ogitani et al, Cancer Sci. 2016, 107, 1039-1046, incorporated by reference.

A disadvantage of the majority of the clinically tested and marketed ADCs in the field is that the toxic payload may induce dose-limiting off-target toxicities, reviewed by Donaghy et al, MAbs 2016, 8, 659-71, incorporated by reference. It was for example demonstrated by Thon et al. Blood 2012, 120, 1975-84, incorporated by reference, that ADCs can be taken up by differentiating hematopoietic stem cells, leading to release of toxic payload, inhibition of megakaryocyte proliferation and differentiation, thus preventing the generation of thrombocytes and finally resulting in thrombocytopenia. Similarly, it is believed that hydrazone linker instability played a role in the safety issues of Mylotarg®, which was withdrawn from the market in 2010 (but later re-introduced). It has been shown that linkers designed for proteolytic cleavage by cathepsins can also be cleaved by other enzymes like esterase Ces1c (reported by Dorywalska et al, Mol. Cancer Ther. 2016, 15, 958-970, incorporated by reference). In fact, it was demonstrated by Caculitan et al, Cancer Res. 2017, 7027-7037, incorporated by reference, that even in the absence of cathepsin B, peptide-based cleavable linkers readily undergo cellular processing to release free payload. Moreover, it was demonstrated by Zhao et al. (Mol. Cancer Ther. 2017, 16, 1866-1876, incorporated by reference) that excretion of elastase by differentiating neutrophils may cause premature release of toxic payload, and is one of the causes of neutropenia, a common adverse event in cancer patients treated with MMAE-based ADCs.

Antibody conjugates known in the art may suffer from several disadvantages. For antibody-drug conjugates, a measure for the loading of the antibody with a toxin is given by the drug-antibody ratio (DAR), which gives the average number of active substance molecules per antibody. In general, two general approaches can be identified for the generation of an ADC, one via random (stochastic) conjugation to endogenous amino acids and one involving conjugation to one or more specific sites in the antibody, which may be a native site in the antibody or a site engineered into the antibody for such purpose.

Processes for the preparation of an ADC by stochastic conjugation generally result in a product with a DAR between 2.5 and 4, but in fact such an ADC comprises a mixture of antibody conjugates with a number of molecules of interest varying from 0 to 8 or higher. In other words, antibody conjugates by stochastic conjugation generally are formed with a DAR with high standard deviation. For example, gemtuzumab ozogamicin is a heterogeneous mixture of 50% conjugates (0 to 8 calicheamycin moieties per IgG molecules with an average of 2 or 3, randomly linked to solvent exposed lysine residues of the antibody) and 50% unconjugated antibody (Bross et al., Clin. Cancer Res. 2001, 7, 1490; Labrijn et al., Nat. Biotechnol. 2009, 27, 767, both incorporated by reference). For brentuximab vedotin (Adcetris®), Kadcyla® (T-DM1), and other ADCs in the clinic, it is still uncontrollable exactly how many drugs are attaching to any given antibody and therefore the ADC is obtained as a statistical distribution of conjugates with the majority having DAR3-4. One approach to achieve a higher DAR is by reduction of all (4) interchain disulfide bonds in a monoclonal antibody, thereby liberating a total of 8 cysteine side chains as free thiols, followed by global conjugation with maleimide-functionalized payload, to reach a final DAR between 6-8. This methodology is applied in various clinical stage ADCs, including for example IMMU-132, IMMU-110, DS-8201a, U3-1402, SGN-CD48a and SGN-CD228A and can be applied to a variety of payloads, however, is less suitable for antibodies other than IgG1 due to fragment scrambling during the reduction step.

Many technologies are known for bioconjugation, as summarized in G.T. Hermanson, “Bioconjugate Techniques”, Elsevier, 3^{rd} Ed. 2013, incorporated by reference. Two main technologies can be recognized for the preparation of ADCs by random conjugation, either based on acylation of lysine side-chain or based on alkylation of cysteine side-chain. Acylation of the ε-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in the manufacuting of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Another important technology for conjugation to cysteine side-chain is by means of disulfide bond, a bioactivatable connection that has been utilized for reversibly connecting protein toxins, chemotherapeutic drugs, and probes to carrier molecules (see for example Pillow et al., Chem. Sci. 2017, 8, 366-370. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, DOl: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/ (accessed Jan. 7^{th}, 2020). Reaction with methylsulfonylphenyloxadiazole has also been reported for cysteine conjugation by Toda et al., Angew. Chem. Int. Ed. 2013, 52, 12592-12596, incorporated by reference.

Although the majority (~65%) of clinical ADCs are based on random payload attachment, a clear trend toward site-specifically conjugated ADCs, based on the observation that site-specific ADCs come with an improved therapeutic index. To this end, a number of processes have been developed that enable the generation of an antibody-drug conjugate with defined DAR, by site-specific conjugation to a (or more) predetermined site(s) in the antibody. Site-specific conjugation is typically achieved by engineering of a specific amino acid (or sequence) into an antibody, serving as the anchor point for payload attachment, see for example Aggerwal and Bertozzi, Bioconj. Chem. 2014, 53, 176-192, incorporated by reference, most typically engineering of cysteine. Besides, a range of other site-specific conjugation technologies has been explored in the past decade, most prominently genetic encoding of a non-natural amino acid, e.g. p-acetophenylalanine suitable for oxime ligation, or p-azidomethylphenylalanine suitable for click chemistry conjugation. The majority of approaches based on genetic reengineering of an antibody lead to ADCs with a DAR of ~2. An alternative approach to antibody conjugation without reengineering of antibody involves the reduction of interchain disulfide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ~4 (DAR4).

It has been shown in WO2014065661, by van Geel et al., Bioconj. Chem. 2015, 26, 2233-2242 and Verkade et al., Antibodies 2018, 7, 12, all incorporated by reference, that homogeneous ADCs can be prepared and selectively tailored to DAR2 or DAR4 based on enzymatic remodeling of the native antibody glycan at N297 (trimming by endoglycosidase and introduction of azido-modified GalNAc derivative under the action of a glycosyltransferase) followed by attachment of cytotoxic payload using click chemistry. ADCs prepared by this technology were found to display a significantly expanded therapeutic index versus a range of other conjugation technologies and the technology of glycan-remodeling conjugation currently clinically applied in for example ADCT-601 (ADC Therapeutics).

A similar enzymatic approach to convert an antibody into an azido-modified antibody, reported by Lhospice et al., Mol. Pharmaceut. 2015, 12, 1863-1871, incorporated by reference, employs the bacterial enzyme transglutaminase (BTG or TGase). It was shown that deglycosylation of the native glycosylation site N297 with PNGase F liberates the neighbouring N295 to become a substrate for TGase-mediated introduction, which converts the deglycosylated antibody into a bisazido antibody upon subjection to an azide-bearing molecule in the presence of TGase. Subsequently, the bis-azido antibody was reacted with DBCO-modified cytotoxins to produce ADCs with DAR2. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

Other methods to introduce azides into antibodies have been reported based on prior genetic modification of antibody followed by introduction of non-natural amino acids using genetic encoding based on AMBER suppression codon, as for example demonstrated by Axup et al.. Proc. Nat. Acad. Sci. 2012, 109, 16101-16106, incorporated by reference. Similarly, Zimmerman et al., Bioconj. Chem. 2014, 25, 351-361, incorporated by reference have employed a cell-free protein synthesis method to introduce azidomethylphenylalanine (AzPhe) intro monoclonal antibodies for conversion into ADC by means of metal-free click chemistry. Also in this case, ADCs with DAR2 are prepared, or DAR4 in case two AzPhe amino acids are introduced first. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of (copper-catalyzed) click chemistry. Finally, genetic encoding of aliphatic azides in recombinant proteins using a pyrrolysyl-tRNA synthetase/tRNA_{CUA} pair was shown by Nguyen et al., J. Am. Chem. Soc. 2009, 131, 8720-8721, incorporated by reference and labelling was secured by click chemistry. The latter method should also be applicable to produce DAR2 ADCs, similar to the method reported by Oller-Salvia et al., Angew. Chem. Int. Ed. 2018, 57, 2831-2834.

It has also been shown by Bruins et al., Bioconjugate Chem. 2017, 28, 1189-1193, incorporated by references, that antibodies can be site-specifically conjugated to cytotoxic payload by tyrosinase-mediated oxidation of a suitably positioned tyrosine through an intermediate 1,2-quinone that subsequently can undergo cycloaddition with a strained alkyne or alkene.

Chemical approaches have also been developed for site-specific modification of antibodies without prior genetic modification, as for example highlighted by Yamada and Ito, ChemBioChem. 2019, 20, 2729-2737.

Chemical conjugation by affinity peptide (CCAP) for site-specific modification has been developed by Kishimoto et al., Bioconj. Chem. 2019, by using a peptide that binds with high affinity to human IgG-Fc, thereby enabling selective modification of a single lysine in the Fc-fragment with a biotin moiety or a cytotoxic payload. Similarly, Matsuda et al., ACS Omega 2019, 4, 20564-20570 have demonstrated that a similar approach (AJICAP™ technology) can be applied for the site-specific introduction of thiol groups on a single lysine in the antibody heavy chain. CCAP or AJICAP™ technology may also be employed for the introduction of azide groups or other functionalities.

While the mainstream of ADCs on the market and in the clinic have a drug loading somewhere between 2 and 8, as described above, for some highly cytotoxic payloads, such as most of the PBD dimers the related IGN-type payloads, but also enediyne-based payloads, amanitins and others, a lower DAR would be preferable. It has been found that the maximum tolerated dose in humans for extremely potent payloads may reduce to a value well below 1 mg/kg, often even below <300 µg/kg or even <100 µg/kg. As a consequence, in vivo receptor saturation is not reached after administration (typically intravenously), leading to suboptimal tumor uptake and enhanced clearance. For such cases, a DAR1 format with the same payload could be preferable, as the MTD versus the similar DAR2 version will likely be two times higher. Ruddle et al., ChemMedChem 2019, 14, 1185-1195 have recently shown that DAR1 conjugates can be prepared from antibody Fab fragments (prepared by papain digestion of full antibody or recombinant expression) by selective reduction of the C_{H}1 and C_{L} interchain disulfide chain, followed by rebridging the fragment by treatment with a symmetrical PDB dimer containing two maleimide units. The resulting DAR1-type Fab fragments were shown to be highly homogeneous, stable in serum and show excellent cytotoxicity. In a follow-up publication, White et al., MAbs 2019, 11, 500-515, and also in WO2019034764, incorporated by reference, it was shown that DAR1 conjugates can also be prepared from full IgG antibodies, after prior engineering of the antibody: either an antibody is used which has only one intrachain disulfide bridge in the hinge region (Flexmab technology, reported in Dimasi et al., J. Mol. Biol. 2009, 393, 672-692, incorporated by reference) or an antibody is used which has an additional free cysteine, which may be obtained by mutation of a natural amino acid (e.g. HC-S239C) or by insertion into the sequence (e.g. HC-i239C, reported by Dimasi et al., Mol. Pharmaceut. 2017, 14, 1501-1516). Either engineered antibody was shown to enable the generation of DAR1 ADCs by reaction of the resulting cysteine-engineered ADC with a bis-maleimide derived PBD dimer. It was shown that the Flexmab-derived DAR1 ADCs was highly resistant to payload loss in serum and exhibited potent antitumor activity in a HER2-positive gastric carcinoma xenograft model. Moreover, this ADC was tolerated in rats at twice the dose compared to a site-specific DAR2 ADC prepared using a single maleimide-containing PBD dimer. However, no improvement in therapeutic window was noted, since the minimal effective dose (MED) of the DAR1 ADC versus the DAR2 ADC increased with the same factor 2.

To date, no DAR1 technology has been reported that improves the therapeutic index versus DAR2 ADCs. Also, no technology has been reported for the generation by DAR1 ADCs from full antibodies without requiring reengineering of the monoclonal antibody. Both improvement of therapeutic index and/or a non-genetic approach towards DAR1 ADCs would represent significant contribution for the development of better ADCs with faster time-to-clinic.

### SUMMARY OF THE INVENTION

A technology is presented to convert any full-length antibody into a stable and site-specific ADC with a single drug load (DAR1), without requiring prior reengineering of the antibody. The technology is applicable to any IgG isotype and enables the attachment of payloads, ranging from small molecule cytotoxics to protein scaffolds (cytokines, scFvs) to oligonucleotides and others, to antibodies via a cycloaddition conjugation reaction. The procedure according to a preferred embodiment, which involves prior trimming of a glycan with endoglycosidase proceeds with concomitant abrogation of Fc-gamma receptor binding, thus removing effector function.

The antibody-payload conjugate according to the invention is according to structure (1): 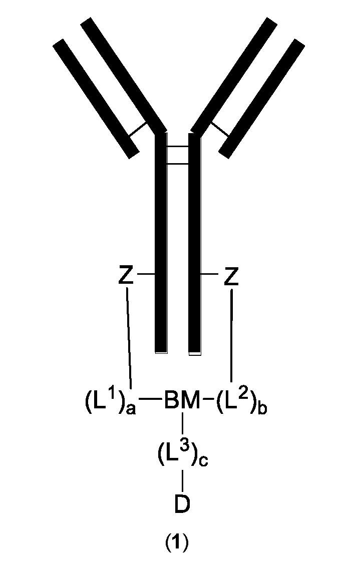 wherein:
a, b and c are each independently 0 or 1;
L¹, L² and L³ are linkers;
D is a payload;
BM is a branching moiety;
Z are connecting groups obtainable by a cycloaddition reaction.

The invention further provides a method for preparing the antibody-payload conjugate according to the invention, an intermediate compound in that preparation method, and medical uses of the antibody-payload conjugate according to the invention.

### DESCRIPTION OF THE DRAWINGS

FIG. 1 shows a representative (but not comprehensive) set of functional groups (F) in a biomolecule, either naturally present or introduced by engineering, which upon reaction with a reactive group lead to connecting group Z. Functional group F may be artificially introduced (engineered) into a biomolecule at any position of choice. The pyridazine connecting group (bottom line) is the product of the rearrangement of the tetrazabicyclo[2.2.2]octane connecting group, formed upon reaction of tetrazine with alkyne, with loss of N₂. Herein, X may be halogen and X⁹ may be H, alkyl or pyridyl. Connecting groups Z of structure (10e) - (10h) are preferred connecting groups to be used in the present invention.

FIG. 2 shows several structures of derivatives of UDP sugars of galactosamine, which may be modified with e.g. an azidoacetyl group (11b), or an azidodifluoroacetyl group (11c) at the 2-position, or with an azido group at the 6-position of N-acetyl galactosamine (11d). The monosaccharide (i.e. with UDP removed) are preferred moieties Su to be used in the present invention.

FIG. 3 shows the general process for non-genetic conversion of a monoclonal antibody into a glycan-remodeled antibody, which contains two azido groups (one on either native glycosylation site). Upon reaction with a bivalent cyclooctyne construct, a single payload (R) is attached to the bis-azido antibody. Such clipping can also be achieved by copper-catalyzed click reaction using a bivalent construct with two terminal acetylene groups (not depicted).

FIG. 4 shows cyclooctynes suitable for metal-free click chemistry. The list is not comprehensive, for example alkynes can be further activated by fluorination, by substitution of the aromatic rings or by introduction of heteroatoms in the aromatic ring.

FIG. 5 shows examples of the R group that is present in the bivalent constructs of FIGS. 3 and 4, which is defined as the payload in the antibody-drug conjugate. The R-group may attached to the bivalent construct via a cleavable moiety, for example a peptide-cleavable linker as depicted in the top structure. Acid-cleavable or disulfide-based linkers may also be used (not depicted), or linker that are cleaved by yet another mechanism. The R-group may also be attached via a non-cleavable linker (bottom structure). The R-group itself may for example be a cytotoxic molecule (but is not limited to cytotoxic molecules).

FIG. 6 is an illustration of a bivalent cyclooctyne construct suitable for generation of DAR1 ADCs by clipping onto bis-azido antibody, wherein the two cyclooctyne moieties are attached to two sites of a payload with a dimeric structure, for example a PBD dimer or duocarmycin dimer. The linker may be of cleavable nature or non-cleavable nature, as illustrated for the PBD dimer. The dimeric cytotoxic payload is not necessarily symmetrical in nature as for the examples illustrated, for example a combination of a duocarmycin monomer and a PBD monomer is also possible.

FIG. 7 illustrates an indirect approach for attachment of payload in a DAR1 format by using a trivalent cyclooctyne construct that reacts with the bisazido-mAb leaving one cyclooctyne free for subsequent click chemistry (illustrated with azide-modified payload, other options may be click chemistry with nitrones, nitrile oxides, diazo compounds, tetrazines, etcetera).

FIG. 8 shows various options for trivalent constructs for reaction with a bis-glycan modified mAb. The trivalent construct may be homotrivalent or heterotrivalent (2+1 format). A homotrivalent contract (X = Y) may consist of 3× cyclooctyne or 3× acetylene. A heterotrivalent construct (X ≠ Y) may for example consist of two cyclooctyne groups and one maleimide group or one trans-cyclooctene group. The heterotrivalent construct may exist of any combination of X and Y unless X and Y and reactive with each other (e.g. BCN + tetrazine).

FIG. 9 shows a range of bivalent BCN reagents (105, 107, 118, 125, 129, 134), trivalent BCN reagents (143, 145, 150), monovalent BCN reagents for sortagging (157, 161, 163, 168) or monovalent tetrazine reagent for sortagging (154).

FIG. 10 shows a range of bivalent or trivalent cross-linkers (XL07-XL13).

FIG. 11 shows a range of antibody variants as starting materials for subsequent conversion to antibody conjugates

FIG. 12 shows a range of bis-BCN-modified cytotoxic drugs based on MMAE or MMAF for generation of DAR1 ADCs by cross-linking with bis-azido-modified antibody.

FIG. 13 shows a range of additional bis-BCN-modified cytotoxic drugs based on MMAE (303), PBD dimer (304), calicheamicin (305) or PNU159,682 (306) for generation of DAR1 ADCs by cross-linking with bis-azido-modified antibody.

FIG. 14 shows a range of bivalent cytotoxic drugs with various cyclooctynes (BCN, DIBO, DBCO, with various inter-cyclooctyne linker variations) or azide, based on MMAE or MMAF for generation of DAR1 ADCs by cross-linking with bis-azido-modified antibody or bis-alkyne-modified antibody.

FIG. 15 shows the structure of two monovalent, linear linker-drugs based on BCN-MMAE (312) or azide-MMAF (313).

FIG. 16 shows SDS-PAGE analysis: Lane 1 - rituximab; Lane 2 - rit-v1a; Lane 3 - rit-v1a-145; Lane 4 - rit-v1a-(201)₂; Lane 5 - rit-v1a-145-204; Lane 6 - rit-v1a-145-PF01; Lane 7 -rit-v1a-145-PF02. Gels were stained with coomassie to visualize total protein. Samples were analyzed on a 6% SDS-PAGE under non-reducing conditions (left) and 12% SDS-PAGE under reducing conditions (right).

FIG. 17 shows RP-HPLC traces of B12-v1a (upper trace) and B12-v1a-145 (lower trace). Samples have been digested with IdeS prior to RP-HPLC analysis.

FIG. 18 shows SDS-PAGE analysis: Lane 1 -trast-v1a; Lane 2 -trast-v1a-XL11; Lane 3 and 4 - trast-v1a-XL11-PF01; Lane 5 - rit-v1a; Lane 6 - rit-v1a-XL11; Lane 7 and 8 - rit-v1a-XL11-PF01. Gels were stained with coomassie to visualize total protein. Samples were analyzed on a 6% SDS-PAGE under non-reducing conditions (left) and 12% SDS-PAGE under reducing conditions (right).

FIG. 19 shows the RP-HPLC data for deglycosylated trastuzumab after treatment with bis-BCN-MMAE LD03 (=303)

FIG. 20 shows SDS-PAGE analysis on a 6% gel under non-reducing conditions: Lane 1 rituximab; Lane 2rit-v1a-(201)₂; Lane 3 - rit-v1a-145-PF08; Lane 4 - B12-v1a-145-PF01; Lane 5 B12-v1a-145-PF08. Gels were stained with coomassie to visualize total protein. Lanes 1 and 2 are included as a reference for non-conjugated mAb and 2:2 molecular format.

FIG. 21 shows SDS-PAGE analysis on a 6% gel under non-reducing conditions: Lane 1 rit-v1a-(201)₂; Lane 2 - rit-v1a-145-PF01; Lane 3 - rit-v1a; Lane 4 - rit-v1a-PF22; Lane 5 -trast-v1a-PF22. Gels were stained with coomassie to visualize total protein. Lanes 1 and 2 are included as a reference for non-conjugated mAb and 2:2 molecular format.

FIG. 22 shows SDS-PAGE analysis on a 6% gel under non-reducing conditions: Lane 1 trast-v1a; Lane 2trast-v1a-PF23. Gels were stained with coomassie to visualize total protein. Lanes 1 is included as a reference for non-conjugated mAb.

FIG. 23 shows SDS-PAGE analysis on a 6% gel under non-reducing conditions: Lane 1 rit-v1a; Lane 2rit-v1a-(201)₂; Lane 3 - rit-v1a-145-PF01; Lane 4 - rit-v1a-PF22; Lane 5 - rit-v1a-PF23. Gels were stained with coomassie to visualize total protein. Lanes 1-4 are included as a reference for non-conjugated mAb, 2:1 and 2:2 molecular format.

FIG. 24 shows SDS-PAGE analysis on a 6% gel under non-reducing conditions: Lane 1 rit-v1a-145; Lane 2rit-v1a-145-PF09; Lane 3 - trast-v1a-145; Lane 4 - trast-v1a-145-PF09; Lane 5 - rit-v1a; Lane 6 - rit-v1a-(PF07)₂; Lane 7 - trast-v1a; Lane 8 - trast-v1a-(PF07)₂. Gels were stained with coomassie to visualize total protein.

FIG. 25 shows non-reducing SDS-page analysis: lane 1 - Trast-v1a-(PF.)₁₋₂; lane 2 -trast-v1a-(209)₁₋₂; lane 3 - trast-v1a-(PF11)₁₋₂; lane 4 - trast-v1a; lane 5 - trast-v1a-145-PF12; lane 6 - trast-v1a-145. Gels were stained with coomassie to visualize total protein.

FIG. 26 shows SDS-PAGE analysis on a 6% gel under non-reducing conditions: Lane 1 rit-v1a-145; Lane 2rit-v1a-145-PF17; Lane 3 - trast-v1a-145; Lane 4 - trast-v1a-145-PF17. Gels were stained with coomassie to visualize total protein.

FIG. 27 shows SDS-PAGE analysis on a 6% gel under non-reducing conditions: Lane 1 trast-v1a; Lane 2trast-v1a-PF29; Lane 3 - rit-v1a; Lane 4 - rit-v1a-PF29. Gels were stained with coomassie to visualize total protein.

FIG. 28 shows effect of bispecifics based on hOKT3 200 on RajiB Tumor cell killing with human PBMCs. Bispecifics and calculated EC₅₀ values are shown in the legend. B12-v1a-145-PF01 was included as a negative control.

FIG. 29 shows effect of bispecifics based on anti-4-1BB PF31 on RajiB Tumor cell killing with human PBMCs. Bispecifics and calculated EC₅₀ values are shown in the legend. B12-v1a-145-PF31 was included as a negative control.

FIG. 30 shows cytokine levels in supernatants of a RajiB-PBMC co-culture after incubation with bispecifics based on hOKT3 200. The murine OKT3 mlgG2a antibody (Invitrogen 16-0037-81) was included as a positive control.

FIG. 31 shows cytokine levels in supernatants of a RajiB-PBMC co-culture after incubation with bispecifics based on anti-4-1BB PF31. The murine OKT3 mlgG2a antibody (Invitrogen 16-0037-81) was included as a positive control.

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application. When the structure of a compound is depicted as a specific cis or trans isomer, it is to be understood that the invention of the present application is not limited to that specific cis or trans isomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “monosaccharide” is herein used in its normal scientific meaning and refers to an oxygen-containing heterocycle resulting from intramolecular hemiacetal formation upon cyclisation of a chain of 5-9 (hydroxylated) carbon atoms, most commonly containing five carbon atoms (pentoses), six carbon atoms (hexose) or nine carbon atoms (sialic acid). Typical monosaccharides are ribose (Rib), xylose (Xyl), arabinose (Ara), glucose (Glu), galactose (Gal), mannose (Man), glucuronic acid (GlcA), N-acetylglucosamine (GlcNAc), N-acetylgalactosamine (GalNAc) and N-acetylneuraminic acid (NeuAc).

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art. Typical examples of antibodies include, amongst others, abciximab, rituximab, basiliximab, palivizumab, infliximab, trastuzumab, efalizumab, alemtuzumab, adalimumab, tositumomab-l131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “polar linker” is herein defined as a linker that contains structural elements with the specific aim to increase polarity of the linker, thereby improving aqueous solubility. A polar linker may for example comprise one or more units, or combinations thereof, selected from ethylene glycol, a carboxylic acid moiety, a sulfonate moiety, a sulfone moiety, an acylated sulfamide moiety, a phosphate moiety, a phosphinate moiety, an amino group or an ammonium group.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

An “activatable group” is herein defined as a functional group attached to an aromatic group that can undergo a biochemical processing step such as proteolytic hydrolysis of an amide bond or reduction of a disulphide bond, upon which biochemical processing step a self-immolative process of the aromatic group will be initiated. The activatable group may also be referred to as “activating group”.

A “bioconjugate” is herein defined as a compound wherein a biomolecule is covalently connected to a payload via a linker. A bioconjugate comprises one or more biomolecules and/or one or more payloads. Antibody-conjugates, such as antibody-payload conjugates and antibody- drug-conjugates are bioconjugates wherein the biomolecule is an antibody.

A “biomolecule” is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “payload” refers to the moiety that is covalently attached to a targeting moiety such as an antibody, but also to the molecule that is released from the conjugate upon cleavage of the linker. Payload thus refers to the monovalent moiety having one open end which is covalently attached to the targeting moiety via a linker, which is in the context of the present invention referred to as D, and also to the molecule that is released therefrom.

The terms “2:1 molecular format” refer to a protein conjugate consisting of a bivalent monoclonal antibody (IgG-type) conjugated to a single functional payload.

### Antibody-Payload Conjugate According to the Invention

The present invention relates to an antibody-payload conjugate having structure (1): 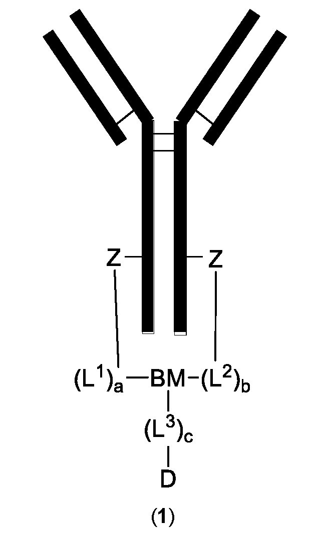 wherein:
a, b and c are each independently 0 or 1;
L¹, L² and L³ are linkers;
D is a payload;
BM is a branching moiety;
Z are connecting groups obtainable by a cycloaddition reaction.

In antibody-payload conjugate (1), payload D is connected to antibody AB, via connecting groups Z, optional linkers L¹, L² and L³ and branching moiety BM. In (1), a, b and c are each independently selected from 0 and 1. Preferred antibody-payload conjugates according to the invention have a = b = 1, i.e. both L¹ and L² are present, more preferably L¹ and L² are same. Especially preferred are symmetrical antibody-payload conjugates, wherein each occurrence of Z, a/b and L¹/L² is the same.

In a preferred embodiment, the antibody is conjugated via the glycan to payload D, in which case the antibody-payload conjugate according to the invention has structure (5): 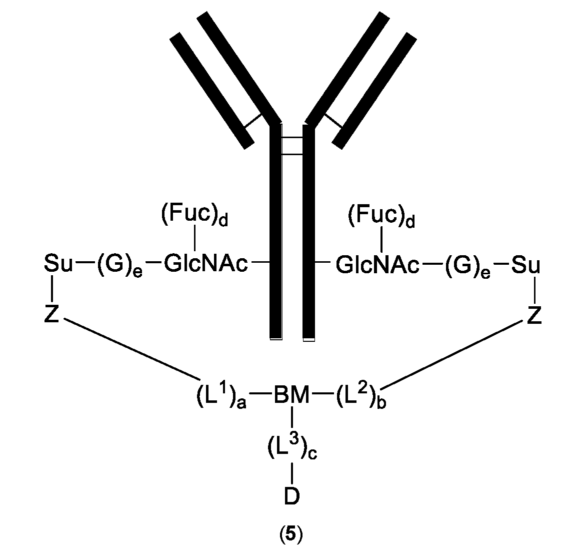 wherein:
e is an integer in the range of 0 10;
Su is a monosaccharide;
G is a monosaccharide moiety;
GlcNAc is an N-acetylglucosamine moiety;
Fuc is a fucose moiety
d is 0 or 1.

### Antibody AB

In (1), AB is an antibody. Preferably AB is a monoclonal antibody, more preferably selected from the group consisting of IgA, IgD, IgE, IgG and IgM antibodies. Even more preferably AB is an IgG antibody. The IgG antibody may be of any IgG isotype. The antibody may be any IgG isotype, e.g. IgG1, IgG2, Igl3 or IgG4. Preferably AB is a full-length antibody, but AB may also be a Fc fragment.

GlcNAc moieties in (5) are preferably present at a native N-glycosylation site in the Fc-fragment of antibody AB. Preferably said GlcNAc moieties are attached to an asparagine amino acid in the region 290-305 of AB. In a further preferred embodiment, the antibody is an IgG type antibody, and, depending on the particular IgG type antibody, said GlcNAc moieties are present on amino acid asparagine 297 (Asn297 or N297) of AB.

### Connecting Group Z

In antibody-payload conjugate (1) Z is a connecting group. As described in more detail above, the term “connecting group” refers to a structural element connecting one part of a compound and another part of the same compound. In (1), Z connects the antibody, possibly via a spacer, with branching moiety BM, via L¹ and/or L² if present. Whether L¹ and/or L² are present or not depends on the value of a and b. In a preferred embodiment, both occurrences of Z are the same.

As will be understood by the person skilled in the art, the nature of a connecting group depends on the type of cycloaddition reaction with which the connection between the parts of said compound was obtained. For example, Z may be obtainable by a [4+2] cycloaddition or a 1,3-dipolar cycloaddition.

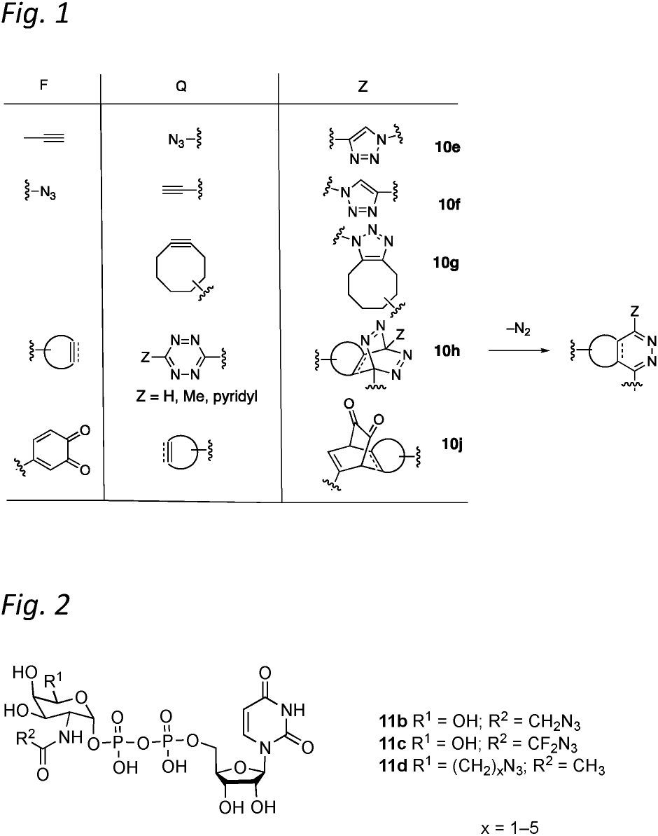

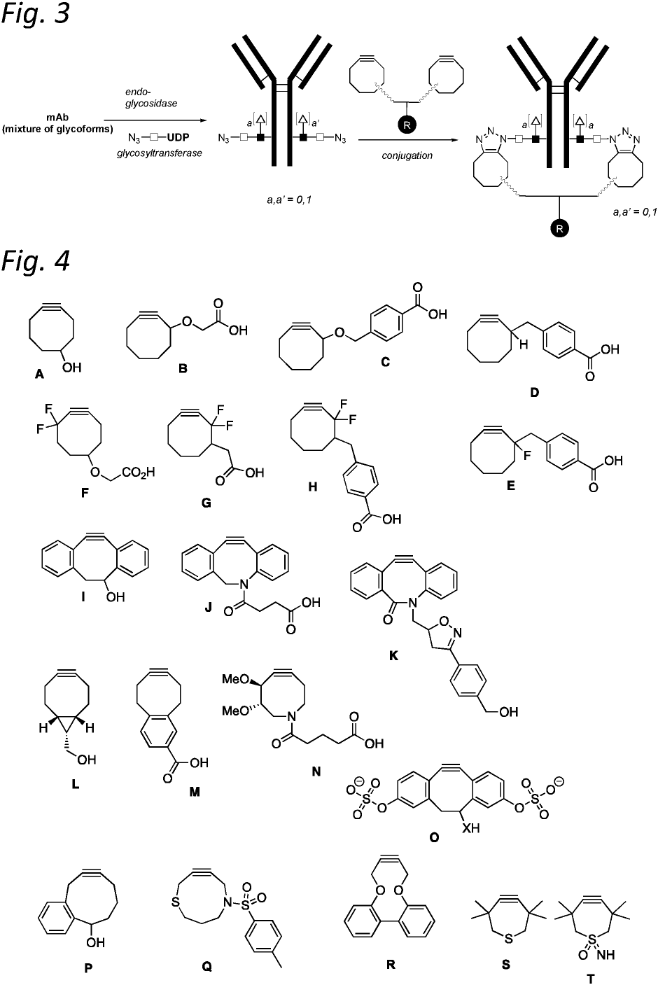

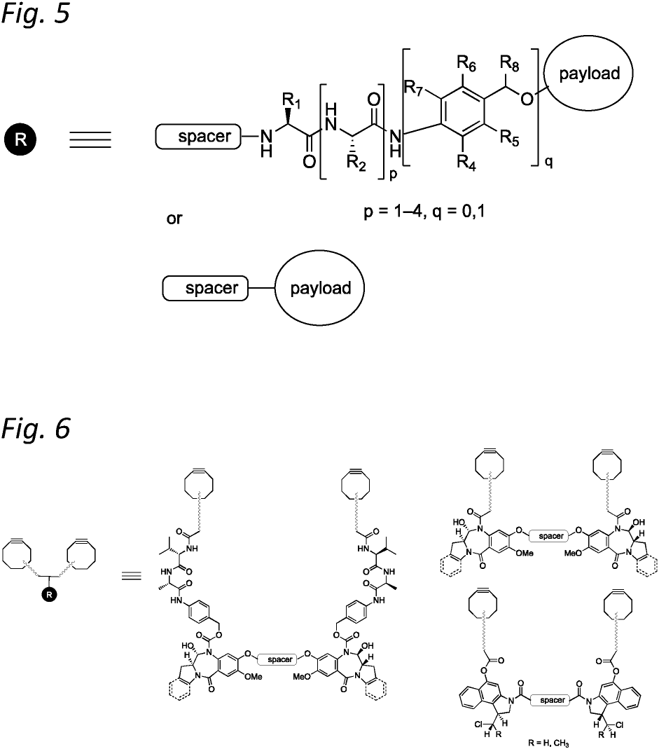

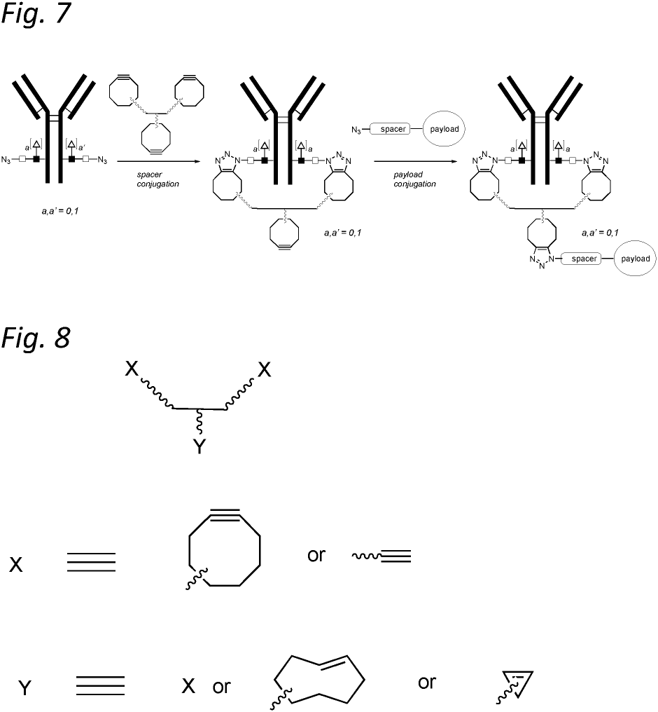

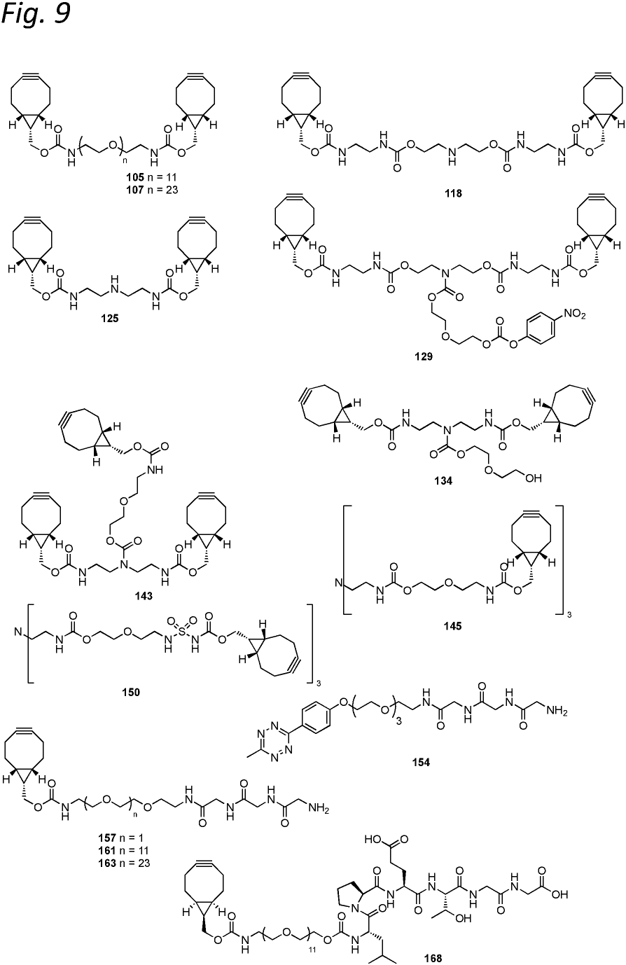

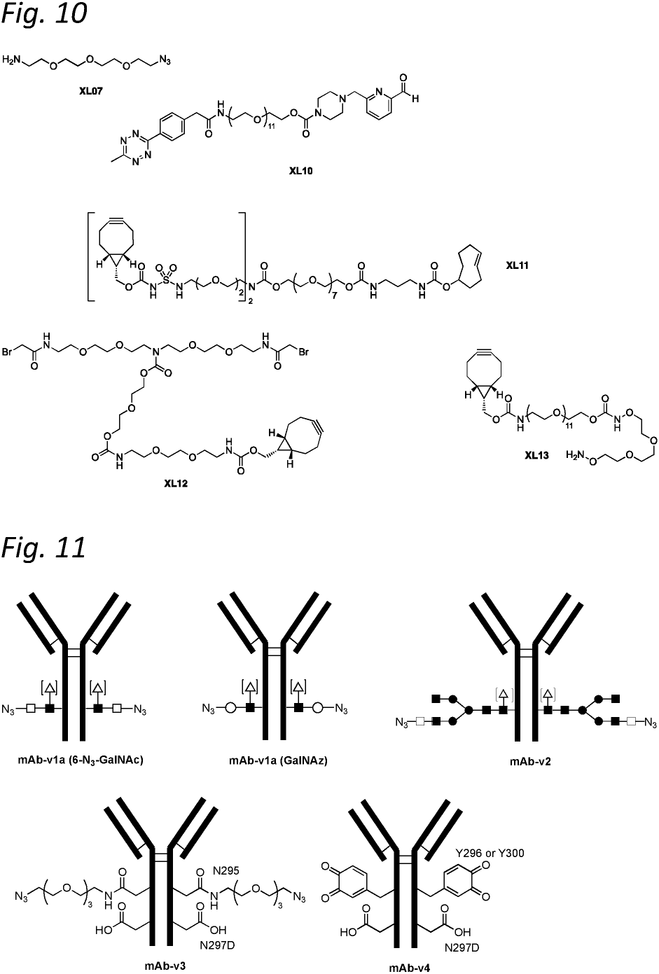

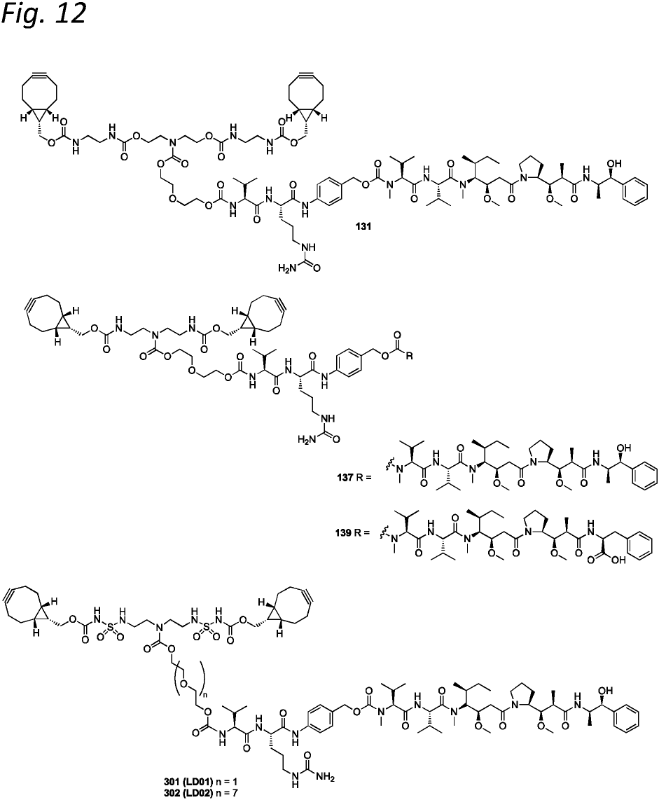

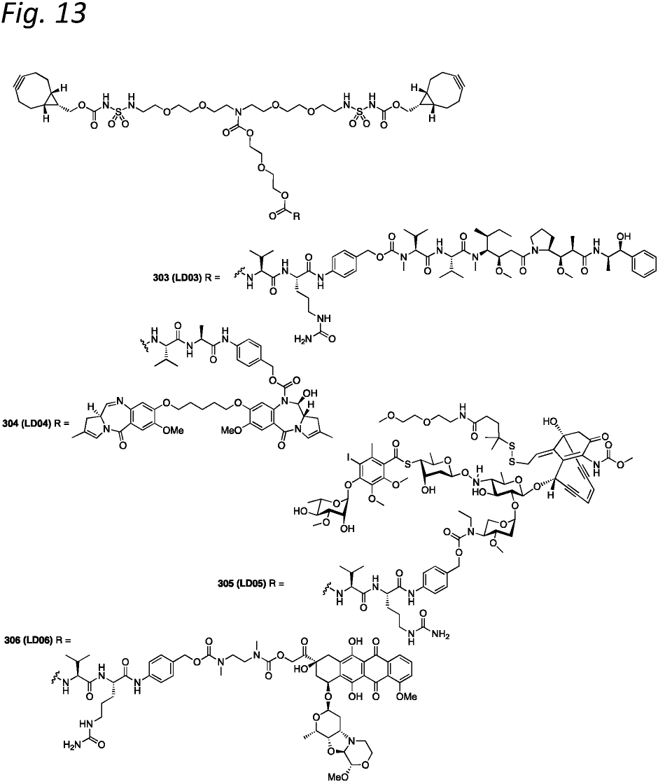

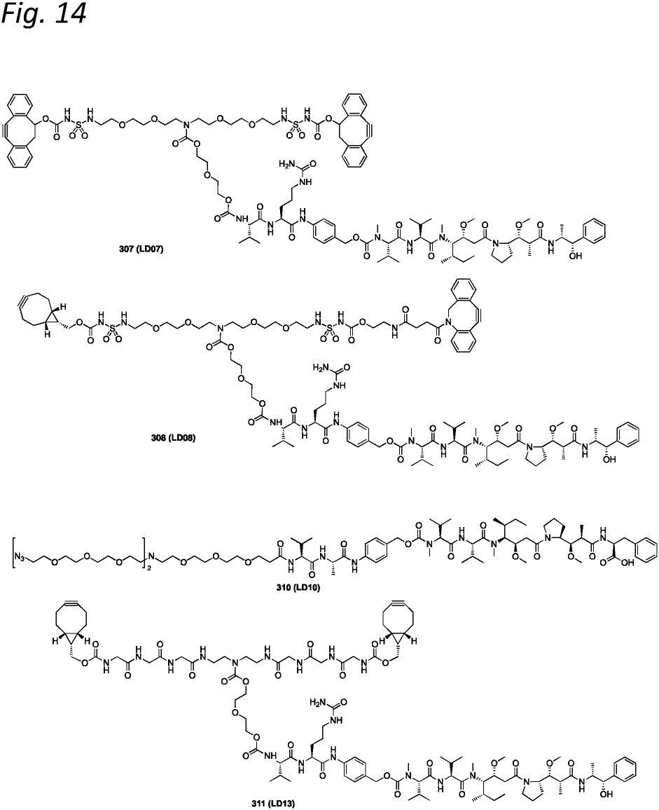

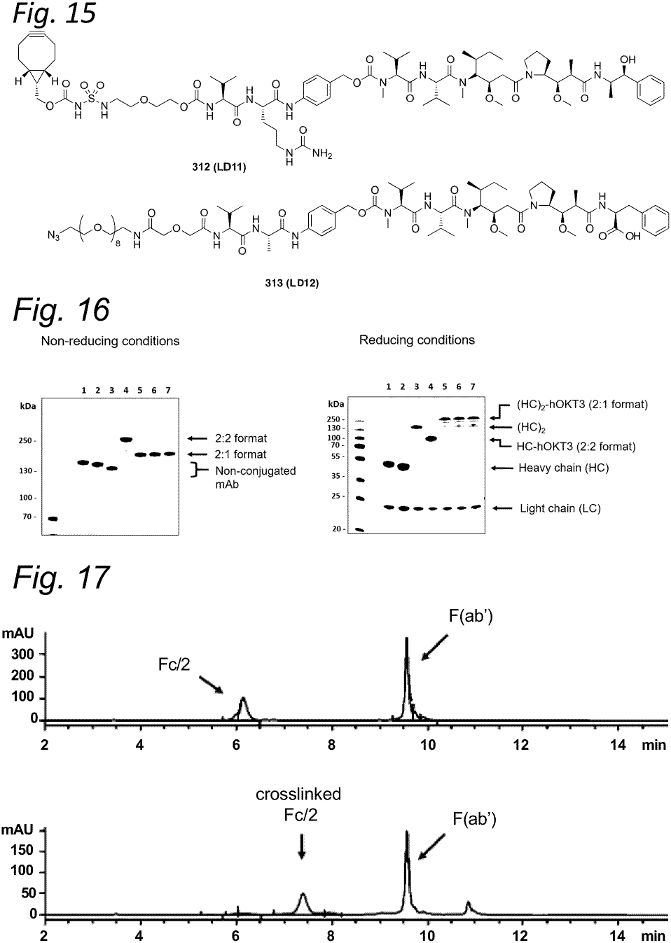

Cycloaddition reactions for the attachment of a reactive group Q to a reactive group F are known in the art. Consequently, a variety of connecting groups Z may be present in the conjugate according to the invention. In one embodiment, the connecting group Z is selected from the options described above, preferably as depicted in FIG. 1.

For example, when F comprises or is an alkynyl group, complementary groups Q include azido groups, and the corresponding connecting group Z is as shown in FIG. 1.

For example, when F comprises or is an azido group, complementary groups Q include alkynyl groups, and the corresponding connecting group Z is as shown in FIG. 1.

For example, when F comprises or is a cyclopropenyl group, a trans-cyclooctene group or a cycloalkyne group, complementary groups Q include tetrazinyl groups, and the corresponding connecting group Z is as shown in FIG. 1. In particular cases, Z is only an intermediate structure and will expel N₂, thereby generating a dihydropyridazine (from the reaction with alkene) or pyridazine (from the reaction with alkyne).

For example, when F comprises or is a tetrazinyl group, complementary groups Q include a cyclopropenyl group, a trans-cyclooctene group or a cycloalkyne group, and the corresponding connecting group Z is as shown in FIG. 1. In particular cases, Z is only an intermediate structure and will expel N₂, thereby generating a dihydropyridazine (from the reaction with alkene) or pyridazine (from the reaction with alkyne).

Additional suitable combinations of F and Q, and the nature of resulting connecting group Z are known to a person skilled in the art, and are e.g. described in G.T. Hermanson, “Bioconjugate Techniques”, Elsevier, 3rd Ed. 2013 (lSBN:978-0-12-382239-0), in particular in Chapter 3, pages 229 - 258, incorporated by reference. A list of complementary reactive groups suitable for bioconjugation processes is disclosed in Table 3.1, pages 230 - 232 of Chapter 3 of G.T. Hermanson, “Bioconjugate Techniques”, Elsevier, 3rd Ed. 2013 (ISBN:978-0-12-382239-0), and the content of this Table is expressly incorporated by reference herein.

In a preferred embodiment, connecting group Z is according to any one of structures (Za), (Ze) to (Zh), (Zj) and (Zk), as defined below. Preferably, Z is according to structures (Za), (Ze) or (Zj):

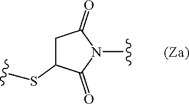

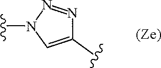

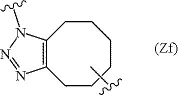

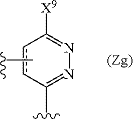

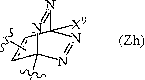

Herein,
X⁹ is selected from H, C₁₋₁₂ alkyl and pyridyl, wherein the C₁₋₁₂ alkyl preferably is C₁₋₄ alkyl, most preferably methyl.
In structure (Zg) and (Zh), the

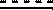

bond represents either a single or a double bond, and may be connected via either side of this bond to linkers L.
The wavy lines indicate the connection to linkers L. The connectivity depends on the specific nature of Q and F. Although either site of the connecting groups according to (Za) to (Zh) may be connected to L, it is preferred that the left-most of these groups as depicted is connected to (L¹)ₐ/(L²)_{b}.

Connecting group (Zh) typically rearranges to (Zg) with the liberation of N₂.

In a preferred embodiment, each Z independently contains a moiety selected from the group consisting of a triazole, a cyclohexene, a cyclohexadiene, an isoxazoline, an isoxazolidine, a pyrazoline, a piperazine, a thioether, an amide or an imide group. Triazole moieties are especially preferred to be present in Z.

In an especially preferred embodiment, connecting group Z comprises a triazole moiety and is according to structure (Zj):

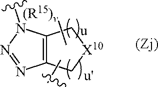

Herein, R¹⁵, X¹⁰, u, u′ and v are as defined for (Q36), and all preferred embodiments thereof equally apply to (Zj). The wavy lines indicate the connection to adjacent moieties (Su and (L¹)ₐ or (L²)_{b}), and the connectivity depends on the specific nature of Q and F. Although either site of the connecting group according to (Zj) may be connected to (L¹)ₐ/(L²)_{b}, it is preferred that the upper wavy bond as depicted represents the connectivity to Su. The connecting groups according to structure (Zf) and (Zk) are preferred embodiments of the connecting group according to (Zj).

In an especially preferred embodiment, connecting group Z comprises a triazole moiety and is according to structure (Zk):

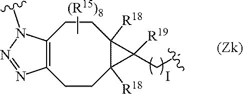

Herein, R¹⁵, R¹⁸, R¹⁹, and I are as defined for (Q37), and all preferred embodiments thereof equally apply to (Zj). The wavy lines indicate the connection to adjacent moieties (Su and (L¹)ₐ or (L²)_{b}), and the connectivity depends on the specific nature of Q and F. Although either site of the connecting group according to (Zj) may be connected to (L¹)ₐ, it is preferred that the left wavy bond as depicted represents the connectivity to Su

In a preferred embodiment, Q comprises or is an alkyne moiety and F is an azido moiety, such that connecting group Z comprises an triazole moiety. Preferred connecting groups comprising a triazole moiety are the connecting groups according to structure (Ze) or (Zj), wherein the connecting groups according to structure (Zj) is preferably according to structure (Zk) or (Zf). In a preferred embodiment, the connecting groups is according to structure (Zj), more preferably according to structure (Zk) or (Zf).

### The Branching Moiety BM

A “branching moiety” in the context of the present invention refers to a moiety that is embedded in a linker connecting three moieties. In other words, the branching moiety comprises at least three bonds to other moieties, one bond to reactive group F, connecting group Z or payload D, one bond to reactive group Q or connecting group Z, and one bond to reactive group Q or connecting group Z.

Any moiety that contains at least three bonds to other moieties is suitable as branching moiety in the context of the present invention. Suitable branching moieties include a carbon atom (BM-1), a nitrogen atom (BM-3), a phosphorus atom (phosphine (BM-5) and phosphine oxide (BM-6)), aromatic rings such as a phenyl ring (e.g. BM-7) or a pyridyl ring (e.g. BM-9), a (hetero)cycle (e.g. BM-11 and BM-12) and polycyclic moieties (e.g. BM-13, BM-14 and BM-15). Preferred branching moieties are selected from carbon atoms and phenyl rings, most preferably BM is a carbon atom. Structures (BM-1) to (BM-15) are depicted here below, wherein the three branches, i.e. bonds to other moieties as defined above, are indicated by * (a bond labelled with *).

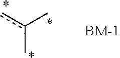

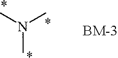

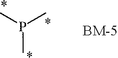

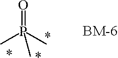

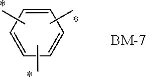

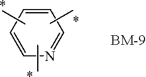

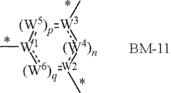

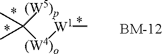

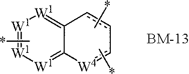

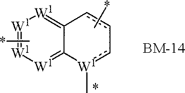

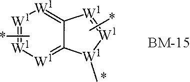

In (BM-1), one of the branches labelled with * may be a single or a double bond, indicated with

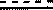

In (BM-11) to (BM-15), the following applies:
each of n, p, q and q is individually an integer in the range of 0 5, preferably 0 or 1, most preferably 1;
each of W¹, W² and W³ is independently selected from C(R²¹)_{w} and N;
each of W⁴, W⁵ and W⁶ is independently selected from C(R²¹)_{w+1}, N(R²²)_{w}, O and S;
each

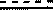

represents a single or double bond;
w is 0 or 1 or 2, preferably 0 or 1;
each R²¹ is independently selected from the group consisting of hydrogen, OH, C₁ - C₂₄ alkyl groups, C₁-C₂₄ alkoxy groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups, wherein the C₁ - C₂₄ alkyl groups, C₁ - C₂₄ alkoxy groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups are optionally substituted and optionally interrupted by one or more heteroatoms selected from O, S and NR³ wherein R³ is independently selected from the group consisting of hydrogen and C₁ - C₄ alkyl groups; and
each R²² is independently selected from the group consisting of hydrogen, C₁ - C₂₄ alkyl groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups, wherein the C₁ - C₂₄ alkyl groups, C₁ - C₂₄ alkoxy groups, C₃ - C₂₄ cycloalkyl groups, C₂ -C₂₄ (hetero)aryl groups, C₃ - C₂₄ alkyl(hetero)aryl groups and C₃ - C₂₄ (hetero)arylalkyl groups are optionally substituted and optionally interrupted by one or more heteroatoms selected from O, S and NR³ wherein R³ is independently selected from the group consisting of hydrogen and C₁ - C₄ alkyl groups.

The skilled person appreciates that the values of w and the bond order of the bonds represented by

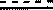

are interdependent. Thus, whenever an occurrence of W is bonded to an endocyclic double bond, w = 1 for that occurrence of W, while whenever an occurrence of W is bonded to two endocyclic single bonds, w = 0 for that occurrence of W. For BM-12, at least one of o and p is not 0.

Representative examples of branching moieties according to structure (BM-11) and (BM-12) include cyclopropyl, cyclobutyl, cyclopentyl, cyclohexyl, cycloheptyl, cyclooctyl, cyclopropenyl, cyclobutenyl, cyclopentenyl, cyclohexenyl, cycloheptenyl, cyclooctenyl, aziridine, azetidine, diazetidine, oxetane, thietane, pyrrolidine, dihydropyrrolyl, tetrahydrofuranyl, dihydrofuranyl, thiolanyl, imidazolinyl, pyrazolidinyl, oxazolidinyl, isoxazolidinyl, thiazolidinyl, isothiazolidinyl, dioxolanyl, dithiolanyl, piperidinyl, oxanyl, thianyl, piperazinyl, morpholino, thiomorpholino, dioxanyl, trioxanyl, dithyanyl, trithianyl, azepanyl, oxepanyl and thiepanyl. Preferred cyclic moieties for use as branching moiety include cyclopropenyl, cyclohexyl, oxanyl (tetrahydropyran) and dioxanyl. The substitution pattern of the three branches determines whether the branching moiety is of structure (BM-11) or of structure (BM-12).

Representative examples of branching moieties according to structure (BM-13) to (BM-15) include decalin, tetralin, dialin, naphthalene, indene, indane, isoindene, indole, isoindole, indoline, isoindoline, and the like.

In a preferred embodiment, BM is a carbon atom. In case the carbon atom is according to structure (BM-1) and has all four bonds to distinct moieties, the carbon atom is chiral. The stereochemistry of the carbon atom is not crucial for the present invention, and may be S or R. The same holds for the phosphine (BM-6). Most preferably, the carbon atom is according to structure (BM-1). One of the branches indicated with * in the carbon atom according to structure (BM-1) may be a double bond, in which case the carbon atom may be part of an alkene or imine. In case BM is a carbon atom, the carbon atom may be part of a larger functional group, such as an acetal, a ketal, a hemiketal, an orthoester, an orthocarbonate ester, an amino acid and the like. This also holds in case BM is a nitrogen or phosphorus atom, in which case it may be part of an amide, an imide, an imine, a phosphine oxide (as in BM-6) or a phosphotriester.

In a preferred embodiment, BM is a phenyl ring. Most preferably, the phenyl ring is according to structure (BM-7). The substitution pattern of the phenyl ring may be of any regiochemistry, such as 1,2,3-substituted phenyl rings, 1,2,4-substituted phenyl rings, or 1,3,5-substituted phenyl rings. To allow optimal flexibility and conformational freedom, it is preferred that the phenyl ring is according to structure (BM-7), most preferably the phenyl ring is 1,3,5-substituted. The same holds for the pyridine ring of (BM-9).

In a preferred embodiment, the branching moiety BM is selected from a carbon atom, a 40 nitrogen atom, a phosphorus atom, a (hetero)aromatic ring, a (hetero)cycle or a polycyclic moiety.

### Linkers

Each of L¹, L² and L³ may be absent or present, but preferably all three linking units are present. In a preferred embodiment, each of L¹, L² and L³, if present, is independently a chain of at least 2, preferably 5 to 100, atoms selected from C, N, O, S and P. Herein, the chain of atoms refers to the shortest chain of atoms going from the extremities of the linking unit. The atoms within the chain may also be referred to as backbone atoms. As the skilled person will appreciate, atoms having more than two valencies, such as C, N and P, may be appropriately functionalized in order to complete the valency of these atoms. In other words, the backbone atoms are optionally functionalized. In a preferred embodiment, each of L¹, L² and L³, if present, is independently a chain of at least 5 to 50, preferably 6 to 25 atoms selected from C, N, O, S and P. The backbone atoms are preferably selected from C, N and O.

Linkers L¹ and L² connect BM with reactive moieties Q or with connecting groups Z. It is 15 preferred that L¹ and L² are both present, i.e. a = b = 1, more preferably they are the same. In an especially preferred embodiment, (L¹)ₐ-Z is identical to (L²)_{b}-Z, and (L¹)ₐ-Q is identical to (L¹)_{b}-Q.

L² and L² may be independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, L¹ and L², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

Even more preferably, L¹ and L², if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

Yet even more preferably, L¹ and L², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR³, preferably O, wherein R³ is independently selected from the group consisting of hydrogen and C₁ - C₄ alkyl groups, preferably hydrogen or methyl.

Most preferably, L¹ and L², if present, are independently selected from the group consisting of linear or branched C₁-C₂₀ alkylene groups, the alkylene groups being optionally substituted and optionally interrupted by one or more heteroatoms selected from the group of O, S and NR³, wherein R³ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR³, preferably O and/or or S—S, wherein R³ is independently selected from the group consisting of hydrogen and C₁ - C₄ alkyl groups, preferably hydrogen or methyl.

Preferred linkers L² and L² include -(CH₂)ₙ₁-, -(CH₂CH₂)ₙ₁-, -(CH₂CH₂O)ₙ₁-, -(OCH₂CH₂)ₙ₁-, -(CH₂CH₂O)ₙ₁CH₂CH₂-, -CH₂CH₂(OCH₂CH₂)ₙ₁-, -(CH₂CH₂CH₂O)ₙ₁-, -(OCH₂CH₂CH₂)ₙ₁-, -(CH₂CH₂CH₂O)ₙ₁CH₂CH₂CH₂- and -CH₂CH₂CH₂(OCH₂CH₂CH₂)ₙ₁-, wherein n1 is an integer in the range of 1 to 50, preferably in the range of 1 to 40, more preferably in the range of 1 to 30, even more preferably in the range of 1 to 20 and yet even more preferably in the range of 1 to 15. More preferably n1 is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

In one embodiment, L³ is absent and c = 0. In an alternative and more preferred 10 embodiment, L³ is present and c = 1. If L³ is present, it may be the same as L¹ and L² or different, preferably it is different.

In a preferred embodiment, L³ may contain one or more of L⁴, L⁵, L⁶ and L⁷. Thus, in one embodiment, L³ is —(L⁴)ₙ—(L⁵)ₒ—(L⁶)ₚ—(L⁷)_{q}—, wherein L⁴, L⁵, L⁶ and L⁷ are linkers that together form linker L as further defined here below; n, o, p and q are individually 0 or 1. In a preferred embodiment, at least linkers L⁴ and L⁵ are present (i.e. n = 1; o = 1; p = 0 or 1; q = 0 or 1), more preferably linkers L⁴, L⁵ and L⁶ are present and L⁷ is either present or not (i.e. n = 1; o = 1; p = 1; q = 0 or 1). In one embodiment, linkers L⁴, L⁵, L⁶ and L⁷ are present (i.e. n = 1; o = 1; p = 1; q = 1). In one embodiment, linkers L⁴, L⁵ and L⁶ are present and L⁷ is not (i.e. n = 1; o = 1; p = 1; q = 0). In one embodiment n + o + p + q = 1, 2, 3 or 4, preferably 2, 3 or 4, more preferably 3 or 4. In a preferred embodiment, L⁵ and L⁶ are both present, i.e. o + p = 2. Most preferably, n + o + p + q = 4.

Linker L³ may contain a connecting group Z³ that is formed when payload D is connected to the linker construct, which may either be before or after reaction of the linker construct (in particular reactive moieties Q) with a functionalized antibody (in particular reactive moieties F). The connecting group within linker L³ may be formed at the junction any of the linking units L⁴, L⁵, L⁶ and L⁷, or may separately be present within linker L³. For example, L³ may be represented by —Z³—(L⁴)ₙ—(L⁵)ₒ—(L⁶)ₚ—(L⁷)_{q}— or —(L⁴)ₙ—Z³—(L⁵)ₒ—(L⁶)ₚ—(L⁷)_{q}—. Herein, Z may take any form, and is preferably as defined further below for the connecting group obtained by the reaction of Q and F.

### Linker L⁴

Linker L⁴ is either absent (n = 0) or present (n = 1). Preferably, linker L⁴ is present and n = 1. L⁴ may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR¹⁵, wherein y is 0, 1 or 2, preferably y = 2, and R¹⁵ is independently selected from the group consisting of hydrogen, halogen, C₁ - C₂₄ alkyl groups, C₆- C₂₄ (hetero)aryl groups, C₇ - C₂₄ alkyl(hetero)aryl groups and C₇ - C₂₄ (hetero)arylalkyl groups.

L₄ may contain (poly)ethylene glycoldiamines (e.g. 1,8-diamino-3,6-dioxaoctane or equivalents comprising longer ethylene glycol chains), polyethylene glycol or polyethylene oxide chains, polypropylene glycol or polypropylene oxide chains and 1,z-diaminoalkanes wherein z is the number of carbon atoms in the alkane (z may for example be an integer in the range of 1 - 10).

In a preferred embodiment, Linker L₄ comprises an ethylene glycol group, a carboxylic acid moiety, a sulfonate moiety, a sulfone moiety, a phosphate moiety, a phosphinate moiety, an amino group, an ammonium group or a sulfamide group.

In a preferred embodiment, Linker L₄ comprises a sulfamide group, preferably a sulfamide group according to structure (23):

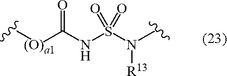

The wavy lines represent the connection to the remainder of the compound, typically to BM and L₅, L₆, L₇ or D, preferably to BM and L₅. Preferably, the (O)ₐC(O) moiety is connected to BM and the NR₁₃ moiety to L₅, L₆, L₇ or D, preferably to L₅.

In structure (23), a1 = 0 or 1, preferably a1 = 1, and R₁₃ is selected from the group consisting of hydrogen, C₁ - C₂₄ alkyl groups, C₃ - C₂₄ cycloalkyl groups, C₂ - C₂₄ (hetero)aryl groups, C₃ -C₂₄ alkyl(hetero)aryl groups and C₃ - C₂₄ (hetero)arylalkyl groups, the C₁ - C₂₄ alkyl groups, C₃ -C₂₄ cycloalkyl groups, C₂ - C₂₄ (hetero)aryl groups, C₃ - C₂₄ alkyl(hetero)aryl groups and C₃ - C₂₄ (hetero)arylalkyl groups optionally substituted and optionally interrupted by one or more heteroatoms selected from O, S and NR₁₄ wherein R₁₄ is independently selected from the group consisting of hydrogen and C₁ - C₄ alkyl groups.

Alternatively, R₁₃ is D connected to N, possibly via a spacer moiety. In one embodiment, 25 this connection is via spacer moiety Sp₂ as defined below, preferably D is connected to N via -(B)ₑ₁-(A)_{f1}-(B)g₁—C(O)— or via —(B)ₑ₁—(A)_{f1}—(B)_{g1}—C(O)—(L₅)ₒ—(L₆)ₚ—(L₇)_{q}—, as further defined below. In another embodiment, R₁₃ is also connected to the first instance of payload D, such that a cyclic structure is formed. For example, N is part of a piperazine moiety, which is connected to D via a carbon atom or nitrogen atom, preferably via the second nitrogen atom of the piperazine ring. Preferably, the cyclic structure, e.g. the piperazine ring, is connected to D via -(B)ₑ₁-(A)_{f1}-(B)_{g1}—C(O)— or via —(B)ₑ₁—(A)_{f1}—(B)_{g1}—C(O)—(L₅)ₒ—(L₆)ₚ—(L₇)_{q}—, as further defined below.

In a preferred embodiment, R₁₃ is hydrogen or a C₁ - C₂₀ alkyl group, more preferably R₁₃ is hydrogen or a C₁ - C₁₆ alkyl group, even more preferably R₁₃ is hydrogen or a C₁ - C₁₀ alkyl group, wherein the alkyl group is optionally substituted and optionally interrupted by one or more heteroatoms selected from O, S and NR₁₄, preferably O, wherein R₁₄ is independently selected from the group consisting of hydrogen and C₁ - C₄ alkyl groups. In a preferred embodiment, R₁₃ is hydrogen. In another preferred embodiment, R₁₃ is a C₁ - C₂₀ alkyl group, more preferably a C₁ -C₁₆ alkyl group, even more preferably a C₁ - C₁₀ alkyl group, wherein the alkyl group is optionally interrupted by one or more O-atoms, and wherein the alkyl group is optionally substituted with an —OH group, preferably a terminal —OH group. In this embodiment it is further preferred that R₁₃ is a (poly)ethylene glycol chain comprising a terminal —OH group. In another preferred embodiment, R₁₃ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R₁₃ is hydrogen or methyl, and most preferably R₁₃ is hydrogen.

In a preferred embodiment, L₄ is according to structure (24):

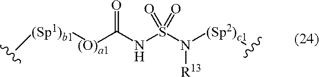

Herein, a and R₁₃ are as defined above, Sp₁ and Sp₂ are independently spacer moieties and b1 and c1 are independently 0 or 1. Preferably, b1 = 0 or 1 and c1 = 1, more preferably b1 = 0 and c1 = 1. In one embodiment, spacers Sp₁ and Sp₂ are independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR₁₆, wherein R₁₆ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp₁ and Sp₂, if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR₁₆, wherein R₁₆ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

Even more preferably, spacer moieties Sp₁ and Sp₂, if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR₁₆, wherein R₁₆ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

Yet even more preferably, spacer moieties Sp₁ and Sp₂, if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR₁₆, wherein R₁₆ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR₁₆, preferably O, wherein R₁₆ is independently selected from the group consisting of hydrogen and C₁ - C₄ alkyl groups, preferably hydrogen or methyl.

Most preferably, spacer moieties Sp₁ and Sp₂, if present, are independently selected from the group consisting of linear or branched C₁-C₂₀ alkylene groups, the alkylene groups being optionally substituted and optionally interrupted by one or more heteroatoms selected from the group of O, S and NR₁₆, wherein R₁₆ is independently selected from the group consisting of hydrogen, C₁ - C₂₄ alkyl groups, C₂ - C₂₄ alkenyl groups, C₂ - C₂₄ alkynyl groups and C₃ - C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR₁₆, preferably O and/or or S—S, wherein R₃ is independently selected from the group consisting of hydrogen and C₁ - C₄ alkyl groups, preferably hydrogen or methyl.

Preferred spacer moieties Sp₁ and Sp₂ thus include —(CH₂)ᵣ₋, —(CH₂CH₂)ᵣ—, —(CH₂CH20)ᵣ—, —(OCH₂CH₂)r—, —(CH₂CH₂O)ᵣCH₂CH₂—, —CH₂CH₂(OCH₂CH₂)ᵣ—, —(CH₂CH₂CH₂O)ᵣ—, —(OCH₂CH₂CH₂)ᵣ—, —(CH₂CH₂CH₂O)ᵣCH₂CH₂CH₂— and, wherein r is an integer in the range of 1 to 50, preferably in the range of 1 to 40, more preferably in the range of 1 to 30, even more preferably in the range of 1 to 20 and yet even more preferably in the range of 1 to 15. More preferably r is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

Alternatively, preferred linkers L₄ may be represented by -(W)ₖ₁-(A)_{d1}-(B)ₑ₁-(A)_{f1}-(C(O))_{g1}-, wherein:
d1 = 0 or 1, preferably d1 = 1;
e1 = an integer in the range 0 10, preferably e1 = 0, 1, 2, 3, 4, 5 or 6, preferably an integer in the range 1 10, most preferably e1 = 1, 2, 3 or 4;
f1 = 0 or 1, preferably f1 = 0;
wherein d1 + e1 + f1 is at least 1, preferably in the range 1 5; and preferably wherein d1 + f1 is at least 1, preferably d1 + f1 = 1.
g1 = 0 or 1, preferably g1 = 1;
k1 = 0 or 1, preferably k1 = 1;
A is a sulfamide group according to structure (23);
B is a —CH₂—CH₂—O— or a —O—CH₂—CH₂— moiety, or (B)ₑ₁ is a —(CH₂—CH₂—O)ₑ₃—CH₂—CH₂—moiety, wherein e3 is defined the same way as e1;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or —(4—Ph)CH₂NHC(O)(CH₂)ₘC(O)NH—, preferably
   wherein W is —OC(O)NH—, —C(O)(CH₂)ₘC(O)NH— or —C(O)NH—, and wherein m is an integer in the range 0 - 10, preferably m = 0, 1, 2, 3, 4, 5 or 6, most preferably m = 2 or 3;
preferably wherein L₄ is connected to BM via (A)_{d1}-(B)ₑ₁ and to (L₅)ₒ via (C(O))_{g1}, preferably via C(O).

In the context of the present embodiment, the wavy lines in structure (23) represent the connection to the adjacent groups such as (W)ₖ₁, (B)ₑ₁ and (C(O))_{g1}. It is preferred that A is according to structure (23), wherein a1 = 1 and R₁₃ = H or a C₁ - C₂₀ alkyl group, more preferably R₁₃ = H or methyl, most preferably R₁₃ = H.

Preferred linkers L₄ are as follows:
(a) k1 = 0; d1 = 1; g1 = 1; f1 = 0; B = —CH₂—CH₂—O—; e1 = 1, 2, 3 or 4, preferably e1 = 2.
(b) k1 = 1; W═ —C(O)(CH₂)ₘC(O)NH—; m = 2; d1 = 0; (B)ₑ₁ = —(CH₂—CH₂—O)ₑ₃—CH₂—CH₂—; f1 = 0; g1 = 1; e3 = 1, 2, 3 or 4, preferably e1 = 1.
(c) k1 = 1; W = —OC(O)NH—; d1 = 0; B═ —CH₂—CH₂—O—; g1 = 1; f1 = 0; e1 = 1, 2, 3 or 4, preferably e1 = 2.
(d) k1 = 1; W = —C(O)(CH₂)ₘC(O)NH—; m = 2; d1 = 0; (B)ₑ₁ = —(CH₂—CH₂—O)ₑ₃—CH₂—CH₂—; f1 = 0; g1 = 1; e3 = 1, 2, 3 or 4, preferably e3 = 4.
(e) k1 = 1; W = —OC(O)NH—; d1 = 0; (B)ₑ₁ = —(CH₂—CH₂—O)ₑ₃—CH₂—CH₂—; g1 = 1; f1 = 0; e3 = 1, 2, 3 or 4, preferably e3 = 4.
(f) k1 = 1; W = —(4—Ph)CH₂NHC(O)(CH₂)ₘC(O)NH—, m = 3; d1 = 0; (B)ₑ₁ = —(CH₂—CH₂—O)e3—CH₂—CH₂—; g1 = 1; f1 = 0; e3 = 1, 2, 3 or 4, preferably e3 = 4.
(g) k1 = 0; d1 = 0; g1 = 1; f1 = 0; B = —CH₂—CH₂—O—; e1 =1, 2, 3 or 4, preferably e1 = 2.
(h) k1 = 1; W = —C(O)NH—; d1 = 0; g1 = 1; f1 = 0; B = —CH₂—CH₂—O—; e1 = 1, 2, 3 or 4, preferably e1 = 2.

In one embodiment, linker L₄ comprises a branching nitrogen atom, which is located in the backbone between BM and (L₅)ₒ and which contains a further moiety D as substituent, which is 10 preferably linked to the branching nitrogen atom via a linker. An example of a branching nitrogen atom is the nitrogen atom NR₁₃ in structure (23), wherein R₁₃ is connected to a second occurrence of D via a spacer moiety. Alternatively, a branching nitrogen atoms may be located within L₄ according to structure -(W)ₖ₁-(A)_{d1}-(B)ₑ₁-(A)_{f1}-(C(O))_{g1}-. In one embodiment, L₄ is represented by -(W)ₖ₁-(A)_{d1}-(B)ₑ₁-(A)_{f1}-(C(O))_{g1}-N*[-(A)_{d1}-(B)ₑ₁-(A)_{f1}-(C(O))_{g1}-]₂, wherein A, B, W, d1, e1, f1, g1 and k1 are as defined above and individually selected for each occurrence, and N* is the branching nitrogen atoms, to which two instances of -(A)_{d1}-(B)ₑ₁-(A)_{f1}-(C(O))_{g1}- are connected. Herein, both (C(O))_{g1} moieties are connected to —(L₅)ₒ—(L₆)ₚ—(L₇)_{q}—D, wherein L₅, L₆, L₇, o, p, q and D are as defined above and are each selected individually. In a most preferred embodiment, such a branching atom is not present and linker L₄ does not contain a connection to a further moiety D.

### Linker L₅

Linker L₅ is either absent (o = 0) or present (o = 1). Preferably, linker L₅ is present and o = 1. Linker L₅ is a peptide spacer as known in the art, preferably comprising 2 - 5 amino acids, more preferably a dipeptide or tripeptide spacer, most preferably a dipeptide spacer. Although any peptide spacer may be used, preferably linker L₅ is selected from Val-Cit, Val-Ala, Val-Lys, Val-Arg, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, more preferably Val-Cit, Val-Ala, Ala-Ala-Asn. In one embodiment, L₅ = Val-Cit. In one embodiment, L₅ = Val-Ala.

In a preferred embodiment, L₅ is represented by general structure (27):

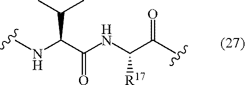

Herein, R₁₇ = CH₃ or CH₂CH₂CH₂NHC(O)NH₂. The wavy lines indicate the connection to (L₄)ₙ and (L⁶)ₚ, preferably L⁵ according to structure (27) is connected to (L⁴)ₙ via NH and to (L⁶)ₚ via C(O).

### Linker L⁶

Linker L⁶ is either absent (p = 0) or present (p = 1). Preferably, linker L⁶ is present and p = 1. Linker L⁶ is a self-cleavable spacer, also referred to as self-immolative spacer. Preferably, L⁶ is para-aminobenzyloxycarbonyl (PABC) derivative, more preferably a PABC derivative according to structure (25).

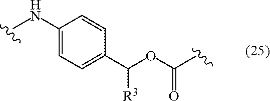

Herein, the wavy lines indicate the connection to (L₅)ₙ and to (L₇)ₚ. Typically, the PABC derivative is connected via NH to (L₅)ₙ, and via O to (L₇)ₚ.

R₃ is H, R₄ or C(O)R₄, wherein R₄ is C₁ - C₂₄ (hetero)alkyl groups, C₃ - C₁₀ (hetero)cycloalkyl groups, C₂ - C₁₀ (hetero)aryl groups, C₃ - C₁₀ alkyl(hetero)aryl groups and C₃ -C₁₀ (hetero)arylalkyl groups, which optionally substituted and optionally interrupted by one or more heteroatoms selected from O, S and NR₅ wherein R₅ is independently selected from the group consisting of hydrogen and C₁ - C₄ alkyl groups. Preferably, R₄ is C₃ - C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH20)ₛH. The polyalkylene glycol is most preferably a polyethylene glycol, preferably —(CH₂CH₂O)ₛH, wherein s is an integer in the range 1 - 10, preferably 1 - 5, most preferably s = 1, 2, 3 or 4. More preferably, R₃ is H or C(O)R₄, wherein R₄ = 4-methyl-piperazine or morpholine. Most preferably, R₃ is H.

### Linker L₇

Linker L₇ is either absent (q = 0) or present (q = 1). Preferably, linker L₇ is present and q = 1. Linker L₇ is an aminoalkanoic acid spacer, i.e. —N—(Ch—alkylene)—C(O)—, wherein h is an integer in the range 1 to 20, preferably 1 - 10, most preferably 1 - 6. Herein, the aminoalkanoic acid spacer is typically connected to L₆ via the nitrogen atom and to D via the carbonyl moiety. Preferred linkers L₇ are selected from 6-aminohexanoic acid (Ahx, h = 6), β-alanine (h = 2) and glycine (Gly, h = 1), even more preferably 6-aminohexanoic acid or glycine. In one embodiment, L₇= 6-aminohexanoic acid. In one embodiment, L₇= glycine. Or linker L₇ is a an ethyleneglycol spacer according to the structure —N—(CH₂—CH₂—O)ₑ₆—(CH₂)ₑ₇—(C(O)—, wherein e6 is an integer in the range 1 - 10 and e7 is an integer in the range 1 - 3.

### Payload D

In a preferred embodiment of the linker-conjugate according to the invention, the payload is selected from the group consisting of an active substance, a reporter molecule, a polymer, a solid surface, a hydrogel, a nanoparticle, a microparticle and a biomolecule. Especially preferred payloads are active substances and reporter molecules, in particular active substances.

The term “active substance” herein relates to a pharmacological and/or biological substance, i.e. a substance that is biologically and/or pharmaceutically active, for example a drug, a prodrug, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine.

When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterials agents, peptides and oligonucleotides. Examples of cytotoxins include colchicine, vinca alkaloids, anthracyclines, camptothecins, doxorubicin, daunorubicin, taxanes, calicheamycins, tubulysins, irinotecans, an inhibitory peptide, amanitin, deBouganin, duocarmycins, maytansines, auristatins, enediynes, pyrrolobenzodiazepines (PBDs) or indolinobenzodiazepine dimers (IGN) or PNU159,682.

The term “reporter molecule” herein refers to a molecule whose presence is readily detected, for example a diagnostic agent, a dye, a fluorophore, a radioactive isotope label, a contrast agent, a magnetic resonance imaging agent or a mass label.

A wide variety of fluorophores, also referred to as fluorescent probes, is known to a person skilled in the art. Several fluorophores are described in more detail in e.g. G.T. Hermanson, “Bioconjugate Techniques”, Elsevier, 3_{rd} Ed. 2013, Chapter 10: “Fluorescent probes”, p. 395 - 463, incorporated by reference. Examples of a fluorophore include all kinds of Alexa Fluor (e.g. Alexa Fluor 555), cyanine dyes (e.g. Cy3 or Cy5) and cyanine dye derivatives, coumarin derivatives, fluorescein and fluorescein derivatives, rhodamine and rhodamine derivatives, boron dipyrromethene derivatives, pyrene derivatives, naphthalimide derivatives, phycobiliprotein derivatives (e.g. allophycocyanin), chromomycin, lanthanide chelates and quantum dot nanocrystals.

Examples of a radioactive isotope label include ^{99m}Tc, ¹¹¹In, ^{114m}In, ¹¹⁵In,¹⁸F, ¹⁴C, ⁶⁴Cu, ¹³¹I, ¹²⁵I, ¹²³I, ²¹²Bi, ⁸⁸Y, ⁹⁰Y, ⁶⁷Cu, ¹⁸⁶Rh, ¹⁸⁸Rh, ⁶⁶Ga, ⁶⁷Ga and ¹⁰B, which is optionally connected via a chelating moiety such as e.g. DTPA (diethylenetriaminepentaacetic anhydride), DOTA (1,4,7,10-tetraazacyclododecane-N,N′,N″,N‴-tetraacetic acid), NOTA (1,4,7-triazacyclononane N,N′,N″-triacetic acid), TETA (1,4,8,11-tetraazacyclotetradecane-N,N′,N″,N‴-tetraacetic acid), DTTA (N₁-(ₚ-isothiocyanatobenzyl)-diethylenetriamine-N₁,N₂,N₃,N₃-tetraacetic acid), deferoxamine or DFA (N′-[5-[[4-[[5-(acetylhydroxyamino)pentyl]amino]-1,4-dioxobutyl]hydroxyamino]pentyl]—N—(5-aminopentyl)-N-hydroxybutanediamide) or HYNIC (hydrazinonicotinamide). Isotopic labelling techniques are known to a person skilled in the art, and are described in more detail in e.g. G.T. Hermanson, “Bioconjugate Techniques”, Elsevier, 3^{rd} Ed. 2013, Chapter 12: “Isotopic labelling techniques”, p. 507 - 534, incorporated by reference.

Polymers suitable for use as a payload D in the compound according to the invention are known to a person skilled in the art, and several examples are described in more detail in e.g. G.T. Hermanson, “Bioconjugate Techniques”, Elsevier, 3rd Ed. 2013, Chapter 18: “PEGylation and synthetic polymer modification”, p. 787 - 838, incorporated by reference. When payload D is a polymer, payload D is preferably independently selected from the group consisting of a poly(ethyleneglycol) (PEG), a polyethylene oxide (PEO), a polypropylene glycol (PPG), a polypropylene oxide (PPO), a 1,x-diaminoalkane polymer (wherein x is the number of carbon atoms in the alkane, and preferably x is an integer in the range of 2 to 200, preferably 2 to 10), a (poly)ethylene glycol diamine (e.g. 1,8-diamino-3,6-dioxaoctane and equivalents comprising longer ethylene glycol chains), a polysaccharide (e.g. dextran), a poly(amino acid) (e.g. a poly(L-lysine)) and a poly(vinyl alcohol).

Solid surfaces suitable for use as a payload D are known to a person skilled in the art. A solid surface is for example a functional surface (e.g. a surface of a nanomaterial, a carbon nanotube, a fullerene or a virus capsid), a metal surface (e.g. a titanium, gold, silver, copper, nickel, tin, rhodium or zinc surface), a metal alloy surface (wherein the alloy is from e.g. aluminium, bismuth, chromium, cobalt, copper, gallium, gold, indium, iron, lead, magnesium, mercury, nickel, potassium, plutonium, rhodium, scandium, silver, sodium, titanium, tin, uranium, zinc and/or zirconium), a polymer surface (wherein the polymer is e.g. polystyrene, polyvinylchloride, polyethylene, polypropylene, poly(dimethylsiloxane) or polymethylmethacrylate, polyacrylamide), a glass surface, a silicone surface, a chromatography support surface (wherein the chromatography support is e.g. a silica support, an agarose support, a cellulose support or an alumina support), etc. When payload D is a solid surface, it is preferred that D is independently selected from the group consisting of a functional surface or a polymer surface.

Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. When the payload is a hydrogel, it is preferred that the hydrogel is composed of poly(ethylene)glycol (PEG) as the polymeric basis.

Micro- and nanoparticles suitable for use as a payload D are known to a person skilled in the art. A variety of suitable micro- and nanoparticles is described in e.g. G.T. Hermanson, “Bioconjugate Techniques”, Elsevier, 3^{rd} Ed. 2013, Chapter 14: “Microparticles and nanoparticles”, p. 549 - 587, incorporated by reference. The micro- or nanoparticles may be of any shape, e.g. spheres, rods, tubes, cubes, triangles and cones. Preferably, the micro- or nanoparticles are of a spherical shape. The chemical composition of the micro- and nanoparticles may vary. When payload D is a micro- or a nanoparticle, the micro- or nanoparticle is for example a polymeric micro-or nanoparticle, a silica micro- or nanoparticle or a gold micro- or nanoparticle. When the particle is a polymeric micro- or nanoparticle, the polymer is preferably polystyrene or a copolymer of styrene (e.g. a copolymer of styrene and divinylbenzene, butadiene, acrylate and/or vinyltoluene), polymethylmethacrylate (PMMA), polyvinyltoluene, poly(hydroxyethyl methacrylate (pHEMA) or poly(ethylene glycol dimethacrylate/2-hydroxyethylmetacrylae) [poly(EDGMA/HEMA)]. Optionally, the surface of the micro- or nanoparticles is modified, e.g. with detergents, by graft polymerization of secondairy polymers or by covalent attachment of another polymer or of spacer moieties, etc.

Payload D may also be a biomolecule. Biomolecules, and preferred embodiments thereof, are described in more detail below. When payload D is a biomolecule, it is preferred that the biomolecule is selected from the group consisting of proteins (including glycoproteins and antibodies), polypeptides, peptides, glycans, lipids, nucleic acids, oligonucleotides, polysaccharides, oligosaccharides, enzymes, hormones, amino acids and monosaccharides.

The DAR1 antibody-payload conjugates according to the present invention are especially suitable to be used with highly potent cytotoxins, such as PBD dimers, indolinobenzodiazepine dimers (IGN), enediynes, PNU159,682, duocarmycin dimers, amanitin and auristatins, preferably PBD dimers, indolinobenzodiazepine dimers (IGN), enediynes or PNU159,682. In an especially preferred embodiment, the payload is selected form the group of PBD dimers, indolinobenzodiazepine dimers (IGN), enediynes, PNU159,682, duocarmycin dimers, amanitin and auristatins, preferably PBD dimers, indolinobenzodiazepine dimers (IGN), enediynes or PNU159,682. In a further preferred embodiment, the payload is not a symmetric or dimeric payload.

In a preferred embodiment, the antibody-payload conjugate according to the invention is according to structure (5). The conjugate according to this embodiment comprises (G)ₑ and Su, which are further defined here below.

Each of the two GlcNAc moieties in (4) are preferably present at a native N-glycosylation site in the Fc-fragment of antibody AB. Preferably, said GlcNAc moieties are attached to an asparagine amino acid in the region 290-305 of AB. In a further preferred embodiment, the antibody is an IgG type antibody, and, depending on the particular IgG type antibody, said GlcNAc moieties are present on amino acid asparagine 297 (Asn297 or N297) of the antibody.

G is a monosaccharide moiety and e is an integer in the range of 0 - 10. G is preferably selected from the group consisting of glucose (Glc), galactose (Gal), mannose (Man), fucose (Fuc), N-acetylglucosamine (GlcNAc), N-acetylgalactosamine (GalNAc), N-acetylneuraminic acid (NeuNAc) and sialic acid and xylose (Xyl). More preferably, G is selected from the group consisting of glucose (Glc), galactose (Gal), mannose (Man), fucose (Fuc), N-acetylglucosamine (GlcNAc) and N-acetylgalactosamine (GalNAc).

In a preferred embodiment, e is 0 and G is absent. G is typically absent when the glycan of the antibody is trimmed. Trimming refers to treatment with endoglycosidase, such that only the core GlcNAc moiety of the glycan remains.

In another preferred embodiment, e is an integer in the range of 1 - 10. In this embodiment it is further preferred that G is selected from the group consisting of glucose (Glc), galactose (Gal), mannose (Man), fucose (Fuc), N-acetylglucosamine (GlcNAc), N-acetylgalactosamine (GalNAc), N-acetylneuraminic acid (NeuNAc) or sialic acid and xylose (Xyl), more preferably from the group consisting of glucose (Glc), galactose (Gal), mannose (Man), fucose (Fuc), N-acetylglucosamine (GlcNAc) and N-acetylgalactosamine (GalNAc).

When e is 3 - 10, (G)ₑ may be linear or branched. Preferred examples of branched oligosaccharides (G)ₑ are (a), (b), (c), (d), (e), (f), (h) and (h) as shown below.

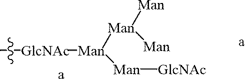

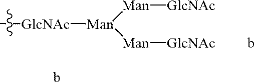

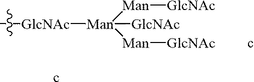

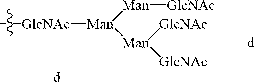

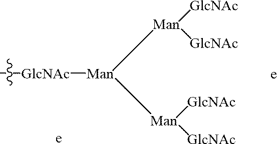

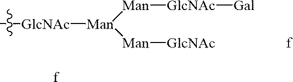

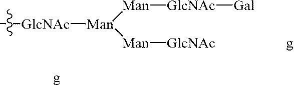

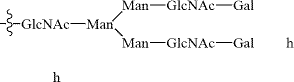

In case G is present, it is preferred that it ends in GlcNAc. In other words, the monosaccharide residue directly connected to Su is GlcNAc. The presence of a GlcNAc moiety facilitates the synthesis of the functionalized antibody, as monosaccharide derivative Su can readily be introduced by glycosyltransfer onto a terminal GlcNAc residue. In the above preferred embodiments for (G)ₑ, having structure (a) - (h), moiety Su may be connected to any of the terminal GlcNAc residues, i.e. not the one with the wavy bond, which is connected to the core GlcNAc residue on the antibody.

It is particularly preferred that G is absent, i.e. that e = 0. An advantage of an antibody-payload conjugate (1) wherein e = 0 is that when such conjugate is used clinically, binding to Fc gamma receptors CD16, CD32 and CD64 is significantly reduced or fully abrogated.

Su is a monosaccharide derivative, also referred to as sugar derivative. Preferably, the sugar derivative is able to be incorporated into the functionalized antibody by means of glycosyltransfer. See FIG. 2 for some preferred examples of nucleotide-sugar derivatives that can be introduced. More preferably, Su is Gal, Glc, GalNAc or GlcNAc, more preferably Gal or GalNAc, most preferably GalNAc. The term derivative refers to the monosaccharide being appropriately functionalized in order to connect to (G)ₑ and F.

### Preparation Method

The present invention also relates to a method for preparing an antibody-payload conjugate having a hypothetical payload-to-antibody ratio of 1, comprising the steps of:
(a) reacting a compound having structure (2) containing at least two reactive groups Q with an antibody having structure (3), which is functionalized with two reactive groups F:

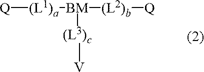

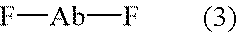

wherein:
   Ab is an antibody;
   a, b and c are each individually 0 or 1;
   L¹, L² and L³ are linkers;
   V is a reactive group Q′ or a payload D;
   BM is a branching moiety;
   Q and F are reactive groups capable of undergoing a cycloaddition reaction wherein they are joined in connecting group Z;
   to obtain a functionalized antibody according to structure (1′):

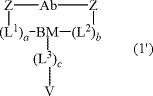

wherein Z is a connecting group obtained by the reaction of Q with F;
wherein the functionalized antibody according to structure (1′) is the antibody-payload conjugate in case V is the payload D;
or the functionalized antibody according to structure (1′) is further reacted according to step (b) in case V is a reactive group Q′ to obtain the antibody-payload conjugate in case V is the payload D;
(b) in case V = Q′, reacting reactive group Q′ with a payload containing a reactive group F′ to obtain the antibody-payload conjugate wherein V is the payload D.

In a preferred embodiment, antibody having structure (3) has structure (3b):

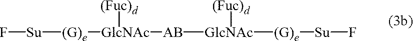

In a preferred embodiment, functionalized antibody according to structure (1) has structure (5b):

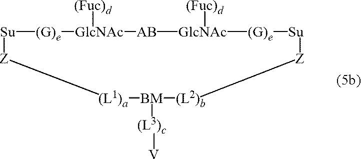

The method according to the present invention can take two major forms, one wherein step (b) is not performed and one wherein step (b) is performed.

In one embodiment, step (b) is not performed and V present on the compound having structure (2) is the payload D. In that case, step (a) affords the final conjugate (structure (1)) directly. The process according to this preferred embodiment can be represented according to Scheme 1.

### Scheme 1

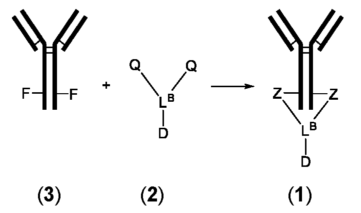

Herein, L^{B} represents the trivalent linker according to structure (9), and which is further defined above.

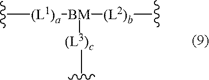

Thus, in a preferred embodiment, a functionalized antibody according to structure (1) is obtained in step (a), wherein D is the payload, and step (b) is not performed.

In one embodiment, step (b) is performed and V present on the compound having structure (2) is a reactive group Q′. In that case, step (a) affords an intermediary functionalized antibody having structure (1) wherein V = Q′ (here below depicted as (1b)). This intermediary functionalized antibody contains a further reactive group Q′, which is reacted with an appropriately functionalized payload with reactive group F, to obtain the final conjugate having structure (1) wherein V = D. The process according to this preferred embodiment can be represented according to Scheme 2.

### Scheme 2

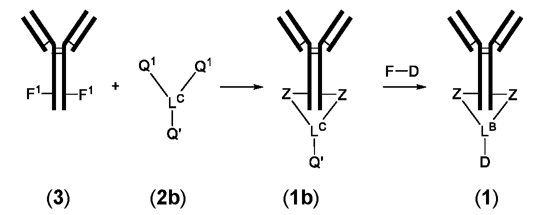

Herein, Q¹ and F¹ are reactive moieties just as Q and F, and the definition and preferred embodiments of Q and F equally apply to Q¹ and F¹. The presence of Q′ in the linker compound (2) should not interfere with the reaction, which can be accomplished with the inertness of Q′ in the reaction between Q¹ and F¹. The inventors have found that a trivalent linker compound wherein both Q¹ and Q′ are the same reactive moiety, the reaction with Ab(F¹)₂ only occurs for two combinations Q¹/Q′, and the third reactive moiety remains unreacted. Further reduction of a third reaction taking place at the linker compound is accomplished by performing the reaction in dilute conditions.

Thus, in a preferred embodiment, a functionalized antibody according to structure (1′) is obtained in step (a), wherein V is a reactive group Q′, i.e. structure (1b), and step (b) is performed.

The “payload-to-antibody ratio”, also known as drug-to-antibody ratio (DAR), refers to the ratio of payload molecules to antibody molecules in a conjugate. The present invention provides an efficient route towards conjugates having a DAR of 1, i.e. one payload molecule is conjugated to one antibody molecule. The payload-to-antibody ratio of the product may be slightly below the hypothetical payload-to-antibody ratio, since not all functionalized antibodies may react with the linker compound of structure (2), such that the actual payload-to-antibody ratio may deviate somewhat (i.e. may be somewhat lower) from the hypothetical payload-to-antibody ratio. The process according to the present invention provides product mixtures with a payload-to-antibody ratio close to the hypothetical ratio of 1.

The present invention provides a greatly improved method for preparing antibody conjugates having a payload-to-antibody ratio of 1, when compared to conventional methods. Conventional methods struggle with introduction of only a single attachment point in the antibody. Antibodies contain many amino acids, such that random conjugation, such as maleimide-cysteine conjugation, typically gives a broad distribution with conjugates bearing up to 8 or even more payloads. Other conjugation methods suffer from the fact that antibodies are symmetrical, thus providing at least two of any attachment point that could be used. As such, genetic engineering may be relied upon to design recombinant antibodies containing only one attachment point.

An alternative prior art approach involves the use of symmetrically functionalized payloads, wherein a symmetric payload (a dimer) is functionalized symmetrically with two identical reactive moieties, via a linker. These two reactive moieties then react with two attachment points provided in the antibody.

The process according to the invention elegantly converts the two attachment points of an antibody into a single attachment point, by clipping a bifunctional linker compound over the two attachment points on the antibody. As demonstrated in the examples, conjugates having a payload-to-antibody ratio of 1 can elegantly be obtained as such. Also, by virtue of the branching moiety, any payload can be conjugated to the antibody, such that the present process is not limited to symmetrical payloads.

In case V = D, the reaction of step (a) is a conjugation reaction. Otherwise, in case V = Q′, the reaction of step (b) is a conjugation reaction. The process according to the invention is compatible with any conjugation technology, and any such technology can be used for both step (a) and step (b), if performed.

In a preferred embodiment, the reaction of step (a) is a [4+2] cycloaddition or a 1,3-dipolar cycloaddition.

The antibody according to structure (3) may be prepared by any means known in the art. For example, reduction of interchain disulfide bonds of an antibody followed by reaction with a defined number of reactive moiety F containing maleimide constructs (or other thiol-reactive constructs) leads to a loading of groups F that can be tailored by stoichiometry. A more controlled, site-specific process of antibody conjugation can be achieved for example by genetic engineering of the antibody to contain two unpaired cysteines (one per heavy chain or one per light chain), to provide exactly two reactive moieties F onto the antibody upon subjection of the antibody to F containing maleimide constructs. Genetic encoding enables the direct expression of an antibody to contain a predefined number of reactive moieties F at specific sites by applying the AMBER stop codon. A range of enzymatic approaches have been also been reported to install a defined number of reactive moieties F onto an antibody, for example based on transglutaminase (TGase), sortase, formyl-glycine generating enzyme (FGE) and others. Thus, in one embodiment, the functionalized antibody is prepared by reduction of interchain disulfide bonds followed by reaction with F-containing thiol-reactive constructs, introduction of unpaired cysteine residues followed by reaction with F-containing thiol-reactive constructs, enzymatic introduction of reactive moieties F, and introduction of reactive moieties by genetic engineering. The use of genetic engineering is least preferred in the context of the present application, while enzymatic introduction of reactive moieties F is most preferred.

In a preferred embodiment, GlycoConnect technology (see e.g. WO 2014/065661 and Van Geel et al., Bioconj. Chem. 2015, 26, 2233-2242, incorporated by reference) utilizes the naturally present glycans at the heavy chain of monoclonal antibodies to introduce a fixed number of click probes, in particular azides. Thus, in a preferred embodiment, the functionalized antibody is prepared by (i) optionally trimming of the native glycan with a suitable endoglycosidase, thereby liberating the core GlcNAc, which is typically present on Asn-297, followed by (ii) transfer of an unnatural, azido-bearing sugar substrate from the corresponding UDP-sugar under the action of a suitable glycosyltransferase, for example transfer of GalNAz with galactosyltransferase mutant Gal-T(Y289L) or 6-azidoGalNAc with GalNAc-transferase (GalNAc-T). Alternatively, GalNAc-T can also be applied to install onto the core GlcNAc GalNAc derivatives harbouring aromatic moieties or thiol function on the Ac group. The functionalized antibody according to structure (5) can be obtained with this technology, wherein trimming step (i) may be employed to obtained functionalized antibodies having e = 0, or can be omitted to obtain functionalized antibodies having e = 1 - 10. Preferably, step (i) is performed and e = 0.

### Reactive Moieties Q and F

In the context of the present invention, the term “reactive moiety” may refer to a chemical moiety that comprises a functional group, but also to a functional group itself. For example, a cyclooctynyl group is a reactive group comprising a functional group, namely a C—C triple bond. However, a functional group, for example an azido functional group, a thiol functional group or an alkynyl functional group, may herein also be referred to as a reactive group.

In order to be reactive in the process according to the invention, reactive moiety Q should be capable of reacting with reactive moiety F present on the functionalized antibody. In other words, reactive moiety Q is reactive towards reactive moiety F present on the functionalized antibody. Herein, a reactive moiety is defined as being “reactive towards” another reactive moiety when said first reactive moiety reacts with said second reactive moiety selectively, optionally in the presence of other functional groups. Complementary reactive moiety are known to a person skilled in the art, and are described in more detail below and are exemplified in FIG. 1. As such, the conjugation reaction is a chemical reaction between Q and F forming a conjugate comprising a covalent connection between the antibody and the payload. The definition of the reactive moiety Q provided here equally applies to F, Q¹, F¹ and Q′.

In a preferred embodiment, reactive moiety is selected from the group consisting of, optionally substituted, alkenyl groups, alkynyl groups, tetrazinyl groups, azido groups, nitrile oxide groups, nitrone groups, nitrile imine groups, diazo groups, ketone groups, (O-alkyl)hydroxylamino groups, hydrazine groups, allenamide groups, triazine groups, phosphonamidite groups. In an especially preferred embodiment, reactive moiety Q is an azide group or an alkynyl group, most preferably reactive moiety Q is an alkynyl group. In case Q is an alkynyl group, it is preferred that Q is selected from terminal alkyne groups, (hetero)cycloalkynyl groups and bicyclo[6.1.0]non-4-yn-9-yl] groups.

In another preferred embodiment, Q comprises or is an alkenyl group, including cycloalkenyl groups, preferably Q is an alkenyl group. The alkenyl group may be linear or branched, and is optionally substituted. The alkenyl group may be a terminal or an internal alkenyl group. The alkenyl group may comprise more than one C—C double bond, and preferably comprises one or two C-C double bonds. When the alkenyl group is a dienyl group, it is further preferred that the two C—C double bonds are separated by one C—C single bond (i.e. it is preferred that the dienyl group is a conjugated dienyl group). Preferably said alkenyl group is a C₂ - C₂₄ alkenyl group, more preferably a C₂ - C₁₂ alkenyl group, and even more preferably a C₂ - C₆ alkenyl group. It is further preferred that the alkenyl group is a terminal alkenyl group. More preferably, the alkenyl group is according to structure (Q8) as shown below, wherein I is an integer in the range of 0 to 10, preferably in the range of 0 to 6, and p is an integer in the range of 0 to 10, preferably 0 to 6. More preferably, I is 0, 1, 2, 3 or 4, more preferably I is 0, 1 or 2 and most preferably I is 0 or 1. More preferably, p is 0, 1, 2, 3 or 4, more preferably p is 0, 1 or 2 and most preferably p is 0 or 1. It is particularly preferred that p is 0 and I is 0 or 1, or that p is 1 and I is 0 or 1.

A particularly preferred alkenyl group is a cycloalkenyl group, including heterocycloalkenyl groups, wherein the cycloalkenyl group is optionally substituted. Preferably said cycloalkenyl group is a C₃ - C₂₄ cycloalkenyl group, more preferably a C₃ - C₁₂ cycloalkenyl group, and even more preferably a C₃ - C₈ cycloalkenyl group. In a preferred embodiment, the cycloalkenyl group is a trans-cycloalkenyl group, more preferably a trans-cyclooctenyl group (also referred to as a TCO group) and most preferably a trans-cyclooctenyl group according to structure (Q9) or (Q10) as shown below. In another preferred embodiment, the cycloalkenyl group is a cyclopropenyl group, wherein the cyclopropenyl group is optionally substituted. In another preferred embodiment, the cycloalkenyl group is a norbornenyl group, an oxanorbornenyl group, a norbornadienyl group or an oxanorbornadienyl group, wherein the norbornenyl group, oxanorbornenyl group, norbornadienyl group or an oxanorbornadienyl group is optionally substituted. In a further preferred embodiment, the cycloalkenyl group is according to structure (Q11), (Q12), (Q13) or (Q14) as shown below, wherein X⁴ is CH₂ or O, R²⁷ is independently selected from the group consisting of hydrogen, a linear or branched C₁ - C₁₂ alkyl group or a C₄ - C₁₂ (hetero)aryl group, and R¹⁴ is selected from the group consisting of hydrogen and fluorinated hydrocarbons. Preferably, R²⁷ is independently hydrogen or a C₁ - C₆ alkyl group, more preferably R²⁷ is independently hydrogen or a C₁ - C₄ alkyl group. Even more preferably R²⁷ is independently hydrogen or methyl, ethyl, n-propyl, i-propyl, n-butyl, s-butyl or t-butyl. Yet even more preferably R²⁷ is independently hydrogen or methyl. In a further preferred embodiment, R¹⁴ is selected from the group of hydrogen and -CF₃, -C₂F₅, -C₃F₇and -C₄F₉, more preferably hydrogen and -CF₃. In a further preferred embodiment, the cycloalkenyl group is according to structure (Q11), wherein one R²⁷ is hydrogen and the other R²⁷ is a methyl group. In another further preferred embodiment, the cycloalkenyl group is according to structure (Q12), wherein both R²⁷ are hydrogen. In these embodiments it is further preferred that I is 0 or 1. In another further preferred embodiment, the cycloalkenyl group is a norbornenyl (X⁴ is CH₂) or an oxanorbornenyl (X⁴ is O) group according to structure (Q13), or a norbornadienyl (X⁴ is CH₂) or an oxanorbornadienyl (X⁴ is O) group according to structure (Q14), wherein R²⁷ is hydrogen and R¹⁴ is hydrogen or -CF₃, preferably -CF₃.

In another preferred embodiment, Q comprises or is an alkynyl group, including cycloalkynyl groups, preferably Q comprises an alkynyl group. The alkynyl group may be linear or branched, and is optionally substituted. The alkynyl group may be a terminal or an internal alkynyl group. Preferably said alkynyl group is a C₂ - C₂₄ alkynyl group, more preferably a C₂ - C₁₂ alkynyl group, and even more preferably a C₂ - C₆ alkynyl group. It is further preferred that the alkynyl group is a terminal alkynyl group. More preferably, the alkynyl group is according to structure (Q15) as shown below, wherein I is an integer in the range of 0 to 10, preferably in the range of 0 to 6. More preferably, I is 0, 1, 2, 3 or 4, more preferably I is 0, 1 or 2 and most preferably I is 0 or 1.

A particularly preferred alkynyl group is a cycloalkynyl group, including hetero cycloalkynyl group, cycloalkenyl group is optionally substituted. Preferably, the (hetero)cycloalkynyl group is a (hetero)cyclooctynyl group, i.e. a heterocyclooctynyl group or a cyclooctynyl group, wherein the (hetero)cyclooctynyl group is optionally substituted. In a further preferred embodiment, the (hetero)cyclooctynyl group is according to structure (Q36) and defined further below. Preferred examples of the (hetero)cyclooctynyl group include structure (Q16), also referred to as a DIBO group, (Q17), also referred to as a DIBAC group, or (Q18), also referred to as a BARAC group, (Q19), also referred to as a COMBO group, and (Q20), also referred to as a BCN group, all as shown below, wherein X⁵ is O or N R²⁷, and preferred embodiments of R²⁷ are as defined above. The aromatic rings in (Q16) are optionally O-sulfonylated at one or more positions, preferably at two positions, most preferably as in (Q40) (sulfonylated dibenzocyclooctyne (s-DIBO)), whereas the rings of (Q17) and (Q18) may be halogenated at one or more positions. A particularly preferred cycloalkynyl group is a bicyclo[6.1.0]non-4-yn-9-yl] group (BCN group), which is optionally substituted. Preferably, the bicyclo[6.1.0]non-4-yn-9-yl] group is according to structure (Q20) as shown below.

In another preferred embodiment, Q comprises or is a conjugated (hetero)diene group, preferably Q is a conjugated (hetero)diene group capable of reacting in a Diels-Alder reaction. Preferred (hetero)diene groups include optionally substituted tetrazinyl groups, optionally substituted 1,2-quinone groups and optionally substituted triazine groups. More preferably, said tetrazinyl group is according to structure (Q21), as shown below, wherein R²⁷ is selected from the group consisting of hydrogen, a linear or branched C₁ - C₁₂ alkyl group or a C₄ - C₁₂ (hetero)aryl group. Preferably, R²⁷ is hydrogen, a C₁ - C₆ alkyl group or a C₄ - C₁₀ (hetero)aryl group, more preferably R²⁷ is hydrogen, a C₁ - C₄ alkyl group or a C₄ - C₆ (hetero)aryl group. Even more preferably R²⁷ is hydrogen, methyl, ethyl, n-propyl, i-propyl, n-butyl, s-butyl, t-butyl or pyridyl. Yet even more preferably R²⁷ is hydrogen, methyl or pyridyl. More preferably, said 1,2-quinone group is according to structure (Q22) or (Q23). Said triazine group may be any regioisomer. More preferably, said triazine group is a 1,2,3-triazine group or a 1,2,4-triazine group, which may be attached via any possible location, such as indicated in structure (Q24). The 1,2,3-triazine is most preferred as triazine group.

In another preferred embodiment, Q comprises or is an azido group, preferably Q is an azido group. Preferably, the azide group is according to structure (Q25) as shown below.

In another preferred embodiment, Q comprises or is a nitrile oxide group, preferably Q is a nitrile oxide group. Preferably, the nitrile oxide group is according to structure (Q27) as shown below.

In another preferred embodiment, Q comprises or is a nitrone group, preferably Q is a nitrone group. Preferably, the nitrone group is according to structure (Q28) as shown below, wherein

R²⁹ is selected from the group consisting of linear or branched C₁ - C₁₂ alkyl groups and C₆ - C₁₂ aryl groups. Preferably, R²⁹ is a C₁ - C₆ alkyl group, more preferably R²⁹ is a C₁ - C₄ alkyl group. Even more preferably R²⁹ is methyl, ethyl, n-propyl, i-propyl, n-butyl, s-butyl or t-butyl. Yet even more preferably R²⁹ is methyl.

In another preferred embodiment, Q comprises or is a nitrile imine group, preferably Q is a nitrile imine group. Preferably, the nitrile imine group is according to structure (Q29) or (Q30) as shown below, wherein R³⁰ is selected from the group consisting of linear or branched C₁ - C₁₂ alkyl groups and C₆ - C₁₂ aryl groups. Preferably, R³⁰ is a C₁ - C₆ alkyl group, more preferably R³⁰ is a C₁ - C₄ alkyl group. Even more preferably R³⁰ is methyl, ethyl, n-propyl, i-propyl, n-butyl, s-butyl or t-butyl. Yet even more preferably R³⁰ is methyl.

In another preferred embodiment, Q comprises or is a diazo group, preferably Q is a diazo group. Preferably, the diazo group is according to structure (Q31) as shown below, wherein R³³ is selected from the group consisting of hydrogen or a carbonyl derivative. More preferably, R³³ is hydrogen.

In another preferred embodiment, Q comprises or is a ketone group, preferably Q is a ketone group. Preferably, the ketone group is according to structure (Q32) as shown below, wherein R³⁴ is selected from the group consisting of linear or branched C₁ - C₁₂ alkyl groups and C₆ - C₁₂aryl groups. Preferably, R³⁴ is a C₁ - C₆ alkyl group, more preferably R³⁴ is a C₁ - C₄ alkyl group. Even more preferably R³⁴ is methyl, ethyl, n-propyl, i-propyl, n-butyl, s-butyl or t-butyl. Yet even more preferably R³⁴ is methyl.

In another preferred embodiment, Q comprises or is an (O-alkyl)hydroxylamino group, preferably Q is an (O-alkyl)hydroxylamino group. Preferably, the (O-alkyl)hydroxylamino group is according to structure (Q33) as shown below.

In another preferred embodiment, Q comprises or is a hydrazine group, preferably Q is a hydrazine group. Preferably, the hydrazine group is according to structure (Q34) as shown below.

In another preferred embodiment, Q comprises or is an allenamide group, preferably Q is an allenamide group. Preferably, the allenamide group is according to structure (Q35).

In another preferred embodiment, Q comprises or is an phosphonamidate group, preferably Q is an phosphonamidate group. Preferably, the phosphonamidate group is according to structure (Q36).

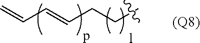

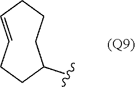

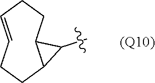

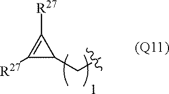

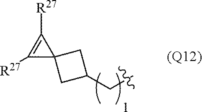

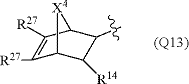

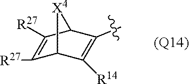

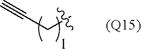

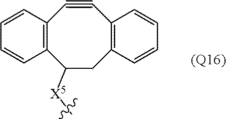

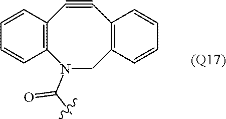

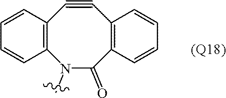

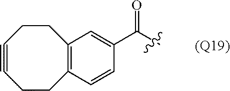

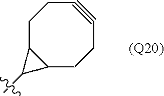

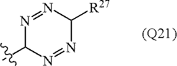

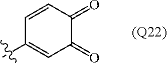

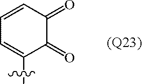

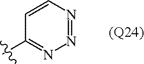

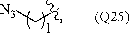

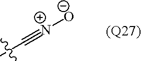

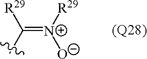

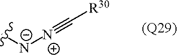

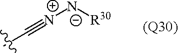

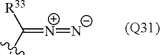

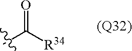

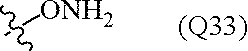

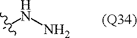

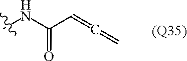

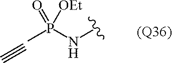

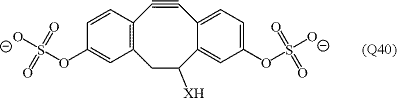

Herein, the aromatic rings in (Q16) are optionally O-sulfonylated at one or more positions, whereas the rings of (Q17) and (Q18) may be halogenated at one or more positions.

In case Q is a (hetero)cycloalkynyl group, it is preferred to Q is selected from the group consisting of (Q42) - (Q60):

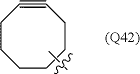

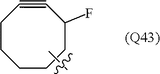

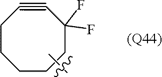

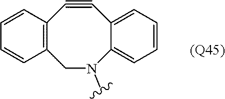

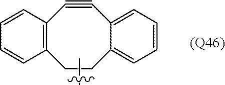

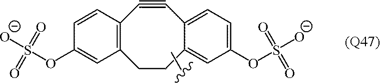

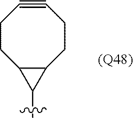

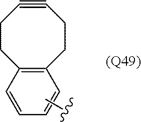

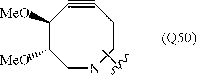

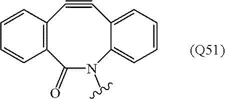

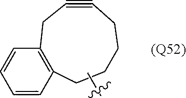

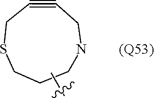

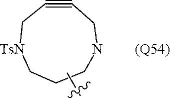

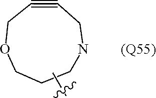

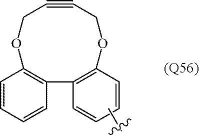

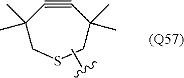

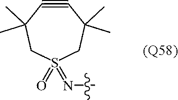

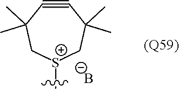

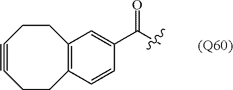

Herein, the connection to the remainder of the molecule, depicted with the wavy bond, may be to any available carbon or nitrogen atom of Q. The nitrogen atom of (Q50), (Q53), (Q54) and (Q55) may bear the connection, or may contain a hydrogen atom or be optionally functionalized. B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf. In the conjugation reaction, B⁽⁻⁾ does not need to be a pharmaceutically acceptable anion, since B⁽⁻⁾ will exchange with the anions present in the reaction mixture anyway. In case (Q59) is used for Q, the negatively charged counter-ion is preferably pharmaceutically acceptable upon isolation of the antibody-conjugate according to the invention, such that the antibody-conjugate is readily useable as medicament.

Some representative examples of reaction between F and Q and their corresponding products (connecting group Z) are depicted in FIG. 1.

The conjugation is achieved by cycloaddition. In a preferred embodiment, the conjugation is achieved by [4+2] cycloaddition or a 1,3-dipolar cycloaddition and the nucleophilic reaction is a Michael addition or a nucleophilic substitution. Thus, in a preferred embodiment of the conjugation process according to the invention, conjugation is accomplished via a [4+2] cycloaddition or a 1,3-dipolar cycloaddition, preferably the 1,3-dipolar cycloaddition.

A typical [4+2] cycloaddition is the Diels-Alder reaction, wherein Q is a diene or a dienophile. As appreciated by the skilled person, the term “diene” in the context of the Diels-Alder reaction refers to 1,3-(hetero)dienes, and includes conjugated dienes (R₂C═CR—CR═CR₂), imines (e.g. R₂C═CR—N═CR₂ or R₂C═CR—CR═NR, R₂C═N—N═CR₂) and carbonyls (e.g. R₂C═CR—CR═O or O═CR—CR═O). Hetero-Diels-Alder reactions with N— and O-containing dienes are known in the art. Any diene known in the art to be suitable for [4+2] cycloadditions may be used as reactive group Q. Preferred dienes include tetrazines as described above, 1,2-quinones as described above and triazines as described above. Although any dienophile known in the art to be suitable for [4+2] cycloadditions may be used as reactive group Q, the dienophile is preferably an alkene or alkyne group as described above, most preferably an alkyne group. For conjugation via a [4+2] cycloaddition, it is preferred that Q is a dienophile (and F is a diene), more preferably Q is or comprises an alkynyl group.

For a 1,3-dipolar cycloaddition, Q is a 1,3-dipole or a dipolarophile. Any 1,3-dipole known in the art to be suitable for 1,3-dipolar cycloadditions may be used as reactive group Q. Preferred 1,3-dipoles include azido groups, nitrone groups, nitrile oxide groups, nitrile imine groups and diazo groups. Although any dipolarophile known in the art to be suitable for 1,3-dipolar cycloadditions may be used as reactive groups Q, the dipolarophile is preferably an alkene or alkyne group, most preferably an alkyne group. For conjugation via a 1,3-dipolar cycloaddition, it is preferred that Q is a dipolarophile (and F is a 1,3-dipole), more preferably Q is or comprises an alkynyl group.

Thus, in a preferred embodiment, Q is selected from dipolarophiles and dienophiles. Preferably, Q is an alkene or an alkyne group. In an especially preferred embodiment, Q comprises an alkyne group, preferably selected from the alkynyl group as described above, the cycloalkenyl group as described above, the (hetero)cycloalkynyl group as described above and a bicyclo[6.1.0]non-4-yn-9-yl] group. More preferably Q comprises a terminal alkyne or a cyclooctyne moiety, preferably bicyclononyne (BCN), azadibenzocyclooctyne (DIBAC/DBCO) or dibenzocyclooctyne (DIBO), more preferably BCN or DIBAC/DBCO, most preferably BCN. In alternative preferred embodiment, Q is selected from the formulae (Q5), (Q6), (Q7), (Q8), (Q20) and (Q9), more preferably selected from the formulae (Q6), (Q7), (Q8), (Q20) and (Q9). Most preferably, Q is a bicyclo[6.1.0]non-4-yn-9-yl] group, preferably of formula (Q20). These groups are known to be highly effective in the conjugation with azido-functionalized antibodies.

In an especially preferred embodiment, reactive group Q comprises an alkynyl group and is according to structure (Q36):

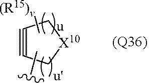

Herein:
R¹⁵ is independently selected from the group consisting of hydrogen, halogen, -OR¹⁶, —NO₂, —CN, —S(O)₂R¹⁶, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an annelated cycloalkyl or an annelated (hetero)arene substituent, and wherein R¹⁶ is independently selected from the group consisting of hydrogen, halogen, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups;
X¹⁰ is C(R¹⁷)₂, O, S or NR¹⁷, wherein R¹⁷ is R¹⁵;
u is 0, 1, 2, 3, 4 or 5;
u′ is 0, 1, 2, 3, 4 or 5;
wherein u + u′ = 5;
v = 9 or 10.

Preferred embodiments of the reactive group according to structure (Q36) are reactive groups according to structure (Q37), (Q6), (Q7), (Q8), (Q9) and (Q20).

In an especially preferred embodiment, reactive group Q comprises an alkynyl group and is according to structure (Q37):

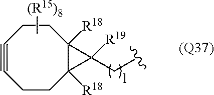

Herein:
R¹⁵ is independently selected from the group consisting of hydrogen, halogen, -OR¹⁶, —NO₂, —CN, —S(O)₂R¹⁶, C₁ - C₂₄ alkyl groups, C₅ - C₂₄ (hetero)aryl groups, C₇ - C₂₄ alkyl(hetero)aryl groups and C₇ - C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an annelated cycloalkyl or an annelated (hetero)arene substituent, and wherein R¹⁶ is independently selected from the group consisting of hydrogen, halogen, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups;
R¹⁸ is independently selected from the group consisting of hydrogen, halogen, C₁ - C₂₄30 alkyl groups, C₆ - C₂₄ (hetero)aryl groups, C₇ - C₂₄ alkyl(hetero)aryl groups and C₇ - C₂₄ (hetero)arylalkyl groups;
R¹⁹ is selected from the group consisting of hydrogen, halogen, C₁ - C₂₄ alkyl groups, C₆ - C₂₄ (hetero)aryl groups, C₇ - C₂₄ alkyl(hetero)aryl groups and C₇ - C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
— I is an integer in the range 0 to 10.

In a preferred embodiment of the reactive group according to structure (Q37), R¹⁵ is independently selected from the group consisting of hydrogen, halogen, -OR¹⁶, C₁ - C₆ alkyl groups, C₅ - C₆ (hetero)aryl groups, wherein R¹⁶ is hydrogen or C₁ - C₆ alkyl, more preferably R¹⁵ is independently selected from the group consisting of hydrogen and C₁ - C₆ alkyl, most preferably all R¹⁵ are H. In a preferred embodiment of the reactive group according to structure (Q37), R¹⁸ is independently selected from the group consisting of hydrogen, C₁ - C₆ alkyl groups, most preferably both R¹⁸ are H. In a preferred embodiment of the reactive group according to structure (Q37), R¹⁹ is H. In a preferred embodiment of the reactive group according to structure (Q37), I is 0 or 1, more preferably I is 1. An especially preferred embodiment of the reactive group according to structure (Q37) is the reactive group according to structure (Q20).

### Compounds

In a further aspect, the invention concerns compounds of structure (2):

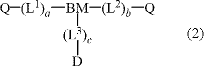

wherein:
a, b and c are each individually 0 or 1;
L¹, L² and L³ are linkers;
D is a payload;
BM is a branching moiety;
Q comprises a (hetero)cyclooctyne moiety.

Moieties a, b, c, L¹, L², L³, D, BM and Q are further defined above, which equally applies to the present aspect, including preferred embodiments defined above. In a preferred embodiment, D is a cytotoxin is further defined above. Preferred compounds of structure (2) are symmetrical, i.e. each occurrence of a/b, L¹/L² and Q is the same. Preferably, a = b = 1, more preferably also c = 1.

In the context of the present aspect, Q comprises a (hetero)cyclooctyne moiety, which is optionally substituted and may be heterocyclooctynyl group or a cyclooctynyl group, preferably a cyclooctynyl group. In a further preferred embodiment, the (hetero)cyclooctynyl group is according to structure (Q36). Preferred examples of the (hetero)cyclooctynyl group include structure (Q16), also referred to as a DIBO group, (Q17), also referred to as a DIBAC group,or (Q18), also referred to as a BARAC group, (Q19), also referred to as a COMBO group, and (Q20), also referred to as a BCN group, wherein X⁵ is O or NR²⁷, and preferred embodiments of R²⁷ are as defined above. The aromatic rings in (Q16) are optionally O-sulfonylated at one or more positions, preferably at two positions, most preferably according to (Q37), whereas the rings of (Q17) and (Q18) may be halogenated at one or more positions. A particularly preferred cyclooctynyl group is a bicyclo[6.1.0] non-4-yn-9-yl] group (BCN group), which is optionally substituted. Preferably, the bicyclo[6.1.0]non-4-yn-9-yl] group is according to structure (Q20) as shown below. In one embodiment, Q is bicyclononyne (BCN), azadibenzocyclooctyne (DIBAC/DBCO), dibenzocyclooctyne (DIBO) or sulfonylated dibenzocyclooctyne (s-DIBO), more preferably BCN or DIBAC/DBCO, most preferably BCN.

The compounds according to this aspect are ideally suitable as intermediate in the preparation of the antibody-payload conjugates according to the present invention.

### Application

The conjugates according to the invention are especially suitable in the treatment of cancer. The invention thus further concerns the use of the conjugate according to the invention in medicine. In a further aspect, the invention also concerns a method of treating a subject in need thereof, comprising administering the conjugate according to the invention to the subject. The method according to this aspect can also be worded as the conjugate according to the invention for use in treatment. The method according to this aspect can also be worded as use of the conjugate according to the invention for the manufacture of a medicament. Herein, administration typically occurs with a therapeutically effective amount of the conjugate according to the invention.

The invention further concerns a method for the treatment of a specific disease in a subject in need thereof, comprising the administration of the conjugate according to the invention as defined above. The specific disease may be selected from cancer, a viral infection, a bacterial infection, a neurological disease, an autoimmune disease, an eye disease, hypercholesterolaemia and amyloidosis, more preferable from cancer and a viral infection, most preferably the disease is cancer. The subject in need thereof is typically a cancer patient. The use of conjugate according to the invention is well-known in such treatments, especially in the field of cancer treatment, and the conjugates according to the invention are especially suited in this respect. In the method according to this aspect, the conjugate is typically administered in a therapeutically effective amount. The present aspect of the invention can also be worded as a conjugate according to the invention for use in the treatment of a specific disease in a subject in need thereof, preferably for the treatment of cancer. In other words, this aspect concerns the use of a conjugate according to the invention for the preparation of a medicament or pharmaceutical composition for use in the treatment of a specific disease in a subject in need thereof, preferably for use in the treatment of cancer.

It is preferred that the conjugate according to the invention is Fc-silent, i.e. does not significantly bind to Fc gamma receptors CD16, CD32 and CD64 when used in clinically. This is the case when G is absent, i.e. that e = 0.

Administration in the context of the present invention refers to systemic administration. Hence, in one embodiment, the methods defined herein are for systemic administration of the conjugate. In view of the specificity of the conjugates, they can be systemically administered, and yet exert their activity in or near the tissue of interest (e.g. a tumour). Systemic administration has a great advantage over local administration, as the drug may also reach tumour metastasis not detectable with imaging techniques and it may be applicable to hematological tumours.

The invention further concerns a pharmaceutical composition comprising the antibody-payload conjugate according to the invention and a pharmaceutically acceptable carrier.

### EXAMPLES

The invention is illustrated by the following examples.

### General Procedures

Chemicals were purchased from commonly used suppliers (Sigma-Aldrich, Acros, Alfa Aesar, Fluorochem, Apollo Scientific Ltd and TCI) and were used without further purification. Solvents (including dry solvents) for chemical transformations, work-up and chromatography were purchased from Aldrich (Dorset, UK) at HPLC grade, and used without further distillation. Silica gel 60 F254 analytical thin layer chromatography (TLC) plates were from Merck (Darmstadt, Germany) and visualized under UV light, with potassium permanganate stain or anisaldehyde stain. Chromatographic purifications were performed using Acros silica gel (0.06-0.200, 60A) or prepacked columns (Silicycle) in combination with a Buchi Sepacore C660 fraction collector (Flawil, Switzerland). Reversed phase HPLC purifications were performed using an Agilent 1200 system equipped with a Waters Xbridge C18 column (5 µm OBD, 30 × 100 mm, PN186002982). Deuterated solvents used for NMR spectroscopy were obtained from Cambridge Isotope Laboratories. H-Val-Ala-PABC-MMAF.TFA was obtained from Levena Biopharm, bis-mal-Lys-PEG₄-TFP ester (177) was obtained from Quanta Biodesign, O-(2-aminoethyl)-O′-(2-azidoethyl)diethylene glycol (XL07) and compounds 344 and 179 were obtained from Broadpharm, 2,3-bis(bromomethyl)-6-quinoxalinecarboxylic acid (178) was obtained from ChemScene and 32-azido-5-oxo-3,9,12,15,18,21,24,27,30-nonaoxa-6-azadotriacontanoicacid (348) was obtained from Carbosynth.

### General Procedure for Mass Spectral Analysis of Monoclonal Antibodies and ADC

Prior to mass spectral analysis, IgG was treated with IdeS (Fabricator™) for analysis of the Fc/2 fragment. A solution of 20 µg (modified) IgG was incubated for 1 hour at 37° C. with 0.5 µL IdeS (50 U/µL) in phosphate-buffered saline (PBS) pH 6.6 in a total volume of 10 µL. Samples were diluted to 40 µL followed by electrospray ionization time-of-flight (ESI-TOF) analysis on a JEOL AccuTOF. Deconvoluted spectra were obtained using Magtran software.

### General Procedure for Analytical RP-HPLC

Prior to RP-HPLC analysis, IgG was treated with IdeS, which allows analysis of the Fc/2 fragment. A solution of (modified) IgG (100 µL, 1 mg/mL in PBS pH 7.4) was incubated for 1 hour at 37° C. with 1.5 µL IdeS/Fabricator™ (50 U/µL) in phosphate-buffered saline (PBS) pH 6.6. The reaction was quenched by adding 49% acetonitrile, 49% water, 2% formic acid (100 µL). RP-HPLC analysis was performed on an Agilent 1100 series (Hewlett Packard). The sample (10 µL) was injected with 0.5 mL/min onto a ZORBAX Poroshell 300SB-C8 column (1×75 mm, 5 µm, Agilent) with a column temperature of 70° C. A linear gradient was applied in 25 minutes from 30 to 54% acetonitrile and water in 0.1% TFA.

### General Procedure for Analytical HPLC-SEC

HPLC-SEC analysis was performed on an Agilent 1100 series (Hewlett Packard). The sample (4µL, 1 mg/mL) was injected with 0.86 mL/min onto a Xbridge BEH200A (3.5 µM, 7.8x300 mm, PN186007640 Waters) column. Isocratic elution using 0.1 M sodium phosphate buffer pH 6.9 (NaH₂PO₄/Na₂HPO₄) was performed for 16 minutes.

### Example 1. Synthesis of Compound 102

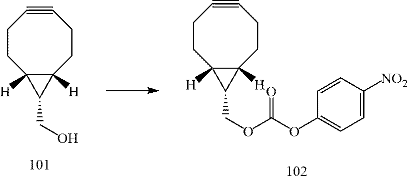

To a cooled (0° C.) solution of 4-nitrophenyl chloroformate (30.5 g, 151 mmol) in DCM (500 mL) was added pyridine (24.2 mL, 23.7 g, 299 mmol). A solution of BCN-OH (101, 18.0 g, 120 mmol) in DCM (200 mL) was added dropwise to the reaction mixture. After the addition was completed, a saturated aqueous solution of NH₄Cl (500 mL) and water (200 mL) were added. After separation, the aqueous phase was extracted with DCM (2 × 500 mL). The combined organic phases were dried (Na₂SO₄) and concentrated. The crude material was purified by silica gel chromatography and the desired product 102 was obtained as an off-white solid (18.7 g, 59 mmol, 39%). ¹H NMR (400 MHz, CDCl₃) δ (ppm) 8.32-8.23 (m, 2 H), 7.45-7.34 (m, 2 H), 4.40 (d, J = 8.3 Hz, 2 H), 2.40-2.18 (m, 6 H), 1.69- 1.54 (m, 2 H), 1.51 (quintet, J = 9.0 Hz, 1 H), 1.12-1.00 (m, 2 H)

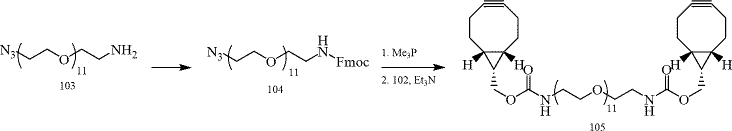

### Example 2. Synthesis of Compound 104

To a cooled solution (-5° C.) of azido-PEG₁₁-amine (103) (182 mg, 0.319 mmol) in THF (3 mL) were added a 10% aqueous NaHCO₃ solution (1.5 mL) and 9-fluorenylmethoxycarbonyl chloride (99 mg, 0.34 mmol) dissolved in THF (2 mL). After 2 h, EtOAc (20 mL) was added and the mixture was washed with brine (2 × 6 mL), dried over MgSO₄, and concentrated. Purification by silica gel column chromatography (0 → 11% MeOH in DCM) gave 104 as a clear oil in 98% yield (251 mg, 0.316 mmol). LCMS (ESI+) calculated for C₃₉H₆₀N₄O₁₃⁺(M+Na⁺) 815.42 found 815.53.

### Example 3. Synthesis of Compound 105

A solution of 104 (48 mg, 0.060 mmol) in THF (3 mL) and water (0.2 mL) was prepared and cooled down to 0° C. Trimethylphosphine (1 M in toluene, 0.24 mL, 0.24 mmol) was added and the mixture was left stirring for 23 h. The water was removed via extraction with DCM (6 mL). To this solution, (1R,8S,9 s)-bicyclo[6.1.0]non-4-yn-9-ylmethyl (4-nitrophenyl) carbonate (102) (25 mg, 0.079 mmol) and triethylamine (10 µL, 0.070 mmol) were added. After 27 h, the mixture was concentrated and the residue was dissolved in DMF (3 mL), followed by the addition of piperidine (400 µL). After 1 h, the mixture was concentrated and the residue was purified by silica gel column chromatography (0 → 21% MeOH in DCM), which gave 105 as a colorless oil (8.3 mg, 0.0092 mmol). LCMS (ESI+) calculated for C₄₆H₇₆N₂O₁₅+(M+NH₄+) 914.52 found 914.73.

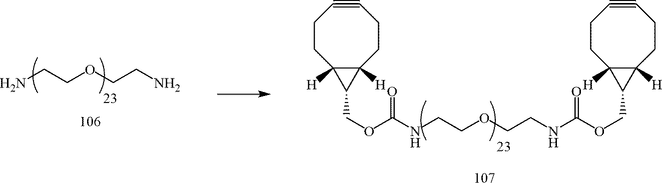

### Example 4. Synthesis of Compound 107

A solution of (1R,8S,9 s)-bicyclo[6.1.0]non-4-yn-9-ylmethyl (4-nitrophenyl) carbonate (102) (4.1 mg, 0.013 mmol) in dry DCM (500 µL) was slowly added to a solution of amino-PEG23-amine (106) (12.3 mg, 0.0114 mmol) in dry DCM (500 µL). After 20 h, the mixture was concentrated and the residue was purified by silica gel column chromatography (0 → 25% MeOH in DCM) which gave the desired compound 107 in 73% yield (12 mg, 0.0080 mmol). LCMS (ESI+) calculated for C₇₀H₁₂₄N₂O₂₇+(M+ NH₄+) 1443.73 found 1444.08.

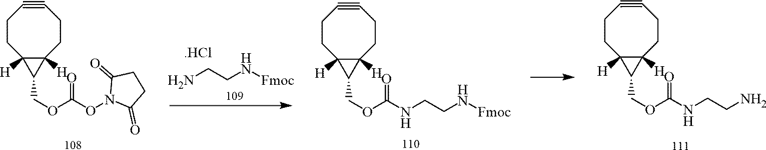

### Example 5. Synthesis of Compound 108

To a solution of BCN-OH (101, 21.0 g, 0.14 mol) in MeCN (450 mL) were added disuccinimidyl carbonate (53.8 g, 0.21 mol) and triethylamine (58.5 mL, 0.42 mol). After the mixture was stirred for 140 minutes, it was concentrated in vacuo and the residue was co-evaporated once with MeCN (400 mL). The residue was dissolved in EtOAc (600 mL) and washed with H₂O (3 × 200 mL). The organic layer was dried over Na₂SO₄ and concentrated in vacuo. The residue was purified by silica gel column chromatography (0 → 4% EtOAc in DCM) and gave 108 (11.2 g, 38.4 mmol, 27% yield) as a white solid. ¹H NMR (400 MHz, CDCl₃): δ (ppm) 4.45 (d, 2H, J = 8.4 Hz), 2.85 (s, 4 H), 2.38-2.18 (m, 6 H), 1.65- 1.44 (m, 3 H), 1.12-1.00 (m, 2 H).

### Example 6. Synthesis of Compound 110

To a solution of (1RX,8S,9 s)-bicyclo[6.1.0]non-4-yn-9-ylmethyl N-succinimidyl carbonate (108) (500 mg, 1.71 mmol) in DCM (15 mL) were added triethylamine (718 uL, 5.14 mmol) and mono-Fmoc ethylenediamine hydrochloride (109) (657 mg, 2.06 mmol). The mixture was stirred for 45 min,diluted with EtOAc (150 mL) and washed with a 50% saturated aqueous NH₄Cl solution (50 mL). The aqueous layer was extracted with EtOAc (50 mL) and the combined organic layers were washed with H₂O (10 mL). The combined organic extracts were concentrated in vacuo and the half of the residue was purified by silica gel column chromatography (0 → 3% MeOH in DCM) which gave the desired compound 110 in 42% yield (332 mg, 0.72 mmol). ¹H NMR (400 MHz, CDCl₃) δ (ppm) 7.77 (d, J = 7.5 Hz, 2 H), 7.59 (d, J = 7.4 Hz, 2 H), 7.44-7.37 (m, 2 H), 7.36-7.28 (m, 2 H), 5.12 (br s, 1 H), 4.97 (br s, 1 H), 44.41 (d, J = 6.8 Hz, 2 H), 4.21 (t, J = 6.7 Hz, 1 H), 4.13 (d, J = 8.0 Hz, 2 H), 3.33 (br s, 4 H), 2.36-2.09 (m, 6 H), 1.67-1.45 (m, 2 H), 1.33 (quintet, J = 8.6 Hz, 1 H), 1.01-0.85 (m, 2H). LCMS (ESI+) calculated for C₂₈H₃₁N₂O₄+(M+ H⁺) 459.23 found 459.52.

### Example 7. Synthesis of Compound 111

Compound 110 (327 mg, 0.713 mmol) was dissolved in DMF (6 mL) and piperidine (0.5 mL) was added. After 2 h, the mixture was concentrated and the residue was purified by silica gel column chromatography (0 → 32% 0.7 N NH₃ MeOH in DCM), which gave the desired compound 111 as a yellow oil (128 mg, 0.542 mmol, 76%). ¹H-NMR (400 MHz, CDCl₃) δ (ppm, rotamers) 5.2 (bs, 1 H), 4.15 (d, J = 8.0 Hz, 2 H), 3.48-3.40 (m, ⅔H), 3.33-3.27 (m, ⅔H), 3.27-3.19 (m, 1 ⅓H), 2.85-2.80 (m, 1 ⅓H), 2.36-2.17 (m, 6 H), 1.67-1.50 (m, 2 H), 1.36 (quintet, J = 8.5 Hz, 1 H), 1.01-0.89 (m, 2 H).

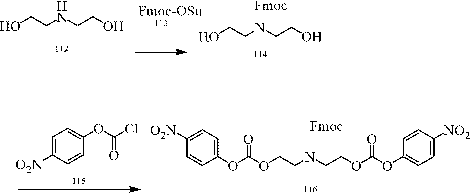

### Example 8. Synthesis of Compound 114

To a solution of diethanolamine (112) (208 mg, 1.98 mmol) in water (20 mL) were added MeCN (20 mL), NaHCO₃ (250 mg, 2.97 mmol) and a solution of Fmoc-OSu (113) (601 mg, 1.78 mmol) in MeCN (20 mL). The mixture was stirred for 2 h and DCM (50 mL) was added. After separation, the organic phase was washed with water (20 mL), dried (Na₂SO₄) and concentrated. The desired product 114 was obtained as a colorless thick oil (573 mg, 1.75 mmol, 98%). ¹H NMR (400 MHz, CDCl₃) δ (ppm) 7.79-7.74 (m, 2 H), 7.60-7.54 (m, 2 H), 7.44-7.37 (m, 2 H), 7.36-7.30 (m, 2 H), 4.58 (d, J = 5.4 Hz, 2 H), 4.23 (t, J = 5.3 Hz, 1 H), 3.82-3.72 (m, 2 H), 3.48-3.33 (m, 4 H), 3.25-3.11 (m, 2 H).

### Example 9. Synthesis of Compound 116

To a solution of 114 (567 mg, 1.73 mmol) in DCM (50 mL) were added 4-nitrophenyl chloroformate (115) (768 mg, 3.81 mmol) and Et₃N (1.2 mL, 875 mg). The mixture was stirred for 18 h and concentrated. The residue was purified by silica gel chromatography (0% → 10% MeOH in DCM, then 20% → 70% EtOAc in heptane, which afforded 32 mg (49 µmol, 2.8%) of the desired product 116. ¹H NMR (400 MHz, CDCl₃) δ (ppm) 8.31-8.20 (m, 4 H), 7.80-7.74 (m, 2 H), 7.59-7.54 (m, 2 H), 7.44-7.37 (m, 2 H), 7.37-7.29 (m, 6 H), 4.61 (d, J = 5.4 Hz, 2 H), 4.39 (t, J = 5.1 Hz, 2 H), 4.25 (t, J = 5.5 Hz, 1 H), 4.02 (t, J = 5.0 Hz, 2 H), 3.67 (t, J = 4.8 Hz, 2 H), 3.45 (t, J = 5.2 Hz, 2 H).

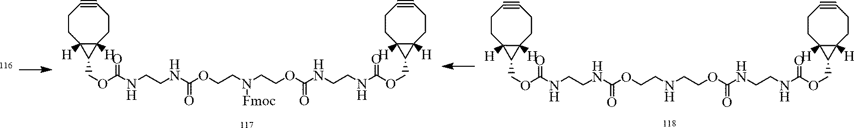

### Example 10. Synthesis of Compound 117

To a solution of 116 (34 mg, 0.050 mmol) in DCM (2 mL) were added 111 (49 mg, 0.21 mmol) and triethylamine (20 µL, 0.14 mmol). The mixture was left stirring overnight at room temperature. After 23 h, the mixture was concentrated. Purification by silica gel column chromatography (0 → 40% MeOH in DCM) gave 117 as a white solid in 61% yield (27 mg, 0.031 mmol). LCMS (ESI+) calculated for C₄₇H₅₇N₅O₁₀+(M+H⁺) 851.41 found 852.49.

### Example 11. Synthesis of Compound 118

Compound 118 was obtained during the preparation of 117 (3.8 mg, 0.0060 mmol). LCMS (ESI+) calculated for C₃₂H₄₇N₅O₈+(M+H⁺) 629.34 found 630.54.

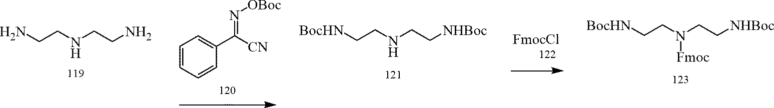

### Example 12. Synthesis of Compound 121

A solution of diethylenetriamine (119) (73 µL, 0.67 mmol) and triethylamine (283 µL, 2.03 mmol) in THF (6 mL) was cooled down to -5° C. and placed under a nitrogen atmosphere. 2-(Boc-oxyimino)-2-phenylacetonitrile (120) (334 mg, 1.35 mmol) was dissolved in THF (4 mL) and slowly added to the cooled solution. After 2.5 h, the ice bath was removed and the mixture was stirred for an additional of 2.5 h at room temperature, and concentrated in vacuo. The residue was redissolved in DCM (15 mL) and washed with a 5% aqueous NaOH solution (2 × 5 mL), brine (2 × 5 mL) and dried over MgSO₄. Purification by silica gel column chromatography (0 → 14% MeOH in DCM) gave 121 as a colorless oil in 91% yield (185 mg, 0.610 mmol). ¹H-NMR (400 MHz, CDCl₃) δ (ppm) 5.08 (s, 2 H), 3.30-3.12 (m, 4 H), 2.74 (t, J = 5.9 Hz, 4 H), 1.45 (s, 18 H).

### Example 13. Synthesis of Compound 123

To a cooled solution (-10° C.) of 121 (33.5 mg, 0.110 mmol) in THF (2 mL) were added a 10% aqueous NaHCO₃ solution (500 µL) and 9-fluorenylmethoxycarbonyl chloride (122) (34 mg, 0.13 mmol) dissolved in THF (1 mL). After 1 h, the mixture was concentrated and the residue was redissolved in EtOAc (10 mL), washed with brine (2 × 5 mL), dried over Na₂SO₄, and concentrated. Purification by silica gel column chromatography (0 → 50% MeOH in DCM) gave 123 in 86% yield (50 mg, 0.090 mmol). ¹H-NMR (400 MHz, CDCl₃) δ (ppm) 7.77 (d, J = 7.4 Hz, 2 H), 7.57 (d, J = 7.4 Hz, 2 H), 7.43-7.38 (m, 2 H), 7.36-7.31 (m, 2 H), 5.57 (d, J = 5.2 Hz, 2 H), 4.23 (t, J = 5.1 Hz, 1 H), 3.40-2.83 (m, 8 H), 1.41 (s, 18 H).

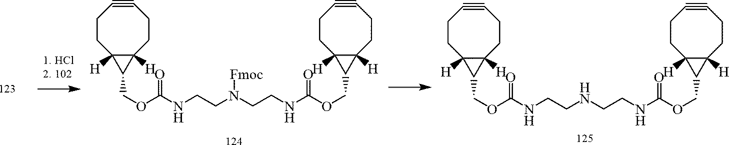

### Example 14. Synthesis of Compound 124

To a solution of 123 (50 mg, 0.095 mmol) in DCM (3 mL) was added 4 M HCl in dioxane (200 µL). The mixture was stirred for 19 h, concentrated and a white solid was obtained (35 mg). without purification, the deprotected intermediate and (1R,8S,9s)-bicyclo[6.1.0]non-4-yn-9-ylmethyl (4-nitrophenyl) carbonate (102) (70 mg, 0.22 mmol) were dissolved in DMF (3 mL) and triethylamine (34 µL, 0.24 mmol) was added. After 2 h, the mixture was concentrated and the residue was purified by silica gel column chromatography (0 → 25% MeOH in DCM) to yield 124 in 48% (31 mg, 0.045 mmol). LCMS (ESI+) calculated for C₄₁H₄₇N₂O₆+ (M+H⁺) 677.35 found 678.57.

### Example 15. Synthesis of Compound 125

To a solution of 124 (10 mg, 0.014 mmol) in DMF (500 µL) was added piperidine (20 µL). After 3.5 h, the mixture was concentrated. Purification by silica gel column chromatography (0 → 20% MeOH in DCM) gave 125 in 58% yield (3.7 mg, 0.0080 mmol). LCMS (ESI+) calculated for C₂₆H₃₇N₃O₄+ (M+H⁺) 455.28 found 456.41.

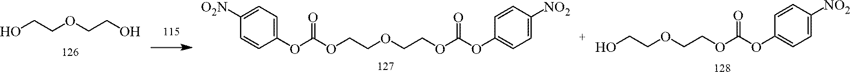

### Example 16. Synthesis of Compound 127 and 128

To a solution of diethyleneglycol (126) (446 µL, 0.50 g, 4.71 mmol) in DCM (20 mL) were added 4-nitrophenol chloroformate (115) (1.4 g, 7.07 mmol) and Et₃N (3.3 \.mL, 2.4 g, 23.6 mmol). The mixture was stirred, filtered and concentrated in vacuo (at 55° C.). The residue was purified by silica gel chromatography (15% → 75% EtOAc in heptane) and two products were isolated. Product 127 was obtained as a white solid (511 mg, 1.17 mmol, 25%).¹H NMR (400 MHz, CDCl₃) δ (ppm) 8.31-8.23 (m, 4 H), 7.43-7.34 (m, 4 H), 4.54-4.44 (m, 4 H), 3.91-3.83 (m, 4 H). Product 128 was obtained as a colorless oil (321 mg, 1.18 mmol, 25%).¹H NMR (400 MHz, CDCl₃) δ (ppm) 8.32-8.24 (m, 2 H), 7.43-7.36 (m, 2 H), 4.50-4.44 (m, 2 H), 3.86-3.80 (m, 2 H), 3.81-3.74 (m, 2 H), 3.69-3.64 (m, 2 H).

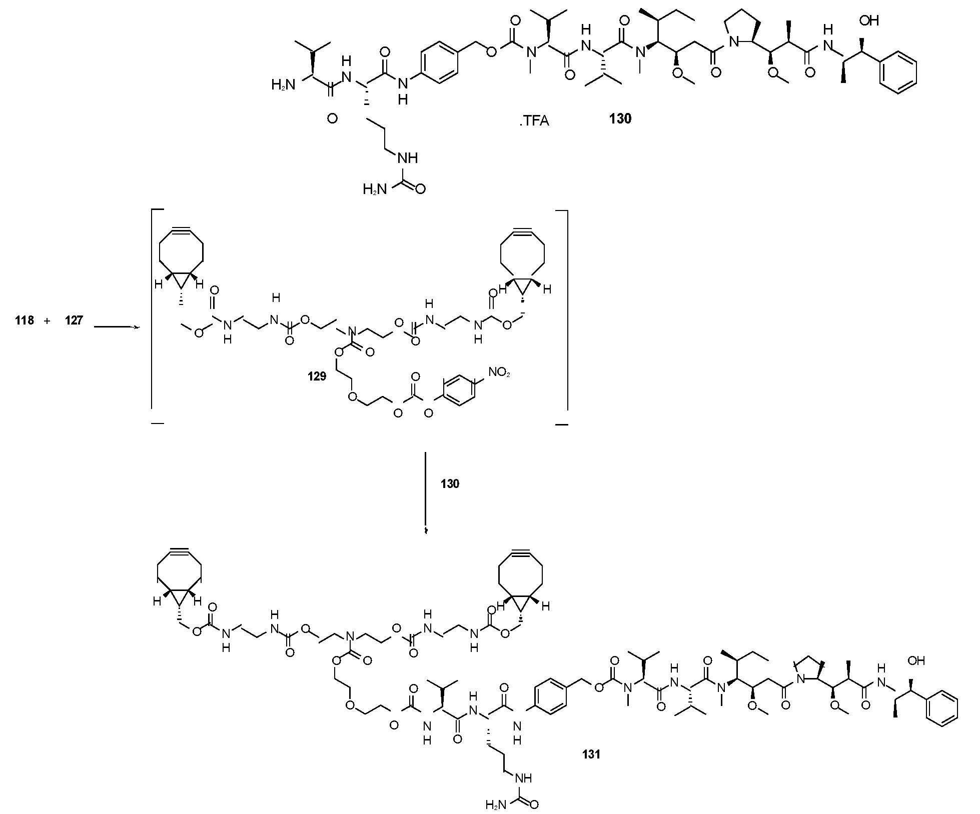

### Example 17. Synthesis of Compound 131

To a solution of 118 (2.3 mg, 3.7 µmol) in DMF (295 µL) was added a solution of 127 (3.2 mg, 7.4 µmol) in DMF (65 µL) and Et₃N (1.6 µL, 1.1 mg, 11.1 µmol). The mixture was left standing for 17 h and a solution of HOBt (0.5 mg, 3.7 umol) in DMF (14 µL) was added. After 4 h, Et₃N (5.2 µL, 3.8 mg, 37 µmol) and a solution of vc-PABC-MMAE.TFA (130, 13.8 mg, 11 µmol) in DMF (276 µL) were added. After 3 d, the mixture was purified by RP HPLC (C18, 30% → 90% MeCN (1% AcOH) in water (1% AcOH). The desired product 131 was obtained as a colorless film (1.5 mg, 0.78 µmol, 21%). LCMS (ESI+) calculated for C₉₆H₁₄₈N₁₅O₂₅+ (M+H⁺) 1911.08 found 1912.08.

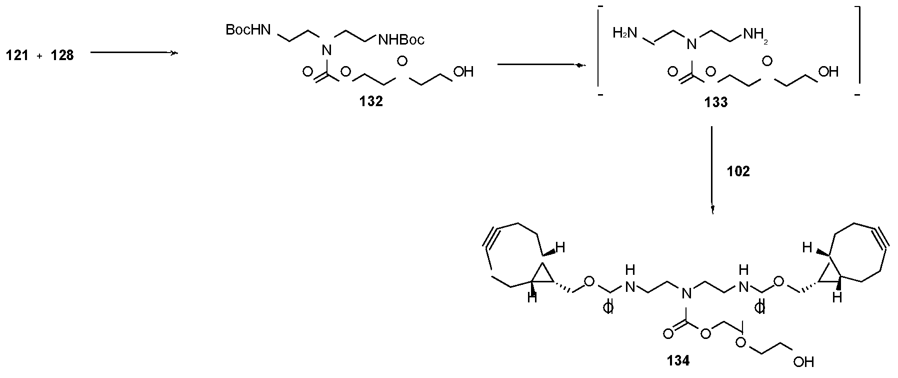

### Example 18. Synthesis of Compound 132

To a solution of 121 (168 mg, 0.554 mmol) in DCM (2 mL), were added a solution of 128 (240 mg, 0.89 mmol) in DCM (1 mL), DCM (1 mL) and Et₃N (169 mg, 233 µL). The mixture was stirred for 17 h, concentrated and purified by silica gel chromatography (gradient of EtOAc in heptane). The desired product 132 was obtained as a slightly yellow oil (85 mg, 0.20 mmol, 35%). ¹H NMR (400 MHz, CDCl₃) δ (ppm) 5.24-5.02 (m, 2 H), 4.36-4.20 (m, 3 H), 3.84-3.67 (m, 4 H), 3.65-3.58 (m, 2 H), 3.47-3.34 (m, 4 H), 3.34-3.18 (m, 4 H), 1.44 (bs, 18 H).

### Example 19. Synthesis of Compound 134

To a solution of 132 (81 mg, 0.19 mmol) in DCM (3 mL) was added 4 N HCI in dioxane (700 µL). The mixture was stirred for 19 h, concentrated and the residue was taken up in DMF (0.5 mL). Et₃N (132 µL, 96 mg, 0.95 mmol), DMF (0.5 mL) and (1R,8S,9s)-bicyclo[6.1.0]non-4-yn-9-ylmethyl (4-nitrophenyl) carbonate (102) (132 mg, 0.42 mmol) were added and the resulting mixture was stirred for 2 h. The mixture was concentrated and the residue was purified by silica gel chromatography (0% → 3% MeOH in DCM). The desired product 134 was obtained as a colorless film (64 mg, 0.11 mmol, 57%). ¹H NMR (400 MHz, CDCl₃) δ (ppm) 4.31-4.23 (m, 2 H), 4.22-4.08 (m, 4 H), 3.80-3.68 (m, 4 H), 3.66-3.58 (m, 2 H), 3.50-3.28 (m, 8 H), 2.80-2.65 (m, 1 H), 2.40-2.10 (m, 12 H), 1.68-1.48 (m, 4 H), 1.35 (quintet, J = 8.1 Hz, 1 H), 1.02-0.87 (m, 2 H). LCMS (ESI+) calculated for C₃₁H₄₆N3O₈⁺ (M+H⁺) 588.33 found 588.43.

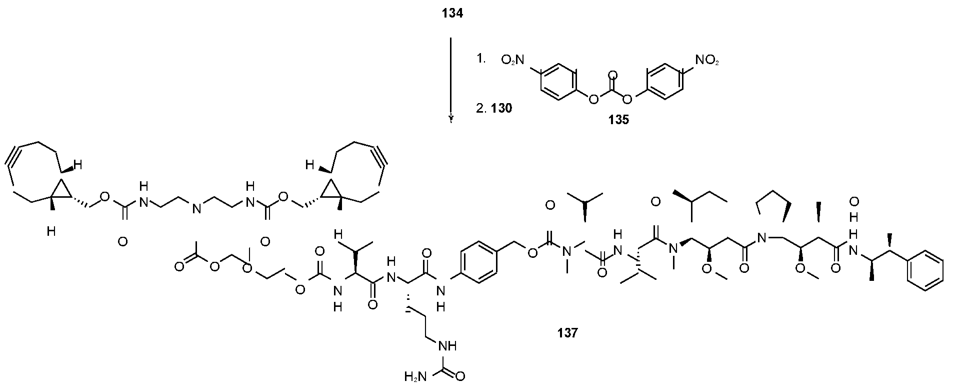

### Example 20. Synthesis of Compound 137

To a solution of 134 (63 mg, 0.11 mmol) in DCM (1 mL) was added bis(4-nitrophenyl) carbonate (35) (32.6 mg, 0.107 mmol) and Et₃N (32.5 mg, 45 µL, 0.32 mmol). After 2 h, 77 µL was removed from the main reaction mixture, a solution of vc-PABC-MMAE.TFA (130, 10 mg, 8.1 µmol) in DMF (200 µL) and Et₃N (3.4 µL, 2.5 mg, 24 µmol) were added. After 18 h, 2.2′-(ethylenedioxy)bis(ethylamine) (4.9 µL, 5.0 mg, 34 µmol) was added and the mixture was left standing for 45 min. The mixture was purified by RP HPLC (C18, 30% → 90% MeCN (1% AcOH) in water (1% AcOH). The desired product 137 was obtained as a colorless film (8.7 mg, 5.0 µmol, 61%). LCMS (ESI+) calculated for C₉₀H₁₃₈N₁₃O₂₁⁺(M+H⁺) 1737.01 found 1738.01.

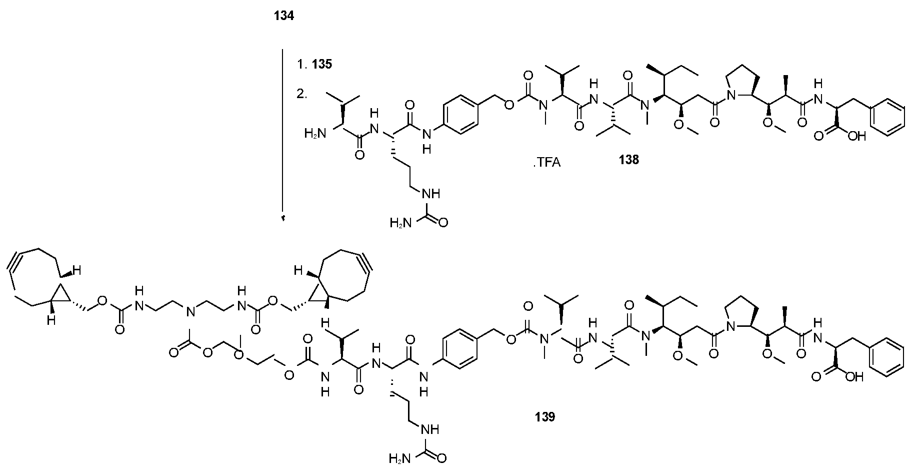

### Example 21. Synthesis of Compound 139

To a solution of 134 (63 mg, 0.11 mmol) in DCM (1 mL) was added bis(4-nitrophenyl) carbonate (35) (32.6 mg, 0.107 mmol) and Et₃N (32.5 mg, 45 µL, 0.32 mmol). After 20 h, 77 µL was removed from the main reaction mixture, a solution of vc-PABC-MMAF.TFA (138, 9.6 mg, 8.2 µmol) in DMF (240 µL) and Et₃N (3.4 µL, 2.5 mg, 24 µmol) were added. After 3 h, 2,2′-(ethylenedioxy)bis(ethylamine) (20 µL, 20 mg, 0.14 mmol) was added and the mixture was left standing for 20 min. The mixture was purified by RP HPLC (C18, 30% → 90% MeCN (1% AcOH) in water (1% AcOH). The desired product 139 was obtained as a colorless film (5.3 mg, 3.2 µmol, 39%). LCMS (ESI+) calculated forC₈₇H₁₃₀N11O₂₁⁺ (M+H) 1664.94 found 1665.99.

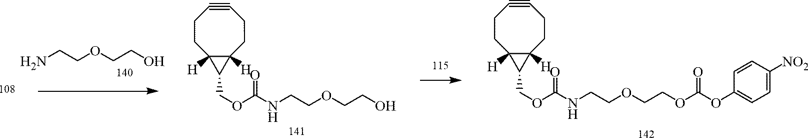

### Example 22. Synthesis of Compound 141

To a solution of (1R,8S,9S)-bicyclo[6.1.0]non-4-yn-9-ylmethyl N-succinimidyl carbonate (108) (16.35 g, 56.13 mmol) in DCM (400 ml) were added 2-(2-aminoethoxy)ethanol (140) (6.76 ml, 67.35 mmol) and triethylamine (23.47 ml, 168.39 mmol). The resulting pale yellow solution was stirred at rt for 90 min. The mixture was concentrated in vacuo and the residue was co-evaporated once with acetonitrile (400 mL). The resulting oil was dissolved in EtOAc (400 mL) and washed with H₂O (3 × 200 mL). The organic layer was concentrated in vacuo. The residue was purified by silica gel column chromatography (50% → 88% EtOAc in heptane) and gave 141 (11.2 g, 39.81 mmol, 71% yield) as a pale yellow oil. ¹H-NMR (400 MHz, CDCl₃): δ (ppm) 5.01 (br s, 1 H), 4.17 (d, 2 H, J = 12.0 Hz), 3.79-3.68 (m, 2 H), 3.64-3.50 (m, 4 H), 3.47-3.30 (m, 2 H), 2.36-2.14 (m, 6 H), 1.93 (br s, 1 H), 1.68-1.49 (m, 2 H), 1.37 (quintet, 1 H, J = 8.0 Hz), 1.01-0.89 (m, 2 H).

### Example 23. Synthesis of Compound 142

To a solution of 141 (663 mg, 2.36 mmol) in DCM (15 mL) were added triethylamine (986 uL, 7.07 mmol) and 4-nitrophenyl chloroformate (115) (712 mg, 3.53 mmol). The mixture was stirred for 4 h and concentrated in vacuo. Purification by silica gel column chromatography (0 → 20% EtOAc in heptane) gave 142 (400 mg, 0.9 mmol, yield 38%) as a pale yellow oil. H-NMR (400 MHz, CDCl₃) δ (ppm) 8.29 (d, J = 9.4 Hz, 2 H), 7.40 (d, J = 9.3 Hz, 2 H), 5.05 (br s, 1 H), 4.48-4.41 (m, 2 H), 4.16 (d, J = 8.0 Hz, 2 H), 3.81-3.75 (m, 2 H), 3.61 (t, J = 5.0 Hz, 2H), 3.42 (q, J = 5.4 Hz, 2H), 2.35-2.16 (m, 6H), 1.66-1.50 (m, 2H), 1.35 (quintet, J = 8.6 Hz, 1H), 1.02-0.88 (m, 2H). LCMS (ESI+) calculated for C₂₂H₂₆N₂NaO₈⁺(M+Na⁺) 469.16 found 469.36.

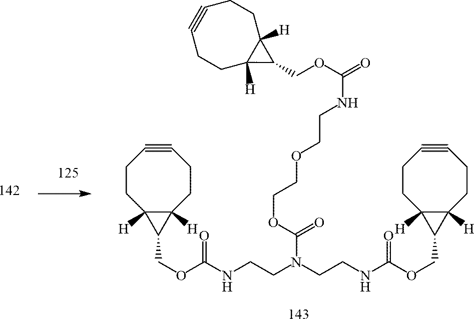

### Example 24. Synthesis of Compound 143

A solution of 142 (2.7 mg, 6.0 µmol) in DMF (48 µL) and Et₃N (2.1 µL, 1.5 mg, 15 µmol) were added to a solution of 125 (2.3 mg, 5.0 µmol) in DMF (0.32 mL). The mixture was left standing for 4 d, diluted with DMF (100 µL) and purified by RP HPLC (C18, 30% → 100% MeCN (1% AcOH) in water (1% AcOH). The product 143 was obtained as a colorless film (2.8 mg, 3.7 µmol, 74%). LCMS (ESI+) calculated for C₄₂H₅₉N₄O₉⁺ (M+H) 763.43 found 763.53.

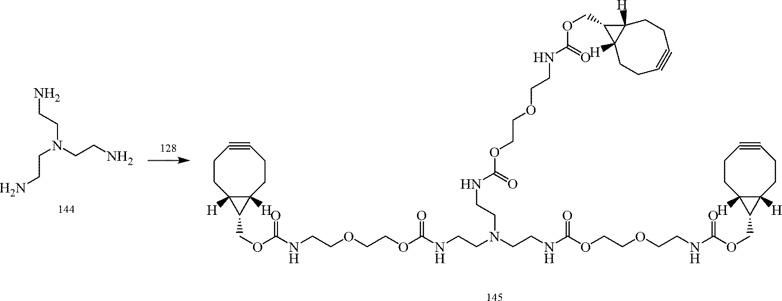

### Example 25. Synthesis of Compound 145

To a solution of 128 (200 mg, 0.45 mmol) in DCM (1 mL) were added triethylamine (41.6 uL, 0.30 mmol) and tris(2-aminoethyl)amine 144 (14.9 uL, 0.10 mmol). After stirring the mixture for 150 minutes, it was concentrated in vacuo. The residue was purified by silica gel column chromatography (25% → 100% EtOAc in DCM then 0% → 10% MeOH in DCM) and gave 145 in 43% yield (45.4 mg, 42.5 umol) as a yellow oil. ¹H NMR (400 MHz, CDCl₃): δ (ppm) 5.68-5.18 (m, 6H), 4.32-4.18 (m, 6H), 4.18-4.11 (d, J = 7.9 Hz, 6H), 3.74-3.61 (m, 6H), 3.61-3.51 (m, 6H), 3.43-3.29 (m, 6H), 3.29-3.15 (m, 6H), 2.65-2.47 (m, 6H), 2.37-2.16 (m, 18H), 1.69-1.49 (m, 6H), 1.35 (quintet, J = 8.9 Hz, 3H), 1.03-0.87 (m, 6H).

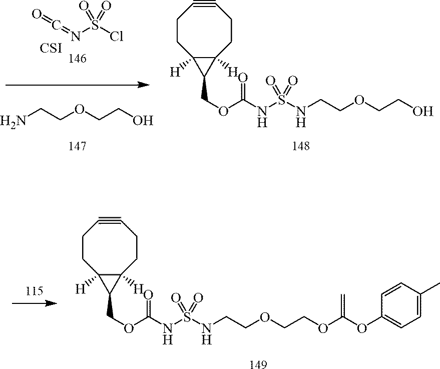

### Example 26. Synthesis of Compound 148

To a solution of BCN-OH (101) (3.0 g, 20 mmol) in DCM (300 mL) was added CSI (146) (1.74 mL, 2.83 g, 20 mmol). After the mixture was stirred for 15 min, Et₃N (5.6 mL, 4.0 g, 40 mmol) was added. The mixture was stirred for 5 min and 2-(2-aminoethoxy)ethanol (147) (2.2 mL, 2.3 g, 22 mmol) was added. The resulting mixture was stirred for 15 min and saturated aqueous NH₄Cl (300 mL) was added. The layers were separated, and the aqueous phase was extracted with DCM (200 mL). The combined organic layers were dried (Na₂SO₄) and concentrated. The residue was purified by silica gel chromatography (0% to 10% MeOH in DCM). The fractions, containing the desired product, were concentrated. The residue was taken up in EtOAc (100 mL) and concentrated. The desired product 148 was obtained as a slightly yellow oil (4.24 g, 11.8 mmol, 59%). ¹H NMR (400 MHz, CDCl₃) δ (ppm) 5.99-5.79 (bs, 1H), 4.29 (d, J = 8.3 Hz, 2H), 3.78-3.74 (m, 2H), 3.66-3.56 (m, 4H), 3.37-3.30 (m, 2H), 2.36-2.16 (m, 6H), 1.63-1.49 (m, 2H), 1.40 (quintet, J = 8.7 Hz, 1H), 1.05-0.94 (m, 2H).

### Example 27. Synthesis of Compound 149

To a solution of 148 (3.62 g, 10.0 mmol) in DCM (200 mL) were added 4-nitrophenyl chloroformate (15) (2.02 g, 10.0 mmol) and Et₃N (4.2 mL, 3.04 g, 30.0 mmol). The mixture was stirred for 1.5 h and concentrated. The residue was purified by silica gel chromatography (20% → 70% EtOAc (1% AcOH) in heptane (1% AcOH). The product 149 was obtained as a white foam (4.07 g, 7.74 mmol, 74%). ¹H NMR (400 MHz, CDCl₃) δ (ppm) 8.32-8.26 (m, 2H), 7.45-7.40 (m, 2H), 5.62-5.52 (m, 1H), 4.48-4.42 (m, 2H), 4.28 (d, J = 8.2 Hz, 2H), 3.81-3.76 (m, 2H), 3.70-3.65 (m, 2H), 3.38-3.30 (m, 2H), 2.35-2.16 (m, 6H), 1.62-1.46 (m, 2H), 1.38 (quintet, J = 8.7 Hz, 1H), 1.04-0.93 (m, 2H).

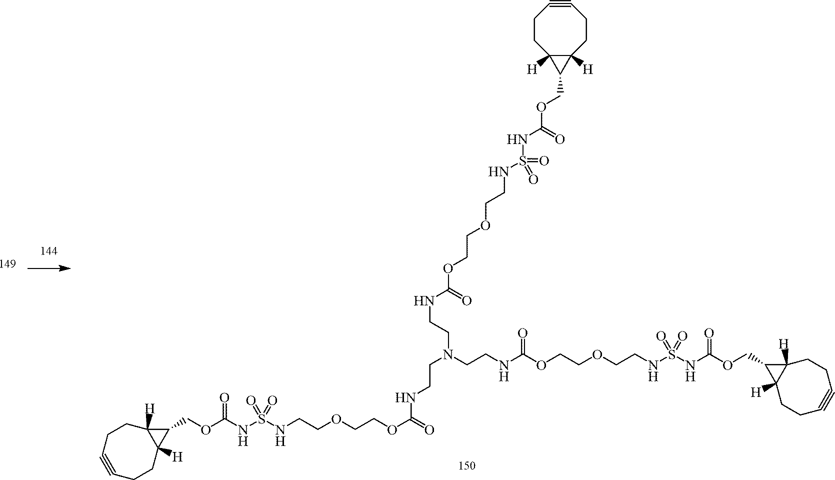

### Example 28. Synthesis of Compound 150

To a solution of 149 (200 mg, 0.38 mmol) in DCM (1 mL) were added triethylamine (35.4 uL, 0.24 mmol) and tris(2-aminoethyl)amine (144) (12.6 uL, 84.6 umol). The mixture was stirred for 120 min and concentrated in vacuo. The residue was purified by silica gel column chromatography (25% → 100% EtOAc in DCM then 0% → 10% MeOH in DCM) and gave 150 in 36% yield (40.0 mg, 30.6 umol) as a white foam. ¹H NMR (400 MHz, CDCl₃): δ (ppm) 6.34-5.72 (m, 6H), 4.34-4.18 (m, 12H), 3.76-3.58 (m, 12H), 3.43-3.30 (m, 6H), 3.30-3.18 (m, 6H), 2.64-2.49 (m, 6H), 2.38-2.14 (m, 18H), 1.65-1.47 (m, 6H), 1.39 (quintet, J = 9.1 Hz, 3H), 1.06-0.90 (m, 6H).

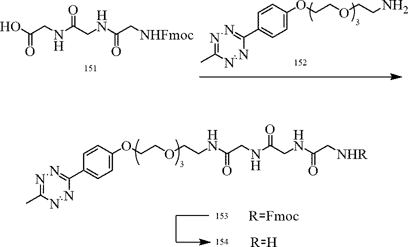

### Example 29. Synthesis of Compound 153

To a mixture of Fmoc—Gly—Gly—Gly—OH (151) (31.2 mg, 75.8 µmol) in anhydrous DMF (1 mL) were added N,N-diisopropylethylamine (40 µL, 29 mg, 0.23 mmol) and HATU (30.3 mg, 79.6 µmol). After 10 min tetrazine-PEG3-ethylamine (152) (30.3 mg, 75.8 µmol) was added and the mixture was vortexed. After 2 h, the mixture was purified by RP HPLC (C18, 30% → 90% MeCN (1% AcOH) in water (1% AcOH). The desired product was obtained as a pink film (24.1 mg, 31.8 µmol, 42%). LCMS (ESI+) calculated for C₃₈H₄₅N8O₉⁺(M+H⁺) 757.33 found 757.46.

### Example 30. Synthesis of Compound 154

To a solution of 153 (24.1 mg, 31.8 µmol) in DMF (500 µL) was added diethylamine (20 µL, 14 mg, 191 µmol). The mixture was left standing for 2 h and purified by RP HPLC (C18, 5% → 90% MeCN (1% AcOH) in water (1% AcOH). The desired product 154 was obtained as a pink film (17.5 mg, 32.7 µmol, quant). LCMS (ESI+) calculated for C₂₃H₃₅N8O₇⁺ (M+H⁺) 535.26 found 535.37.

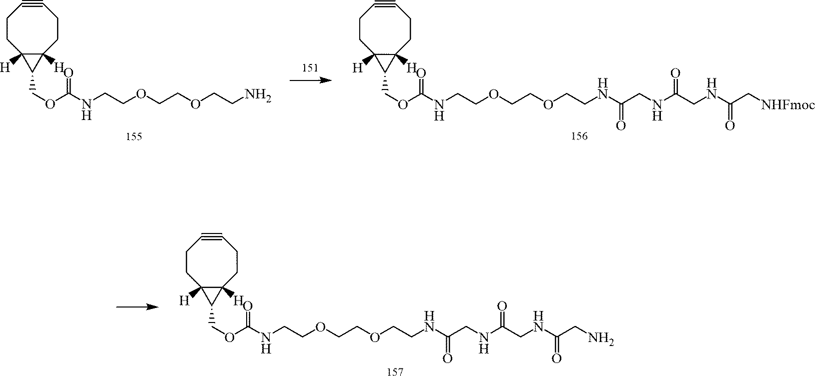

### Example 31. Synthesis of Compound 156

A solution of N-[(1R,8S,9 s)-bicyclo[6.1.0]non-4-yn-9-ylmethyloxycarbonyl]-1,8-diamino-3,6-dioxaoctane (155) (68 mg, 0.21 mmol) in dry DMF (2 mL) was transferred to a solution of Fmoc— Gly—Gly—Gly—OH (151) (86 mg, 0.21 mmol) in dry DMF (2 mL). DIPEA (100 µL, 0.630 mmol) and HATU (79 mg, 0.21 mmol) were added. After 1.5 h, the mixture was concentrated and the residue was purified by silica gel column chromatography (0 → 11% MeOH in DCM) which gave the desired compound 156 in 34% yield (52 mg, 0.072 mmol). LCMS (ESI+) calculated for C₃₅H₄₇N₅O₉⁺ (M+ H+) 717.34 found 718.39.

### Example 32. Synthesis of Compound 157

Compound 156 (21 mg, 0.029 mmol) was dissolved in DMF (2.4 mL) and piperidine (600 µL) was added. After 20 minutes, the mixture was concentrated and the residue was purified by preparative HPLC, which gave the desired compound 157 as a white solid (9.3 mg, 0.018 mmol, 64%). LCMS (ESI+) calculated for C₂₃H₃₇N₅O₇⁺(M+H⁺)495.27 found 496.56.

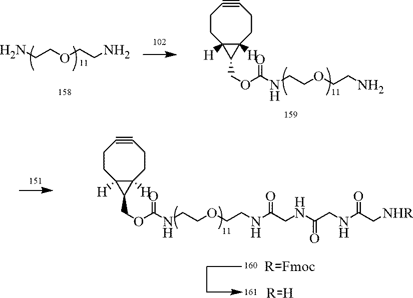

### Example 33. Synthesis of Compound 159

To a solution of amino-PEG₁₁-amine (158) (143 mg, 0.260 mmol) in DCM (5 mL) was slowly added (1R,8S,9 s)-bicyclo[6.1.0]non-4-yn-9-ylmethyl (4-nitrophenyl) carbonate (102) (41 mg, 0.13 mmol) dissolved in DCM (5 mL). After 1.5 h, the mixture was reduced and the residue was purified by silica gel column chromatography (0 → 20% 0.7 N NH₃ MeOH in DCM) which gave the desired compound 159 as a clear oil (62 mg, 0.086 mmol, 66%). LCMS (ESI+) calculated for C₃₅H₄₆N2O₁₃⁺(M+ H⁺)720.44 found 721.56.

### Example 34. Synthesis of Compound 160

A solution of 159 (62 mg, 0.086 mmol) in dry DMF (2 mL) was transferred to a solution of Fmoc— Gly—Gly—Gly—OH (151) (36 mg, 0.086 mmol) in dry DMF (2 mL). DIPEA (43 µL, 0.25 mmol) and HATU (33 mg, 0.086 mmol) were added. After 18 h, the mixture was concentrated and the residue was purified by silica gel column chromatography (0 → 20% MeOH in DCM) which gave the desired compound 160 in 62% yield (60 mg, 0.054 mmol). LCMS (ESI+) calculated for C₅₆H₈₃N5O₁₈⁺(M+H⁺) 1113.57 found 1114.93.

### Example 35. Synthesis of Compound 161

Compound 160 (36 mg, 0.032 mmol) was dissolved in DMF (2 mL) and piperidine (200 µL) was added. After 2 h, the mixture was concentrated and the residue was purified by silica gel column chromatography (0 → 40% 0.7 N NH₃ MeOH in DCM) which gave the desired compound 161 as a yellow oil (16.7 mg, 0.0187 mmol, 58%). LCMS (ESI+) calculated for C₄₁H₇₃N₅O₁₆⁺(M+H⁺) 891.51 found 892.82.

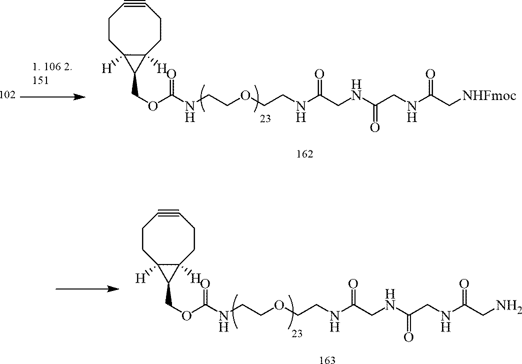

### Example 36. Synthesis of Compound 162

To a solution of amino-PEG23-amine (106) (60 mg, 0.056 mmol) in DCM (3 mL) was slowly added (1R,8S,9 s)-bicyclo[6.1.0]non-4-yn-9-ylmethyl (4-nitrophenyl) carbonate (102) (12 mg, 0.037 mmol) dissolved in DCM (5 mL). After 4 h, the mixture was concentrated and redissolved in DMF (2 mL), after which Fmoc—Gly—Gly—Gly—OH (51) (23 mg, 0.056 mmol), HATU (21 mg, 0.056 mmol), and DIPEA (27 µL, 0.16 mmol) were added. After 20 h, the mixture was concentrated and the residue was purified by silica gel column chromatography (0 → 27% MeOH in DCM) which gave the desired compound 162 in 93% (57 mg, 0.043 mmol). LCMS (ESI+) calculated for C₈₀H₁₃₁N₅O₃₀⁺ (M+NH₄⁺) 1641.89 found 1659.92.

### Example 37. Synthesis of Compound 163

Compound 162 (57 mg, 0.034 mmol) was dissolved in DMF (1 mL) and piperidine (120 µL) was added. After 2 h, the mixture was concentrated, redissolved in water and the Fmoc-piperidine byproduct was removed with extraction with diethyl ether (3 × 10 mL). After freeze dry, 163 was obtained as an yellow oil (46.1 mg, 0.032 mmol, 95%). LCMS (ESI+) calculated for C₆₅H₁₂₁N₅O₂₈⁺ (M+H) 1419.82 found 1420.91.

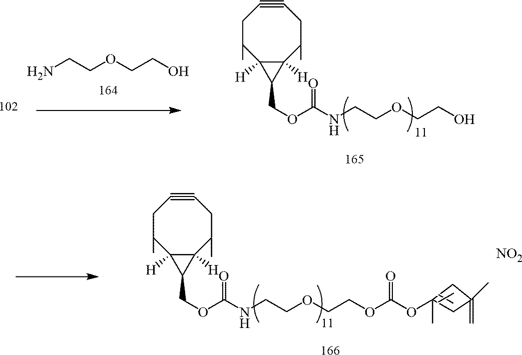

### Example 38. Synthesis of Compound 165

To a solution of (1R,8S,9 s)-bicyclo[6.1.0]non-4-yn-9-ylmethyl (4-nitrophenyl) carbonate (102) (204 mg, 0.650 mmol) were added amino-PEG12-alcohol (164) (496 mg, 0.908 mmol) and triethyl amine (350 µL, 2.27 mmol). After 19 h, the mixture was concentrated and the residue was purified by silica gel column chromatography (2 → 20% MeOH in DCM) which gave 165 as a clear yellow oil (410 mg, 0.560 mmol, 87%). LCMS (ESI+) calculated for C₃₅H₆₃NO₁₄⁺(M+Na⁺)721.42 found 744.43.

### Example 39. Synthesis of Compound 166

To a solution of 165 (410 mg, 0.560 mmol) in DCM (6 mL) were added 4-nitrophenyl chloroformate (171, 0.848 mmol) and triethyl amine (260 µL, 1.89 mmol). After 18 h, the mixture was concentrated and the residue was purified by silica gel column chromatography (0 → 7% MeOH in DCM) which gave the desired compound 166 as a clear oil (350 mg, 0.394 mmol, 70%). LCMS (ESI+) calculated for C₄₂H₆₆N₂O₁₈⁺(M+Na⁺) 886.43 found 909.61.

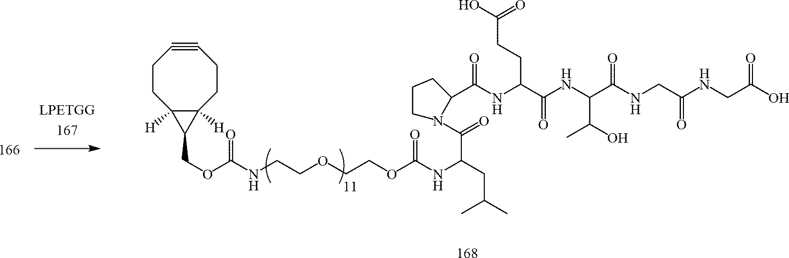

### Example 40. Synthesis of Compound 168

To a solution of 166 (15 mg, 0.017 mmol) in DMF (2 mL) were added peptide LPETGG (167) (9.7 mg, 0.017 mmol) and triethylamine (7 µL, 0.05 mmol). After 46 h, the mixture was concentrated and the residue was purified by preparative HPLC, which gave the desired compound 168 in 63% (14 mg, 0.010 mmol). LCMS (ESI+) calculated for C₆₀H₁₀₁N₇O₂₅⁺(M+H⁺) 1319.68 found 1320.92.

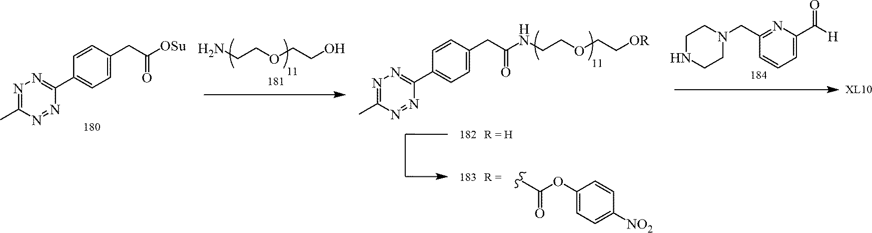

### Example 42. Synthesis of 182

To a solution of 180 (methyltetrazine-NHS ester, 19 mg, 0.058 mmol) in DCM (0.8 mL) were added 181 (33.6 mg, 0.061 mmol) and Et₃N (24 µL, 0.17 mmol). After stirring at room temperature for 2.5 h, the mixture was concentrated in vacuo and purified by flash column chromatography over silicagel (0 → 15% MeOH in DCM) which gave the desired compound 182 in 93% yield (41 mg, 0.054 mmol). LCMS (ESI+) calculated for C₃₅H₆₀N₅O₁₃⁺(M+H⁺) 758.88 found 758.64.

### Example 43. Synthesis of 183

To a solution of 182 (41 mg, 0.054 mmol) in DCM (3 mL) were added 4-nitrophenyl chloroformate (16 mg, 0.081 mmol) and Et₃N (23 µL, 0.16 mmol). After stirring at room temperature for 21 h, the mixture was concentrated in vacuo and purified by flash column chromatography over silicagel (gradient: A. 0% → 20% EtOAc in DCM (till p-nitrophenol was eluded), followed by gradient B. 0% → 13% MeOH in DCM) which gave the desired compound 183 in 76% yield (37.9 mg, 0.041 mmol). LCMS (ESI+) calculated for C₄₂H₆₃N₆O₁₇⁺ (M+H) 923.98 found 923.61.

### Example 44. Synthesis of XL10

To a solution of 184 (5.6 mg, 0.023 mmol), prepared according to MacDonald et al., Nat. Chem. Biol. 2015, 11, 326-334, incorporated by reference, in anhydrous DMF (0.1 mL) were added 183 (14.3 mg, 0.015 mmol) dissolved in anhydrous DMF (0.3 ml) and Et₃N (7 µL, 0.046 mmol). After stirring at room temperature for 2 h, the mixture was concentrated in vacuo and purified by flash column chromatography over silicagel (0 → 15% MeOH in DCM) which gave the desired compound XL10 in 50% yield (7.5 mg, 0.0076 mmol). LCMS (ESI+) calculated for C₄₇H₇₃N8O₁₅⁺(M+H⁺) 990.13 found 990.66.

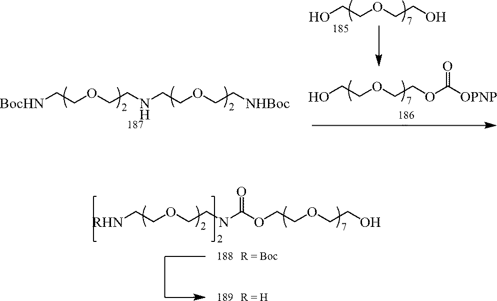

### Example 45. Synthesis of 186

To a solution of octa-ethylene glycol 185 in DCM (10 mL) was added triethylamine (1.0 mL, 7.24 mmol, 2.5 equiv.) followed by dropwise addition of a 4-nitrophenyl chloroformate (0.58 g, 2.90 mmol, 1 equiv.) solution in DCM (5 mL) in 28 minutes. After stirring the mixture for 90 minutes, it was concentrated in vacuo. The residue was purified by silicagel column chromatography (75% → 0% EtOAc in DCM followed by 0% → 7% MeOH in DCM). The product 186 was obtained in 38% yield as a colorless oil (584.6 mg, 1.09 mmol). LCMS (ESI+) calculated for C₂₃H₃₈NO₁₃⁺(M+H⁺) 536.23, found 536.93. ¹H-NMR (400 MHz, CDCl₃): δ (ppm) 8.28 (d, J = 12.0 Hz, 2H), 7.40 (d, J =12.0 Hz, 2H), 4.47 - 4.42 (m, 2H), 3.84 - 3.79 (m, 2H), 3.75 - 3.63 (m, 26H), 3.63 - 3.59 (m, 2H), 2.70 -2.55 (bs, 1H).

### Example 46. Synthesis of 188

To a solution of 187 (BocNH-PEG₂)₂NH, 202 mg, 0.42 mmol) in DCM (1 mL) was added part (0.5 mL, 0.54 mmol 1.3 equiv.) of a prepared stock solution of 186 (584 mg in DCM (1 mL)) followed by triethylamine (176 µL, 1.26 mmol, 3 equiv.) and HOBt (57 mg, 0.42 mmol, 1 equiv.). After stirring the mixture for 8 days, it was concentrated in vacuo. The residue was taken up in a mixture of acetonitrile (4.2 mL) and 0.1 N NaOH(_{aq}) (4.2 mL, 1 equiv.) and additional amount of solid NaOH (91.5 mg). After stirring the mixture for another 21.5 hours the mixture was extracted with DCM (3x 40 mL). The combined organic layers were concentrated in vacuo and the residue was purified by silicagel column chromatography (0% → 15% MeOH in DCM). Product 188 was obtained in 87% yield as a pale yellow oil (320.4 mg, 0.37 mmol). LCMS (ESI+) calculated for C₃₉H₇₈N₃O₁₈⁺(M+H⁺) 876.53, found 876.54.

H-NMR (400 MHz, CDCl₃): δ (ppm) 5.15 - 5.02 (bs, 2H), 4.25 - 4.19 (m, 2H), 3.76 - 3.46 (m, 50H), 3.35 - 3.26 (m, 4H), 2.79 - 2.69 (br. s, 1H), 1.44 (s, 18H).

### Example 47. Synthesis of 189

188 (320 mg, 0.37 mmol) was dissolved in DCM (1 mL). Then 4 M HCI in dioxane (456 µL, 1.83 mmol, 5 equiv.) was added. After stirring the mixture for 3.5 hours, additional 4 M HCI in dioxane (450 µL, 1.80 mmol, 4.9 equiv.) was added. After stirring the mixture for another 3.5 hours, additional 4 M HCI in dioxane (450 µL, 1.80 mmol, 4.9 equiv.) was added. After stirring the mixture for 16.5 hours the mixture was concentrated in vacuo. Product 189 was obtained in quantitative yield as a white sticky solid. This was used directly in the next step. H-NMR (400 MHz, DMSO-d6): δ (ppm) 8.07 - 7.81 (bs, 6H), 4.15 - 4.06 (m, 2H), 3.75 - 3.66 (m, 2H), 3.65 - 3.48 (m, 48H), 3.03 - 2.92 (m, 4H).

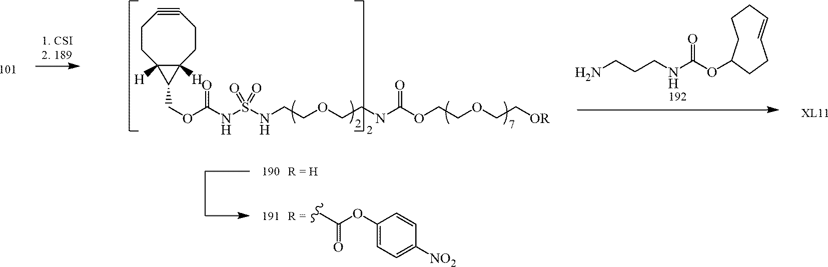

### Example 48. Synthesis of 190

To a solution of BCN-OH (101, 164 mg, 1.10 mmol, 3 equiv.) in DCM (3 mL) was added CSI (76 µL, 0.88 mmol, 2.4 equiv.). After stirring for 15 minutes triethylamine (255 µL, 5.50 mmol, 5 equiv.) was added. A solution of 189 was prepared by adding DCM (3 mL) and triethylamine (508 µL, 11.0 mmol, 10 equiv.). This stock solution was added to the original reaction mixture after 6 minutes. After stirring the mixture for 21.5 hours, it was concentrated in vacuo. The residue was purified by silicagel column chromatography (0% → 10% MeOH in DCM). Product 190 was obtained in 39% yield as pale yellow oil (165.0 mg, 139 µmol). LCMS (ESI+) calculated for C₄₃H₇₂N₅O₁₈S₂⁺(M+H⁺) 1186.54, found 1186.65.

¹H-NMR (400 MHz, CDCl₃): δ (ppm) 6.09 - 5.87 (m, 2H), 4.31 - 4.19 (m, 6H), 3.76 - 3.50 (m, 50H), 3.40 - 3.29 (m, 4H), 2.38 - 2.16 (m, 12H), 1.66 - 1.47 (m, 4H), 1.40 (quintet, J = 8.0 Hz, 2H), 1.04 - 0.94 (m, 4H).

### Example 49. Synthesis of 191

To a solution of 190 (101 mg, 0.085 mmol) in DCM (2.0 mL) were added bis(4-nitrophenyl) carbonate (39 mg, 0.127 mmol) and Et₃N (36 uL, 0.25 mmol). After stirring at room temperature for 42 h, the crude mixture was concentrated in vacuo and purified by flash column chromatography over silicagel (A. 0% → 25% EtOAc in DCM (till p-nitrophenol was eluded), followed by gradient B. 0% → 12% MeOH in DCM) to give 191 as a clear oil (49 mg, 0.036 mmol, 42%). LCMS (ESI+) calculated for C₅₈H₉₁N₆O₂₆S₂⁺ (M+H⁺) 1352.50 found 1352.78.

### Example 50. Synthesis of XL11

To a solution of 191 (7 mg, 0.0059 mmol) in anhydrous DMF (130 µL) were added Et₃N (2.2 uL, 0.015 mmol) and TCO-amine hydrochloride (Broadpharm) (1.8 mg, 0.0068 mmol). After stirring at room temperature for 19 h, the crude mixture was purified by flash column chromatography over silicagel (0% → 15% MeOH in DCM) to give XL11 as a clear oil (1.5 mg, 0.001 mmol, 17%). LCMS (ESI+) calculated for C₆₄H₁₁₁N₈O₂₅S₂⁺ (M+NH₄⁺) 1456.73 found 1456.81.

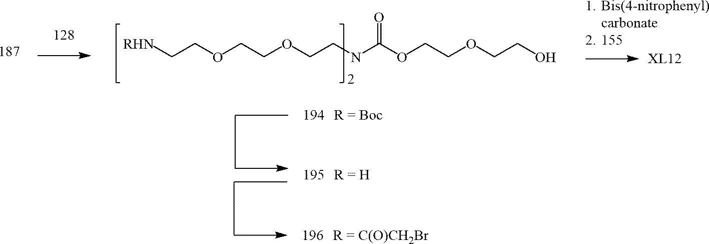

### Example 51. Synthesis of 194

To a solution of available 187 (638 mg, 1.33 mmol) in DCM (8.0 mL) were added 128 (470 mg, 1.73 mmol), Et₃N (556.0 µL, 4.0 mmol), and 1-hydroxybenzotriazole (179.0 mg, 1.33 mmol). After stirring for 41 h at ambient temperature, the mixture was concentrated in vacuo and redissolved in MeCN (10 mL) followed by the addition of aqueous 0.1 M NaOH solution (10 mL) and solid NaOH pellets (100.0 mg). After 1.5 h, DCM (20 mL) was added and the desired compound was extracted four times. The organic layers were concentrated in vacuo and the residue was purified by flash column chromatography over silicagel (0% → 12% MeOH in DCM) to give 194 as a clear yellow oil (733 mg, 1.19 mmol, 90%). ¹H NMR (400 MHz, CDCl₃) δ (ppm) 4.29 - 4.23 (m, 2H), 3.77 - 3.68 (m, 4H), 3.65 - 3.56 (m, 14H), 3.56 - 3.49 (m, 8H), 3.37 - 3.24 (m, 4H), 1.45 (s, 18H). LCMS (ESI+) calculated for C₂₇H₅₄N₃O₁₂⁺ (M+H) 612.73 found 612.55.

### Example 52. Synthesis of 195

To a solution of 194 (31.8 mg, 0.052 mmol) in DCM (1.0 mL) was added 4.0 M HCI in dioxane (0.4 mL). After stirring for 2.5 h at ambient temperature, the reaction mixture was concentrated in vacuo and in between redissolved in DCM (2 mL) and concentrated. Compound 195 was obtained as a clear oil in quantitative yield. LCMS (ESI+) calculated for C₁₇H₃₈N₃O₈⁺(M+H⁺) 412.50 found 412.45

### Example 53. Synthesis of 196

To a cold solution (0° C.) of 195 (21.4 mg, 0.052 mmol) in DCM (1.0 mL) were added Et₃N (36 µL, 0.26 mmol) and 2-bromoacetyl bromide (10.5 µL, 0.12 mmol). After stirring for 10 min on ice, the ice bath was removed and aqueous 0.1 M NaOH solution (0.8 mL) was added. After stirring at room temperature for 20 min, the water layer was extracted with DCM (2x 5 mL). The organic layers were combined and concentrated in vacuo. The crude brown oil was purified by flash column chromatography over silicagel (0% → 18% MeOH in DCM) to give 196 as a clear oil (6.9 mg, 0.011 mmol, 20%). LCMS (ESI+) calculated for C₂₁H₄₀Br₂N₃O₁₀⁺ (M+H⁺) 654.36 found 654.29.

### Example 54. Synthesis of XL12

To a solution of 196 (6.9 mg, 0.011 mmol) in DCM (0.8 mL) were added bis(4-nitrophenyl) carbonate (3.8 mg, 0.012 mmol) and Et₃N (5 µL, 0.03 mmol). After stirring at room temperature for 18 h, 155 (BCN-PEG₂-NH₂, 3.3 mg, 0.01 mmol) dissolved in DCM (0.5 mL) was added. After stirring for an additional of 2 h, the mixture was concentrated in vacuo and purified by flash column chromatography over silica gel (gradient: A. 0% → 30% EtOAc in DCM (till p-nitrophenol was eluded), followed by gradient B. 0% → 20% MeOH in DCM) to give XL12 as a clear oil (1.0 mg, 0.001 mmol, 9%). LCMS (ESI+) calculated for C₃₉H₆₆Br₂N₅O₁₅⁺ (M+H⁺) 1004.77 found 1004.51.

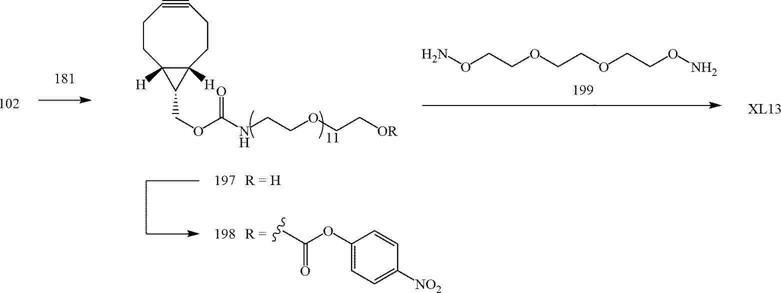

### Example 55. Synthesis of 197

To a solution of 102 (204 mg, 0.647 mmol) in DCM (20 mL) were added 181 (496 mg, 0.909 mmol) and Et₃N (350 µL, 2.27 mmol). After stirring at room temperature for 19 h, solvent was reduced in vacuo and the residue was purified by flash column chromatography over silicagel (2 → 20% MeOH in DCM) which gave the desired compound 197 as a yellow oil in 87% yield (410 mg, 0.567 mmol). LCMS (ESI+) calculated for C₃₅H₆₃NO₁₄Na⁺(M+Na⁺)744.86 found 744.43.

### Example 56. Synthesis of 198

To a solution of 197 (410 mg, 0.567 mmol) and 4-nitrophenyl chloroformate (172 mg, 0.853 mmol) in DCM (6 mL) was added Et₃N (260 µL, 1.88 mmol). After stirring at room temperature for 18 h, solvent was reduced in vacuo and the residue was purified by flash column chromatography over silicagel (0 → 7% MeOH in DCM) which gave the desired compound 198 as a clear oil in 70% yield (350 mg, 0.394 mmol). LCMS (ESI+) calculated for C₄₂H₆₆N₂O₁₈Na⁺(M+Na⁺) 909.96 found 909.61.

### Example 57. Synthesis of XL13

To a solution of 198 (44.2 mg, 0.05 mmol) in DCM (5 mL) were added 199 (bis-aminooxy-PEG₂, 33.3 mg, 0.18 mmol) and Et₃N (11 µL, 0.07 mmol). After stirring at room temperature for 67 h, the mixture was concentrated in vacuo and purified by RP HPLC (Column Xbridge prep C18 5 um OBD, 30x100 mm, 5% → 90% MeCN in H₂O (both containing 1% acetic acid)). The product XL13 was obtained as a clear oil (8.1 mg, 0.0087 µmol, 17%). LCMS (ESI+) calculated for C₄₂H₇₈N₃O₁₉⁺ (M+H⁺) 929.08 found 928.79.

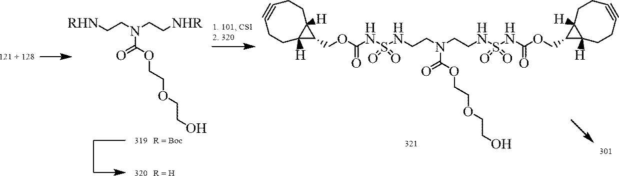

### Example 58. Synthesis of 319

To a solution of compound 121 (442 mg, 1.46 mmol) in DCM (1 mL) and DMF (200 µL) was added a solution of compound 128 in DCM (1 mL) and triethylamine (609 µL, 4.37 mmol). After stirring the mixture for 16 hours, it was concentrated in vacuo. The residue was purified by silica gel column chromatography (50% → 100% EtOAc in heptane) and gave 319 (316 mg) This was further purified by RP HPLC (Column Xbridge prep C18 5 µm OBD, 30x100 mm, 5% → 90% MeCN (1% AcOH) in water (1% AcOH). Product 319 was obtained in 17% yield as a colorless oil (110 mg, 0.25 mmol). LCMS (ESI+) calculated for C₁₉H₃₇N₃NaO₈⁺ (M+Na⁺) 458.25, found 458.33. H-NMR (400 MHz, CDCl₃): δ (ppm) 5.41 - 4.89 (m, 2H), 4.31 - 4.24 (m, 2H), 3.78 - 3.68 (m, 4H), 3.65 - 3.59 (m, 2H), 3.44 - 3.34 (m, 4H), 3.34 - 3.19 (m, 4H), 1.43 (s, 18H).

### Example 59. Synthesis of 320

Compound 319 (107 mg, 0.25 mmol) was dissolved in DCM (1 mL). Then 4 M HCI in dioxane (300 µL, 1.2 mmol, 4.8 equiv.) was added. After stirring the mixture for 15 hours, it was decanted from the precipitate and the precipitate was washed once with DCM (2 mL). Product 320 was obtained in quantitive yield as a white sticky solid (89.9 mg, 0.29 mmol). This was used directly in the next step.

### Example 60. Synthesis of 321

To a solution of 101 (75 mg, 0.50 mmol, 2 equiv.) in DCM (1 mL) was added CSI (41 µL, 0.48 mmol, 1.9 equiv.). After stirring for 6 minutes, triethylamine (139 µL, 1.0 mmol, 4 equiv.) was added. A stock solution of 320 was prepared by adding DMF (200 µL) and DCM (2 mL) followed by triethylamine (139 µL, 0.75 mmol, 3 equiv.). Part of this stock solution of 320 (32 µL, 0.25 mmol) was added to the original reaction mixture containing the CSI. After stirring the mixture for 16 hours, it was concentrated in vacuo. The residue was purified by silica gel column chromatography (0% → 10% MeOH in DCM). Product 321 was obtained in 3% yield as a colorless oil (11 mg, 14.2 µmol). LCMS (ESI+) calculated for C₃₁H₄₈N₅O₁₂S₂⁺ ((M+H⁺) 746.27, found 746.96. H-NMR (400 MHz, CDCl₃): δ (ppm) 6.36 - 5.94 (m, 2H), 4.38 - 4.17 (m, 6H), 3.84 - 3.79 (m, 2H), 3.77 - 3.72 (m, 2H), 3.68 - 3.63 (m, 2H), 3.54 - 3.45 (m, 4H), 3.39 - 3.27 (m, 4H), 2.38 - 2.16 (m, 12H), 1.67 - 1.47 (m, 5H), 1.40 (quintet, J = 8.0 Hz, 2H), 1.05 - 0.93 (m, 4H).

### Example 61. Synthesis of 301 (LD01)

To a solution of 321 (10.6 mg, 14.2 µmol) in DCM (100 µL) were added bis(4-nitrophenyl) carbonate (4.3 mg, 14.2 µmol, 1.0 equiv.) and triethylamine (5.9 µL, 42.6 µmol, 3.0 equiv.). After stirring for 66 hours part of this mixture was treated with a stock solution of vc-PABC-MMAE.TFA in DMF (200 µL, 50 mg/mL) and an additional amount of triethylamine (5.9 µL, 42.6 µmol, 3.0 equiv.). After 24 hours it was concentrated partly in vacuo. The residue was purified by RP HPLC (Column Xbridge prep C18 5 µm OBD, 30x100 mm, 5% → 90% MeCN (1% AcOH) in water (1% AcOH). Compound 301 was obtained in 28% yield as a film (3.4 mg, 1.9 µmol). LCMS (ESI+) calculated for C₉₀H₁₄₀N₁₅O₂₅S₂⁺((M+H⁺) 1894.96, found 1895.00.

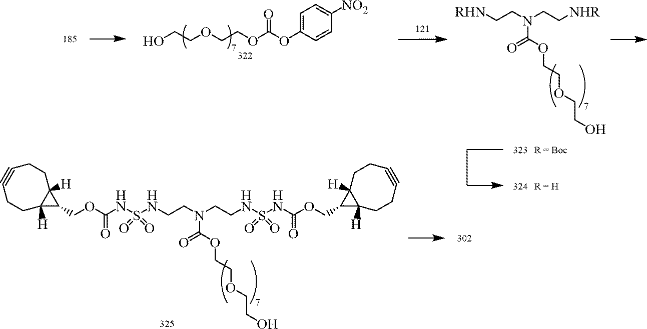

### Example 62. Synthesis of 322

To a solution of 185 (octaethylene glycol) in DCM (10 mL) was added triethylamine (1.0 mL, 7.24 mmol; 2.5 equiv.) followed by dropwise addition of a 4-nitrophenyl chloroformate (0.58 g; 2.90 mmol; 1 equiv.) solution in DCM (5 mL) in 28 minutes. After stirring the mixture for 90 minutes, it was concentrated in vacuo. The residue was purified by silica gel column chromatography (75% → 0% EtOAc in DCM followed by 0% → 7% MeOH in DCM). Product 322 was obtained in 38% yield as a colorless oil (584.6 mg; 1.09 mmol). LCMS (ESI+) calculated for C₂₃H₃₈NO₁₃⁺(M+H⁺)536.23, found 536.93. ¹H-NMR (400 MHz, CDCl₃): δ (ppm) 8.28 (d, J = 12.0 Hz, 2H), 7.40 (d, J = 12.0 Hz, 2H), 4.47 - 4.42 (m, 2H), 3.84 - 3.79 (m, 2H), 3.75 - 3.63 (m, 26H), 3.63 - 3.59 (m, 2H), 2.70 - 2.55 (br. s, 1H).

### Example 63. Synthesis of 323

To a solution of compound 121 (127 mg, 0.42 mmol) in DCM (1 mL) was added part (0.5 mL; 0.54 mmol; 1.3 equiv.) of a prepared stock solution of 322 (584 mg in DCM (1 mL)) followed by triethylamine (176 µL, 1.26 mmol; 3 equiv.) and HOBt (57 mg; 0.42 mmol; 1 equiv.). After stirring the mixture for 4.5 days, it was concentrated in vacuo. The residue was taken up in a mixture of acetonitrile (4.2 mL) and 0.1 N NaOH (4.2 mL, 1 equiv.). After stirring the mixture for 24 hours, additional solid NaOH (104.5 mg) was added. After stirring the mixture for another 5 hours, the mixture was extracted with DCM (2 x 10 mL). The combined organic layers were concentrated in vacuo and the residue was purified by silica gel column chromatography (0% → 15% MeOH in DCM). Product 323 was obtained in 54% yield as a pale yellow oil (164.5 mg, 0.23 mmol). LCMS (ESI+) calculated for C₂₆H₅₄N3O₁₂⁺(M-BOC⁺) 600.36, found 600.49. H-NMR (400 MHz, CDCl₃): δ (ppm) 5.27 - 5.05 (m, 2H), 4.26 - 4.21 (m, 2H), 3.76 - 3.59 (m, 30H), 3.43 - 3.33 (m, 4H), 3.33 -3.22 (m, 4H), 1.43 (s, 18H).

### Example 64. Synthesis of 324

Compound 323 (164 mg, 0.23 mmol) was dissolved in DCM (1 mL). Then 4 M HCI in dioxane (293 µL, 1.17 mmol, 5 equiv.) was added. After stirring the mixture for 18 hours, additional 4 M HCI in dioxane (293 µL, 1.17 mmol, 5 equiv.) was added. After stirring the mixture for another 5 hours, the mixture was concentrated in vacuo. Product 324 was obtained in quantitative yield as a white sticky solid (132 mg, 0.23 mmol). This was used directly in the next step.

### Example 65. Synthesis of 325

To a solution of 101 (81 mg, 0.54 mmol, 2.3 equiv.) in DCM (2 mL) was added CSI (43 µL, 0.49 mmol, 2.1 equiv.). After stirring for 15 minutes triethylamine (164 µL, 1.17 mmol, 5 equiv.) was added. A solution of 324 was prepared by adding DCM (2 mL) and triethylamine (164 µL, 1.17 mmol, 5 equiv.). This stock solution was added to the original reaction mixture after 6 minutes. After stirring the mixture for 23 hours, it was concentrated in vacuo. The residue was purified by silica gel column chromatography (0% → 12% MeOH in DCM). Product 325 was obtained in 31% yield as pale yellow oil (73.0 mg, 72.2 µmol). LCMS (ESI+) calculated for C₄₃H₇₂N₅O₁₈S₂⁺(M+H⁺)1010.43, found 1010.50.

¹H-NMR (400 MHz, CDCl₃): δ (ppm) 6.21 - 5.85 (m, 2H), 4.38 - 4.17 (m, 6H), 3.80 - 3.57 (m, 30H), 3.57 - 3.44 (m, 4H), 3.44 - 3.30 (m, 4H), 2.38 - 2.16 (m, 12H), 1.64 - 1.48 (m, 4H), 1.40 (quintet, J = 8.0 Hz, 2H), 1.05 - 0.91 (m, 4H).

### Example 66. Synthesis of 302 (LD02)

To a solution of 325 (19.5 mg, 19.7 µmol) in DCM (100 µL) were added bis(4-nitrophenyl) carbonate (6.0 mg, 19.7 µmol, 1.0 equiv.) and triethylamine (8.2 µL, 59.1 µmol, 3.0 equiv.). After stirring for 66 hours part of this mixture was treated with a stock solution of vc-PABC-MMAE.TFA in DMF (200 µL, 50 mg/mL) and an additional amount of triethylamine (8.2 µL, 59.1 µmol, 3.0 equiv.). After 95 hours it was concentrated partly in vacuo. The residue was purified by RP HPLC (Column Xbridge prep C18 5 µm OBD, 30x100 mm, 5% → 90% MeCN (1% AcOH) in water (1% AcOH). Compound 302 was obtained in 9% yield as a film (3.7 mg, 1.71 µmol). LCMS (ESI+) calculated for C₁₀₂H₁₆₅N₁₅O₃₁S₂²⁺(M+2H⁺) 1080.56, found 1080.74.

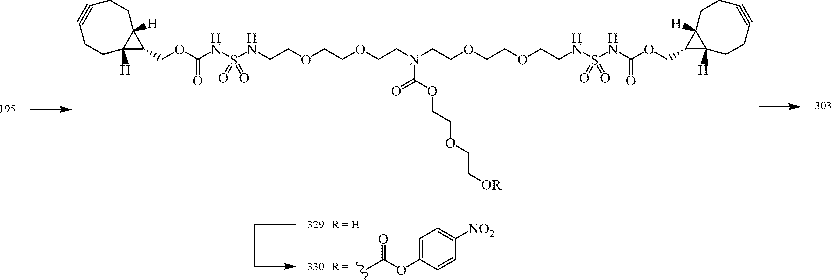

### Example 67. Synthesis of 329

To a solution of 101 (18 mg, 0.12 mmol) in DCM (1 mL) was added chlorosulfonyl isocyanate (CSI). After 30 min, Et₃N (37 µL, 27 mg, 0.27 mmol) was added. To a solution of 195 (26 mg, 0.054 mmol) in DCM (1 mL) was added Et₃N (37 µL, 27 mg, 0.27 mmol). This mixture was added to the reaction mixture. After 45 min, the reaction mixture was concentrated and the residue was purified by silica gel chromatography (DCM to 7% MeOH in DCM). Product 329 was obtained as a colorless film (27 mg, 0.029 mmol, 54%). LCMS (ESI+) calculated for C₃₉H₆₄N₅O₁₆S₂⁺ (M+H⁺) 922.38, found 922.50.

### Example 68. Synthesis of 330

To a solution of 329 in DCM (1 mL) was added bis(4-nitrophenyl) carbonate (8.9 mg, 29.3 µmol) and Et₃N (12.2 µL, 8.9 mg, 87.9 µmol). After 1 d, 0.28 mL was used for the preparation of compound 303. After 2 d, extra bis(4-nitrophenyl) carbonate (7.0 mg, 23 µmol) was added to the main reaction mixture. After 1 day, the reaction mixture was concentrated and the residue was purified by silica gel column chromatography. Product 330 was was obtained as a colorless film (17.5 mg, 0.016 mmol, 55% (76% corrected)). LCMS (ESI+) calculated for C₄₆H₆₇N₆O₂₀S₂⁺ (M+H⁺) 1087.38, found 1087.47.

### Example 69. Synthesis of 303 (LD03)

To the reaction mixture of 330 (0.28 mL, theoretically containing 8.8 mg, 8.1 µmol) was added Et₃N (3.4 µL, 2.5 mg, 24.3 µmol) and a solution of vc-PABC-MMAE.TFA (10 mg, 8.1 µmol) in DMF (200 µL). After 21 h, 2.2′-(ethylenedioxy)bis(ethylamine) (4.7 µL, 4.8 mg, 32 µmol) was added. After 45 min, the reaction mixture was concentrated under a stream of nitrogen gas. The residue was purified by RP-HPLC (Column Xbridge prep C18 5 µm OBD, 30x100 mm, 30% to 90% MeCN (1% AcOH) in water (1% AcOH). Product 303 was obtained as a colorless film (5.6 mg, 2.7 µmol). LCMS (ESI+) calculated for C₉₈H₁₅₇N₁₅O₂₉S₂²⁺((M+2H⁺)/2) 1036.53, found 1036.70.

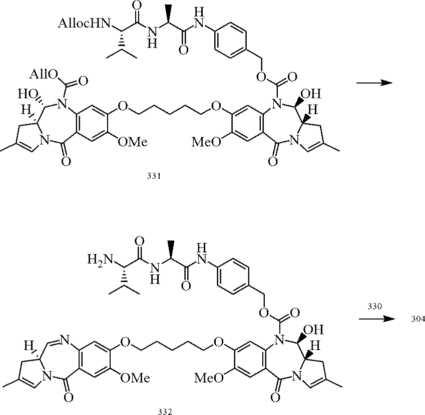

### Example 70. Synthesis of 332

To a solution of Alloc₂-va-PABC-PBD 331 (10.0 mg, 0.009 mmol) in degassed DCM (400 µL, obtained by purging N₂ through DCM for 5 minutes) were added pyrrolidine (1.9 µL, 0.027 mmol) and Pd(PPh₃)₄ (1.6 mg, 0.0014 mmol). After stirring for 15 min at ambient temperature, the reaction mixture was diluted with DCM (10 mL) and aqueous saturated NH₄Cl (10 mL) was added. The crude mixture was extracted with DCM (3 × 10 mL). The organic layers were combined, dried over Na₂SO₄, filtered, and concentrated in vacuo. The yellow residue was redissolved in DMF (450 µL) and MeCN (450 µL) and purified by RP HPLC (Column Xbridge prep C18 5 µm OBD, 30x100 mm, 5% → 90% MeCN in H₂O (both containing 0.1% formic acid)). The pure fractions were neutralized over a SPE column (PL-HCO₃ MP, 500 mg/6 mL), concentrated and co-evaporated with MeCN (2 × 5 mL) to give 332 as a white solid (4.8 mg, 0.005 mmol, 58%). LCMS (ESI+) calculated for C₄₉H₆₀N7O₁₁⁺ (M+H⁺) 923.04 found 923.61.

### Example 71. Synthesis of 304 (LD04)

To a solution of 332 (4.8 mg, 0.005 mmol) in anhydrous degassed DMF (60 µL, obtained by purging N₂ through DMF for 5 minutes) were added 330 (10 mg, 0.009 mmol, dissolved in 48 µL anhydrous degassed DMF), Et₃N (3.6 µL, 0.026 mmol), and HOBt (stock in anhydrous degassed DMF, 5.1 µL, 0.35 mg, 0.0026 mmol, 0.5 eq). After 41 h stirring at ambient temperature in the dark, the crude reaction mixture was diluted with DCM (300 µL) and purified by flash column chromatography over silicagel (0% → 12% MeOH in DCM) to give 304 as a clear yellow oil (4.0 mg, 0.0021 mmol, 41%). LCMS (ESI+) calculated for C₈₉H₁₂₁N₁₂O₂₈S₂⁺ (M+H) 1871.11 found 1871.09.

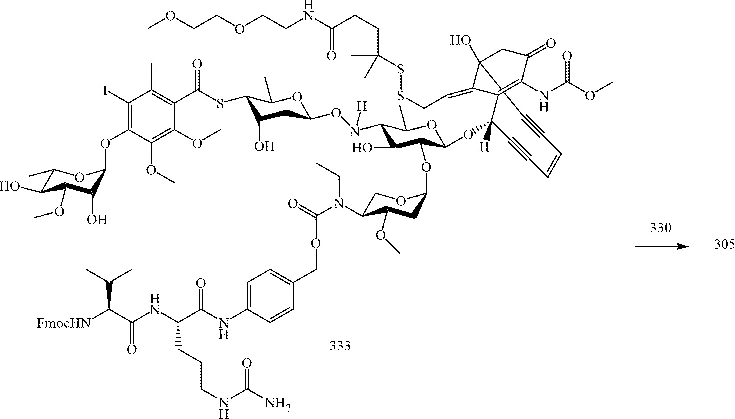

### Example 72. Synthesis of 305 (LD05)

To a solution of 333 (2.9 mg, 0.0013 mmol), prepared according to WO2019110725A1, Example 5-5, incorporated by reference, in anhydrous DMF (60 µL) were added 330 (1.45 mg, 0.0013 mmol) and Et₃N (1.2 µL, 0.023 mmol). After stirring at room temperature for 48 h, the reaction mixture was diluted with DMF (500 µL) and purified by RP HPLC (Column Xbridge prep C18 5 µm OBD, 30x100 mm, 30% → 100% MeCN in H₂O (both containing 1% acetic acid)). The product 305 was obtained as a colorless film (0.6 mg, 0.207 µmol, 16%). LCMS (ESI+) calculated for C₁₂₄H₁₈₂IN₁₄O₄₆S₅⁺ (M/2+H⁺) 1447.03 found 1447.19.

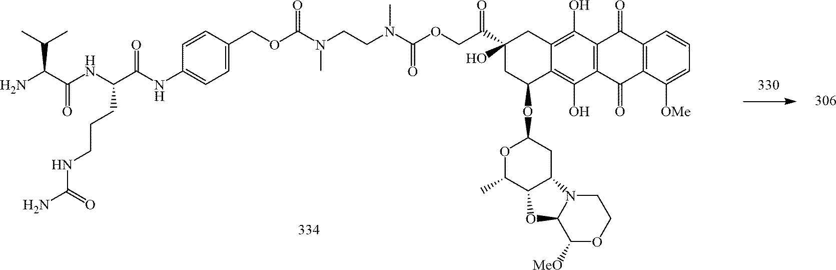

### Example 73. Synthesis of 306 (LD06)

To a solution of 330 (7 mg, 0.006 mmol) in anhydrous DMF (150 µL) were added a stock of vc-PABC-DMEA-PNU (334) in anhydrous DMF (125 µL, 5.7 mg, 0.005 mmol) and Et₃N (2 µL, 0.015 mmol). After stirring at room temperature for 25 h, the reaction mixture was diluted with DCM (0.3 mL) and purified by flash column chromatography over silica gel (0% → 20% MeOH in DCM) to give 306 as a red film (5 mg, 0.0024 mmol, 47%). LCMS (ESI+) calculated for C₉₆H₁₃₃N₁₃O₃₆S₃+(M/2+H⁺) 1055.64 found 1055.50.

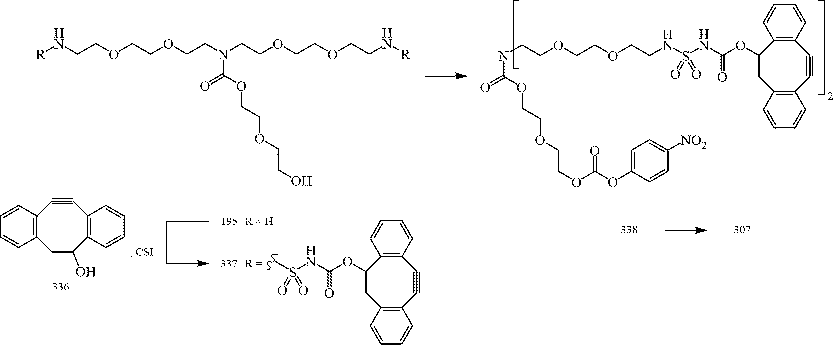

### Example 74. Synthesis of 337

Compound 336 (DIBO, 95 mg, 0.43 mmol) was dissolved in DCM (1.0 mL) and chlorosulfonyl isocyanate (33.0 µL, 0.37 mmol) was added at room temperature, and after 2 min insoluble material was formed. After stirring for an additional 15 min at room temperature, Et₃N (120.0 µL, 0.85 mmol) was added, all insoluble material disappeared, and addition of a mixture of 195 (71 mg, 0.0171) dissolved in DCM (1.0 mL) and Et₃N (120.0 µL, 0.85 mmol) was performed. After stirring at room temperature for 16 h, the crude mixture was concentrated in vacuo and purified by flash column chromatography over silicagel (0% → 15% MeOH in DCM) after which it was co-evaporated with EtOAc (2x) to completely remove the MeOH. Product 337 was obtained as a waxy white solid (136.0 mg, 0.12 mmol, 75%). LCMS (ESI+) calculated for C₅₁H₆₃N₆O₁₆S₂⁺ (M+NH₄⁺) 1080.21 found 1080.59.

### Example 75. Synthesis of 338

To a solution of 337 (136.0 mg, 0.12 mmol) in DCM (2.0 mL) were added bis-(4-nitrophenyl) carbonate (47.0 mg, 0.15 mmol) and Et₃N (54.0 µL, 0.38 mmol). After stirring at room temperature for 18 h, the crude mixture was concentrated in vacuo and purified by flash column chromatography over silicagel (gradient: A. 0% → 35% EtOAc in DCM (until p-nitrophenol was eluded), followed by gradient, B. 0% → 13% MeOH in DCM) to give 338 as a light-yellow oil (89.0 mg, 0.07 mmol, 60%). LCMS (ESI+) calculated for C₄₈H₆₆N₇O₂₀S₂⁺ (M+NH₄⁺) 1245.31 found 1245.64.

### Example 76. Synthesis of 307 (LD07)

To a solution of 338 (6.95 mg, 0.005 mmol) in anhydrous DMF (93.0 µL) were added Et₃N (2.4 µL, 0.017 mmol) and stock solution of vc-PABC-MMAE.TFA (Levena Bioscience) in anhydrous DMF (70 µL, 7.0 mg, 0.005 mmol). After stirring at room temperature for 18 h, DMF (450 µL) was added and the crude mixture was purified by RP HPLC (Column Xbridge prep C18 5 µm OBD, 30x100 mm, 30% → 100% MeCN in H₂O (both containing 1% acetic acid)). The product 307 was obtained as a colorless film (4.5 mg, 0.002 mmol, 36%). LCMS (ESI+) calculated for C₁₁₀H₁₅₂N₁₅O₂₉S₂⁺ (M/2+H) 1106.30 found 1106.79.

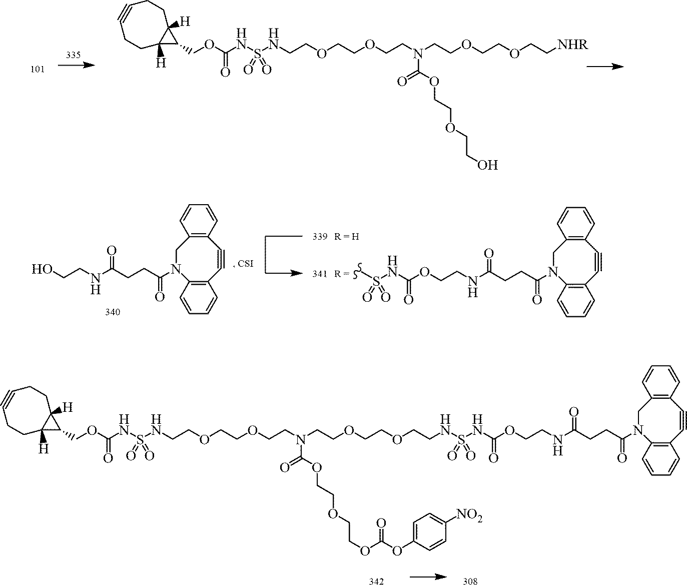

### Example 77. Synthesis of 341

Compound 101 (16.3 mg, 0.10 mmol) was dissolved in DCM (0.8 mL) and chlorosulfonyl isocyanate (8.6 µL, 0.099 mmol) was added at room temperature. After stirring for 15 min at room temperature, Et₃N (69.0 µL, 0.49 mmol) was added, followed by the addition of a mixture of 335 (40 mg, 0.099 mmol) dissolved in DCM (1.0 mL) and Et₃N (69.0 µL, 0.49 mmol). This mixture was stirred at room temperature for 1.5 h (mixture 1) to give crude 339. In another vial, 340 (DBCO-C₂-OH, Broadpharm) (34.0 mg, 0.099 mmol) was dissolved in DCM (0.8 mL) at room temperature and chlorosulfonyl isocyanate (7.75 µL, 0.089 mmol) was added. After stirring at room temperature for 15 min, Et₃N (69.0 µL, 0.49 mmol) was added followed by crude 339. After stirring at room temperature for another 2 h, the reaction mixture was concentrated in vacuo and purified by flash column chromatography over silicagel (0% → 15% MeOH in DCM) after which it was co-evaporated with EtOAC (2x) to completely remove the MeOH. Product 341 was obtained as a clear yellow oil (20.0 mg, 0.017 mmol, 17%). LCMS (ESI+) calculated for C₅₀H₇₀N₇O₁₈S₂⁺ (M+H⁺) 1121.26 found 1121.59.

### Example 78. Synthesis of 342

To a solution of 341 (20.0 mg, 0.17 mmol) in DCM (1.0 mL) were added bis(4-nitrophenyl) carbonate (5.6 mg, 0.019 mmol) and Et₃N (7.5 µL, 0.053 mmol). After stirring at room temperature for 40 h, the crude mixture was concentrated in vacuo and purified by flash column chromatography over silicagel (gradient: A. 0% → 30% EtOAc in DCM (until p-nitrophenol was eluded), followed by gradient B. 0% → 20% MeOH in DCM) to give 342 as a clear light yellow oil (6.9 mg, 0.005 mmol, 30%). LCMS (ESI+) calculated for C₅₇H₇₃N₈O₂₂S₂⁺ (M+H⁺) 1286.36 found 1286.57.

### Example 79. Synthesis of 308 (LD08)

To a solution of 342 (3.6 mg, 0.0028 mmol) in anhydrous DMF (35.0 µL) were added Et₃N (1.2 µL, 0.008 mmol) and stock solution of vc-PABC-MMAE.TFA (Levena Bioscience) in anhydrous DMF (34 µL, 3.4 mg, 0.0028 mmol). After stirring at room temperature for 27 h, DCM (400 µL) was added and the crude mixture was purified by flash column chromatography over silicagel (0% → 30% MeOH in DCM) to give 308 as a colorless film (3.7 mg, 0.0016 mmol, 58%). LCMS (ESI+) calculated for C₁₀₉H₁₆₁N₁₇O₃₁S₂⁺ (M/2+H⁺) 1135.84 found 1135.73.

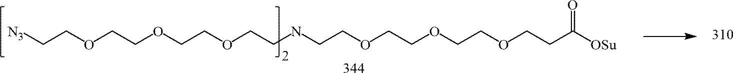

### Example 80. Synthesis of 310 (LD10)

To an Eppendorf vial containing 344 (4.3 mg, 6.0 µmol, 1.7 equiv.) was added was added a vc-PABC-MMAF.TFA salt in DMF (4.00 mg, 100 µL, 34.31 mmolar, 3.43 µmol, 1.0 equiv.), followed by triethylamine (1.43 µL, 10.3 µmol, 3.0 Eq). The mixture was mixed and the resulting colorless solution was left at rt for circa 3 hours. The reaction mixture was then purified directly via RP HPLC (Column Xbridge prep C18 5 µm OBD, 30x100 mm, 30% → 90% MeCN (1% AcOH) in water (1% AcOH). The desired product 310 was obtained as a colorless residue (4.5 mg, 2.7 µmol, 79% yield). LCMS (ESI+) calculated for C₈₀H₁₃₄N₁₅O₂₂⁺ (M+H⁺) 1656.98. found 1657.03.

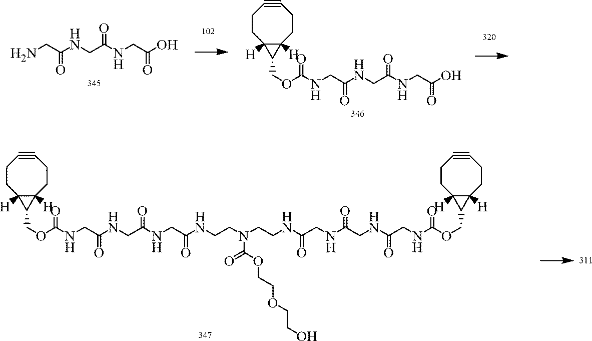

### Example 81. Synthesis of 346

To an Eppendorf vial containing 102 (54.7 mg, 1.00 Eq, 173 µmol) and 345 (triglycine, 28.8 mg, 0.878 equiv., 152 µmol) was added dry DMF (250 µL) and triethylamine (52.7 mg, 72.5 µL, 3 Eq, 520 µmol). The resulting yellow suspension was stirred at rt for 21 hours, followed by the addition of 50 µL H₂O to the RM. The reaction mixture was stirred at rt for another day upon which additional H₂O (200 µL) was added and the reaction mixture was stirred at rt for another 3 days. Next, MeCN (circa 0.5 mL) and additional Et₃N (circa 10 drops) were added and the resulting suspension was stirred for 1 hour at rt before conc. in vacuo. The yellow residue was taken up in DMF (600 µL) and the resulting yellow suspension was filtered over a membrane filter. The membrane-filter was washed with 200 µL additional DMF and the combined filtrates was purified directly via RP HPLC (Column Xbridge prep C18 5 µm OBD, 30x100 mm, 30% → 90% MeCN (1% AcOH) in water (1% AcOH). The desired product 346 was obtained as a brown oil (41.5 mg, 114 µmol, 66% yield). LCMS (ESI+) calculated for C₁₇H₂₄N₃O₆⁺ (M+H⁺) 366.17. found 366.27.

### Example 82. Synthesis of 347

To a solution of 346 (21.6 mg, 0.056 mmol) in anhydrous DMF (0.3 mL) were added DIPEA (30 µL, 0.171 mmol) and HATU (21.6 mg, 0.056 mmol). After stirring at room temperature for 10 min, 320 (7.37 mg, 0.031 mmol) dissolved in DCM (310 µL) was added. After stirring at room temperature for 24 h, the mixture was purified by RP HPLC (Column Xbridge prep C18 5 µm OBD, 30x100 mm, 30% → 100% MeCN in H₂O (both containing 1% AcOH)). The product 347 was obtained as an off- white oil (5.2 mg, 0.005 mmol, 20%). LCMS (ESI+) calculated for C₄₃H₆₄N₉O₁₄⁺ (M+H⁺) 931.02 found 931.68.

### Example 83. Synthesis of 311 (LD13)

To a solution of 347 (5.2 mg, 0.0056 mmol) in anhydrous DMF (200 µL) were added bis(4-nitrophenyl) carbonate (1.9 mg, 0.006 mmol) and Et₃N (2.4 µL, 0.016 mmol). After stirring at room temperature for 27 h, a stock solution of vc-PABC-MMAE.TFA (Levena Bioscience) (66 µL, 6.6 mg, 0.0053 mmol) and Et₃N (2 µL, 0.014 mmol) were added. After stirring for another 17 h at room temperature, the crude mixture was diluted with DMF (250 µL) and purified by RP HPLC (Column Xbridge prep C18 5 µm OBD, 30x100 mm, 5% → 90% MeCN in H₂O (both containing 1% AcOH)). The product 311 was obtained as a clear oil (0.6 mg, 0.28 µmol, 5%). LCMS (ESI+) calculated for C₁₀₂H₁₅₆N₁₉O₂₇⁺ (M/2+H⁺) 1040.71 found 1040.85.

### Example 84. Synthesis of Compound 312 (LD11)

Compound 312 (LD11) was prepared according to the procedure described by Verkade et al., Antibodies 2018, 7, doi:10.3390/antib7010012, incorporated by reference.

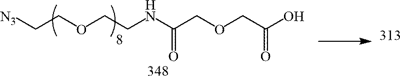

### Example 85. Synthesis of 313 (LD12)

To a vial containing 348 (2.7 mg, 1.1 Eq, 4.9 µmol) was added DMF (60 µL) and neat triethylamine (1.9 µL, 3 Eq, 13 µmol). Next, a solution of HBTU in dry DMF (2.0 mg, 11 µL, 472 mmolar, 1.2 Eq, 5.3 µmol) was added and the mixture was mixed. The reaction mixture was left at rt for 30 minutes, followed by the addition of va-PABC-MMAF.TFA salt (5.2 mg, 0.13 mL, 34.31 mmolar, 1 Eq, 4.4 µmol). The resulting mixture was mixed and left at rt for 110 minutes and was then purified directly via RP HPLC (Column Xbridge prep C18 5 µm OBD, 30x100 mm, 30% → 90% MeCN (1% AcOH) in water (1% AcOH). The desired product 313 was obtained as a colorless oil (1.8 mg, 1.1 µmol, 26% yield). LCMS (ESI+) calculated for C₇₇H₁₂₇N₁₂O₂₃⁺ (M+H⁺) 1587.91. found 1588.05.

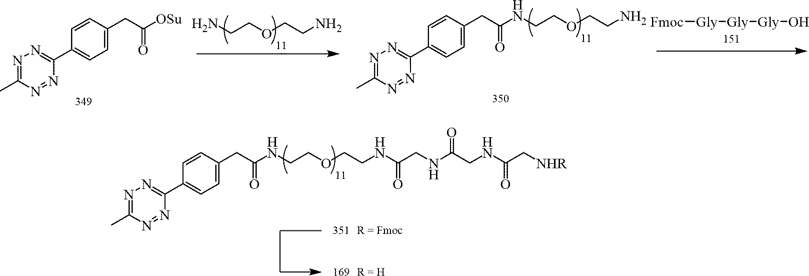

### Example 86. Synthesis of 350

To a solution of methyltetrazine-NHS ester 349 (19 mg, 0.057 mmol) in DCM (400 µL) was added amino-PEG₁₁-amine (47 mg, 0.086 mmol) dissolved in DCM (800 µL). After stirring at room temperature for 20 min, the mixture was concentrated in vacuo and purified by flash column chromatography over silicagel (0 → 50% MeOH (0.7 M NH₃) in DCM) which gave the desired compound 350 as a pink oil (17 mg, 0.022 mmol, 39%). LCMS (ESI+) calculated for C₃₅H₆₁N₆O₁₂⁺ (M+H⁺) 757.89 found 757.46.

### Example 87. Synthesis of 351

To a stirred solution of 151 (Fmoc—Gly—Gly—Gly—OH, 10 mg, 0.022 mmol) in anhydrous DMF (500 µL) were added DIPEA (11 µL, 0.067 mmol) and HATU (8.5 mg, 0.022 mmol). After 10 min, 350 (17 mg, 0.022 mmol) dissolved in anhydrous DMF (500 µL) was added. After stirring at room temperature for 18.5 h, the mixture was concentrated in vacuo and purified by flash column chromatography over silicagel (0 → 17% MeOH in DCM) which gave the desired compound 351 as a pink oil (26 mg, 0.022 mmol, quant.). LCMS (ESI+) calculated for C₅₆H₈₃N₁₀O₁₇⁺ (M+NH₄⁺) 1168.32 found 1168.67

### Example 88. Synthesis of 169

To a solution of 351 (26 mg, 0.022 mmol) in anhydrous DMF (500 µL) was added diethylamine (12 µL, 0.11 mmol). After stirring at room temperature for 1.5 h, the crude mixture was purified by RP HPLC (Column Xbridge prep C18 5 µm OBD, 30x100 mm, 5% → 90% MeCN in H₂O (both containing 1% acetic acid)). The product 169 was obtained as a clear pink oil (10.9 mg, 0.011 mmol, 53%). LCMS (ESI+) calculated for C₄₁H₇₀N₉O₁₅⁺ (M+H⁺) 929.05 found 929.61.

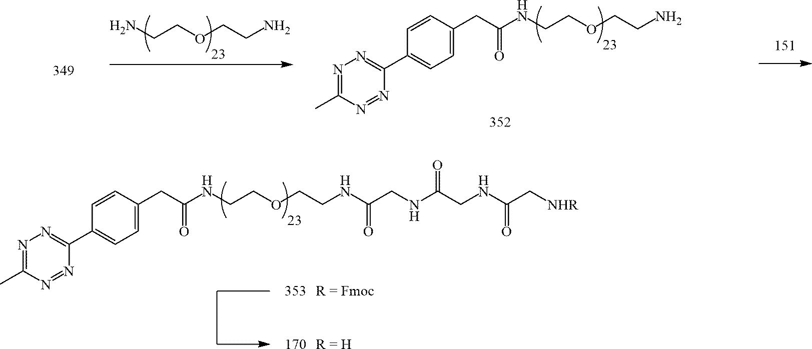

### Example 89. Synthesis of 352

To a solution of 349 (methyltetrazine-NHS ester, 10.3 mg, 0.031 mmol) in DCM (200 µL) was added amino-PEG₂₃-amine (50 mg, 0.046 mmol) dissolved in DCM (200 µL). After stirring at room temperature for 50 min, the mixture was concentrated in vacuo and purified by flash column chromatography over silicagel (0 → 60% MeOH (0.7 M NH₃) in DCM) which gave the desired compound 352 as a pink oil (17.7 mg, 0.013 mmol, 44%). LCMS (ESI+) calculated for C₅₉H₁₀₉N₆O₂₄⁺ (M+H⁺) 1286.52 found 1286.72.

### Example 90. Synthesis of 353

To a stirred solution of 151 (5.7 mg, 0.013 mmol) in anhydrous DMF (500 µL) were added DIPEA (7 µL, 0.04 mmol) and HATU (5.3 mg, 0.013 mmol). After 10 min, 352 (17.7 mg, 0.013 mmol) dissolved in anhydrous DMF (500 µL) was added. After stirring at room temperature for 6 h, the mixture was concentrated in vacuo and purified by flash column chromatography over silicagel (0 → 18% MeOH in DCM) which gave the desired compound 353 as a pink oil (21 mg, 0.012 mmol, 91%). LCMS (ESI+) calculated for C₈₀H₁₃₁N₁₀O₂₉⁺ (M/2+NH₄⁺) 857.45 found 857.08

### Example 91. Synthesis of 170

To a solution of 353 (21 mg, 0.012 mmol) in anhydrous DMF (500 µL) was added diethylamine (6.7 µL, 0.06 mmol). After stirring at room temperature for 4 h, the crude mixture was purified by RP HPLC (Column Xbridge prep C18 5 µm OBD, 30x100 mm, 5% → 90% MeCN in H₂O (both containing 1% acetic acid)). The product 170 was obtained as a pink oil (11.6 mg, 0.008 mmol, 66%). LCMS (ESI+) calculated for C₆₅H₁₁₈N₉O₂₇⁺ (M+H⁺) 1457.68 found 1457.92.

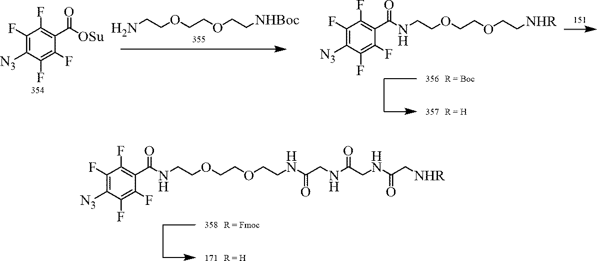

### Example 92. Synthesis of 356

To a solution of 354 (tetrafluorophenylazide-NHS ester, 40 mg, 0.12 mmol) in DCM (1 mL) were added 355 (Boc-NH-PEG₂-NH₂, 33 mg, 0.13 mmol) and Et₃N (50 µL, 0.36 mmol). After stirring in the dark at room temperature for 30 min, the mixture was concentrated in vacuo and purified by flash column chromatography over silicagel (0 → 7% MeOH in DCM) which gave the desired compound 356 as a clear oil (47 mg, 0.10 mmol, 84%). LCMS (ESI+) calculated for C₁₈H₂₄F₄N₅O₅⁺ (M+H⁺) 466.41 found 466.23.

### Example 93. Synthesis of 357

To a solution of 356 (47 mg, 0.10 mmol) in DCM (2 mL) was added 4.0 M HCl in dioxane (300 µL). After stirring in the dark at room temperature for 17.5 h, the mixture was concentrated and 357 was obtained as a white solid in quantitative yield (36 mg, 0.10 mmol). LCMS (ESI+) calculated for C₁₃H₁₆F₄N₅O₃⁺ (M+H⁺) 366.29 found 366.20.

### Example 94. Synthesis of 358

To a stirred solution of 151 (Fmoc—Gly—Gly—Gly—OH, 42 mg, 0.10 mmol) in anhydrous DMF (600 µL) were added DIPEA (50 µL, 0.30 mmol) and HATU (39 mg, 0.10 mmol). After 15 min in the dark, 357 (36 mg, 0.10 mmol) dissolved in anhydrous DMF (500 µL) was added. After stirring in the dark at room temperature for 41 h, the mixture was concentrated in vacuo and purified by flash column chromatography over silicagel (0 → 20% MeOH in DCM) which gave the desired compound 358 as a clear oil (36 mg, 0.047 mmol, 47%). LCMS (ESI+) calculated for C₃₄H₃₅F₄N₈O₈⁺ (M+H⁺) 759.68 found 759.38.

### Example 95. Synthesis of 171

To a solution of 358 (36 mg, 0.047 mmol) in anhydrous DMF (750 µL) was added diethylamine (24 µL, 0.24 mmol). After stirring in the dark at room temperature for 55 min, the crude mixture was purified by RP HPLC (Column Xbridge prep C18 5 µm OBD, 30x100 mm, 5% → 90% MeCN in H₂O (both containing 1% acetic acid)). The product 171 was obtained as a clear oil (18.7 mg, 0.034 mmol, 74%). LCMS (ESI+) calculated for C₁₉H₂₅F₄N₈O₆⁺ (M+H⁺) 537.45 found 537.29.

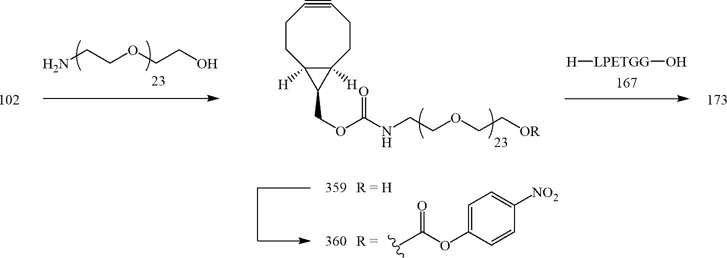

### Example 96. Synthesis of 359

To a solution of 102 (56 mg, 0.17 mmol) in DCM (8 mL) were added amino-PEG₂₄-alcohol (214 mg, 0.199 mmol) and Et₃N (80 µL, 0.53 mmol). After stirring at room temperature for 20 h, solvent was reduced in vacuo and the residue was purified by flash silica gel column chromatography (2 → 30% MeOH in DCM) which gave the desired compound 359 as a yellow oil in 95% yield (210 mg, 0.168 mmol). LCMS (ESI+) calculated for C₅₉H₁₁₁NO₂₆Na⁺ (M+Na⁺) 1273.50 found 1273.07.

### Example 97. Synthesis of 360

To a solution of 359 (170 mg, 0.136 mmol) and 4-nitrophenyl chloroformate (44 mg, 0.22 mmol) in DCM (7 mL) was added Et₃N (63 µL, 0.40 mmol). After stirring at room temperature for 41 h, solvent was reduced and the residue was purified by flash silica gel column chromatography (0 → 10% MeOH in DCM) which gave the desired compound 360 as a clear oil in 67% yield (129 mg, 0.091 mmol). LCMS (ESI+) calculated for C₆₆H₁₁₄N₂O₃₀Na⁺ (M+Na+) 1438.59 found 1438.13.

### Example 98. Synthesis of 173

To a solution of 360 (16 mg, 0.011 mmol) in anhydrous DMF (800 µL) were added 167 (peptide H-LPETGG-OH, 6.5 mg, 0.011 mmol) and Et₃N (5 µL, 0.04 mmol). After stirring at room temperature for 95 h, the crude mixture was purified by RP HPLC (Column Xbridge prep C18 5 µm OBD, 30x100 mm, 5% → 90% MeCN in H₂O (both containing 1% acetic acid)). The product 173 was obtained as a clear oil (12.6 mg, 0.0068 mmol, 62%). LCMS (ESI+) calculated for C₈₄H₁₅₃N₈O₃₇⁺ (M/2+NH₄⁺) 942.55 found 924.26.

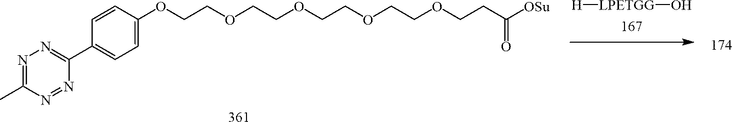

### Example 99. Synthesis of 174

To a solution of 361 (methyltetrazine-PEG₅-NHS ester, 6.1 mg, 0.011 mmol) in anhydrous DMF (230 µL) were added peptide H-LPETGG-OH (6.5 mg, 0.011 mmol) and Et₃N (4 µL, 0.028 mmol). After stirring at room temperature for 22 h, the crude mixture was purified by RP HPLC (Column Xbridge prep C18 5 µm OBD, 30x100 mm, 5% → 90% MeCN in H₂O (both containing 1% acetic acid)). The product 174 was obtained as a clear pink oil (9.9 mg, 0.01 mmol, 91%). LCMS (ESI+) calculated for C₄₄H₇₀N₁₁O₁₆⁺ (M+NH₄⁺) 1009.09 found 1009.61.

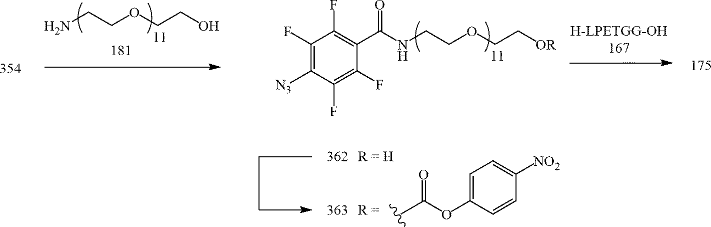

### Example 100. Synthesis of 362

To a solution of 354 (31 mg, 0.093 mmol) in DCM (1 mL) were added 181 (56 mg, 0.10 mmol) and Et₃N (40 µL, 0.28 mmol). After stirring in the dark at room temperature for 25 min, the mixture was concentrated in vacuo and purified by flash column chromatography over silicagel (0 → 15% MeOH in DCM) which gave the desired compound 362 as a clear oil (55 mg, 0.072 mmol, 77%). LCMS (ESI+) calculated for C₃₁H₅₁F₄N₄O₁₃⁺ (M+H⁺) 763.75 found 763.08.

### Example 101. Synthesis of 363

To a solution of 362 (55 mg, 0.072 mmol) in DCM (2 mL) were added 4-nitrophenyl chloroformate (13 mg, 0.064 mmol) and Et₃N (30 µL, 0.21 mmol). After stirring in the dark at room temperature for 21 h, the mixture was concentrated in vacuo and purified by RP HPLC (Column Xbridge prep C18 5 µm OBD, 30x100 mm, 5% → 90% MeCN (1% AcOH) in water (1% AcOH). The product 363 was obtained as a yellow oil (13.3 mg, 0.014 mmol, 20%). LCMS (ESI+) calculated for C₃₈H₅₄F₄N₅O₁₇⁺ (M+H⁺) 928.85 found 928.57.

### Example 102. Synthesis of 175

To a solution of 363 (13.3 mg, 0.014 mmol) in anhydrous DMF (300 µL) were added 167 (peptide H-LPETGG-OH, 8.2 mg, 0.014 mmol) and Et₃N (6 µL, 0.043 mmol). After 26 h in the dark, the crude mixture was purified by RP HPLC (Column Xbridge prep C18 5 µm OBD, 30x100 mm, 5% → 90% MeCN in H₂O (both containing 1% acetic acid)). The product 175 was obtained as a clear oil (11.4 mg, 0.0084 mmol, 59%). LCMS (ESI+) calculated for C₅₆H₈₉F₄N₁₀O₂₄⁺ (M+H⁺) 1362.35 found 1362.81.

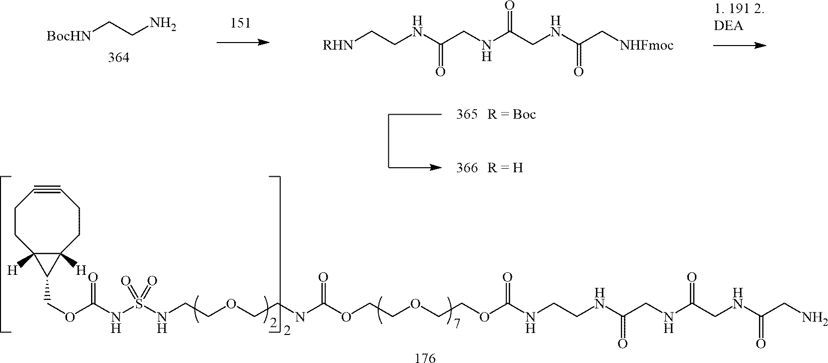

### Example 103. Synthesis of 365

To a stirred solution of 151 (Fmoc—Gly—Gly—Gly—OH, 20 mg, 0.049 mmol) in anhydrous DMF (350 µL) were added DIPEA (25 µL, 0.15 mmol) and HATU (18 mg, 0.049 mmol). After 10 min, compound 364 (N—Boc—ethylenediamine, 7.8 mg, 0.049 mmol) dissolved in anhydrous was added. After stirring at room temperature for 45 min, the mixture was concentrated in vacuo and purified by flash column chromatography over silicagel (0 → 30% MeOH in DCM) which gave the desired compound 365 as a clear oil (12.4 mg, 0.022 mmol, 46%). LCMS (ESI+) calculated for C₂₈H₃₆N₅O₇⁺ (M+H⁺) 554.61 found 554.46.

### Example 104. Synthesis of 366

To a stirred solution of 365 (12.4 mg, 0.022 mmol) in DCM (0.7 mL) was added 4.0 M HCl in dioxane (400 µL). After stirring at room temperature for 1 h, the mixture was concentrated and 366 was obtained as a white solid (11 mg, 0.022 mmol, quant.). LCMS (ESI+) calculated for C₂₃H₂₈N₅O7⁺ (M+H⁺) 545.50 found 454.33.

### Example 105. Synthesis of 176

To a solution of 191 (8 mg, 0.0059 mmol) in anhydrous DMF (300 µL) were added Et₃N (2.5 µL, 0.017 mmol) and stock of 366 in anhydrous DMF (110 µL, 3.0 mg, 0.0059 mmol). After stirring at room temperature for 18 h, diethylamine (2 uL) was added. After an additional of 2 h, the mixture was purified by RP HPLC (Column Xbridge prep C18 5 µm OBD, 30x100 mm, 5% → 90% MeCN in H₂O (both containing 1% acetic acid)). The product 176 was obtained as a clear oil (1.3 mg, 0.0009 mmol, 15%). LCMS (ESI+) calculated for C₆₀H₁₀₃N₁₀O₂₆S₂⁺ (M+H⁺) 1444.64 found 1444.75.

### Example 106. Anti-4-1BB PF31

Anti1 BB scFv was designed with a C-terminal sortase A recognition sequence followed by a His tag (amino acid sequence being identified by SEQ ID NO: 4). Anti1BB scFv was transiently expressed in HEK293 cells followed by IMAC purification by Absolute Antibody Ltd (Oxford, United Kingdom). Mass spectral analysis showed one major product (observed mass 28013 Da, expected mass 28018 Da).

### Example 107. Cloning of SYR-(G₄S)₃-IL15 (PF18) Into pET32a Expression Vector

The SYR-(G₄S)₃-IL15 (PF18) (amino acid sequence being identified by SEQ ID NO: 5) was designed with an N-terminal (M)SYR sequence, where the methionine will be cleaved after expression leaving an N-terminal serine, and a flexible (G4S)₃ spacer between the SYR sequence and IL15. The codon-optimized DNA sequence was inserted into a pET32A expression vector between Ndel and Xhol, thereby removing the sequence encoding the thioredoxin fusion protein, and was obtained from Genscript, Piscataway, USA.

### Example 108. E. Coli Expression of SYR-(G₄S)₃-IL15 (PF18) and Inclusion Body Isolation

Expression of SYR-(G₄S)₃-IL15 (PF18) starts with the transformation of the plasmid (pET32a-SYR-(G₄S)₃-IL15) into BL21 cells (Novagen). Transformed cells were plated on LB-agar with ampicillin and incubated overnight at 37° C. A single colony was picked and used to inoculate 50 mL of TB medium + ampicillin followed by incubated overnight at 37° C. Next, the overnight culture was used to inoculation 1000 mL TB medium + ampicillin. The culture was incubated at 37° C. at 160 RPM and, when OD600 reached 1.5, induced with 1 mM IPTG (1 mL of 1 M stock solution). After >16 hour induction at 37° C. at 160 RPM, the culture was pelleted by centrifugation (5000 xg - 5 min). The cell pellet gained from 1000 mL culture was lysed in 60 mL BugBuster™ with 1500 units of Benzonase and incubated on roller bank for 30 min at room temperature. After lysis the insoluble fraction was separated from the soluble fraction by centrifugation (15 minutes, 15000 x g). Half of the insoluble fraction was dissolved in 30 mL BugBuster™ with lysozyme (final concentration: 200 µg/mL) and incubated on the roller bank for 10 min. Next the solution was diluted with 6 volumes of 1:10 diluted BugBuster™ and centrifuged 15 min, 15000 x g . The pellet was resuspended in 200 mL of 1:10 diluted BugBuster™ by using the homogenizer and centrifuged at 10 min, 12000 x g. The last step was repeated 3 times.

### Example 109. Refolding of SYR-(G₄S)₃-IL15 (PF18) from Isolated Inclusion Bodies

The purified inclusion bodies containing SYR-(G₄S)₃-IL15 (PF18), were dissolved and denatured in 30 mL 5 M guanidine with 40 mM Cysteamine and 20 mM Tris pH 8.0. The suspension was centrifuged at 16.000 x g for 5 min to pellet the remaining cell debris. The supernatant was diluted to 1 mg/mL with 5 M guanidine with 40 mM Cysteamine and 20 mM Tris pH 8.0, and incubated for 2 hours at RT on a rollerbank. The 1 mg/mL solution is added dropwise to 10 volumes of refolding buffer (50 mM Tris, 10.53 mM NaCl, 0.44 mM KCl, 2.2 mM MgCl₂, 2.2 mM CaCl₂, 0.055% PEG-4000, 0.55 M L-arginine, 4 mM cysteamine, 4 mM cystamine, at pH 8.0) in a cold room at 4° C., stirring required. Leave solution at least 24 hours at 4° C. Dialyze the solution to 10 mM NaCl and 20 mM Tris pH 8.0, 1x overnight and 2x 4 hours, using a Spectrum™ Spectra/Por™ 3 RC Dialysis Membrane Tubing 3500 Dalton MWCO. Refolded SYR-(G₄S)₃-IL15 (PF18) was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 M NaCl, pH 8.0) on a gradient of 30 mL from buffer A to buffer B. Mass spectrometry analysis showed a weight of 14122 Da (expected mass: 14122 Da) corresponding to PF18. The purified SYR-(G₄S)₃-IL15 (PF18) was buffer exchanged to PBS using HiPrep™ 26/10 Desalting column (Cytiva) on a AKTA Purifier-10 (GE Healthcare).

### Example 110. Cloning of SYR-(G₄S)₃-IL15Ra-Linker-IL15 (PF26) Into pET32a Expression Vector

The SYR-(G₄S)₃-IL15Ra-linker-IL15 (PF26) (amino acid sequence being identified by SEQ ID NO: 6) was designed with an N-terminal (M)SYR sequence, where the methionine will be cleaved after expression leaving an N-terminal serine, and a flexible (G₄S)₃ spacer between the SYR sequence and IL15Ra-linker-IL15. The codon-optimized DNA sequence was inserted into a pET32A expression vector between Ndel and Xhol, thereby removing the sequence encoding the thioredoxin fusion protein, and was obtained from Genscript, Piscataway, USA.

### Example 111. E. Coli Expression of SYR-(G₄S)₃-IL15Ra-Linker-IL15 (PF26) and Inclusion Body Isolation

Expression of SYR-(G₄S)₃-IL15Ra-linker-IL15 (PF26) starts with the transformation of the plasmid (pET32a-SYR-(G₄S)₃-IL15Ra-linker-IL15) into BL21 cells (Novagen). Next step was the inoculation of 1000 mL culture (TB medium + ampicillin) with BL21 cells. When OD600 reached 1.5, cultures were induced with 1 mM IPTG (1 mL of 1 M stock solution). After >16 hour induction at 37° C. at 160 RPM, the culture was pelleted by centrifugation (5000 xg - 5 min). The cell pellet gained from 1000 mL culture was lysed in 60 mL BugBuster™ with 1500 units of Benzonase and incubated on roller bank for 30 min at room temperature. After lysis the insoluble fraction was separated from the soluble fraction by centrifugation (15 minutes, 15000 x g). Half of the insoluble fraction was dissolved in 30 mL BugBuster™ with lysozyme (final concentration: 200 µg/mL) and incubated on the roller bank for 10 min. Next the solution was diluted with 6 volumes of 1:10 diluted BugBuster™ and centrifuged 15 min, 15000 x g. The pellet was resuspended in 200 mL of 1:10 diluted BugBuster™ by using the homogenizer and centrifuged at 10 min, 12000 x g. The last step was repeated 3 times.

### Example 112. Refolding of SYR-(G₄S)₃-IL15Ra-linker-IL15 (PF26) From Isolated Inclusion Bodies

The purified inclusion bodies containing SYR-(G₄S)₃-IL15Ra-linker-IL15 (PF26), were dissolved and denatured in 30 mL 5 M guanidine with 40 mM Cysteamine and 20 mM Tris pH 8.0. The suspension was centrifuged at 16.000 x g for 5 min to pellet the remaining cell debris. The supernatant was diluted to 1 mg/mL with 5 M guanidine with 40 mM Cysteamine and 20 mM Tris pH 8.0, and incubated for 2 hours at RT on a rollerbank. The 1 mg/mL solution is added dropwise to 10 volumes of refolding buffer (50 mM Tris, 10.53 mM NaCl, 0.44 mM KCI, 2.2 mM MgCl₂, 2.2 mM CaCl₂, 0.055% PEG-4000, 0.55 M L-arginine, 4 mM cysteamine, 4 mM cystamine, at pH 8.0) in a cold room at 4° C., stirring required. Leave solution at least 24 hours at 4° C. Dialyze the solution to 10 mM NaCl and 20 mM Tris pH 8.0, 1x overnight and 2x 4 hours using a Spectrum™ Spectra/Por™ 3 RC Dialysis Membrane Tubing 3500 Dalton MWCO. Refolded SYR-(G₄S)₃-IL15Ra-linker-IL15 (PF26) was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 M NaCl, pH 8.0) on a gradient of 30 mL from buffer A to buffer B. Mass spectrometry analysis showed a weight of 24146 Da (expected mass: 24146 Da) corresponding to PF26. The purified SYR-(G₄S)₃-IL15Ra-linker-IL15 (PF26) was buffer exchanged to PBS using HiPrep™ 26/10 Desalting column from cytiva on a AKTA Purifier-10 (GE Healthcare).

### Example 113 Humanized OKT3 200

Humanized OKT3 (hOKT3) with C-terminal sortase A recognition sequence (C-terminal tag being identified by SEQ ID NO: 1) was obtained from Absolute Antibody Ltd (Oxford, United Kingdom). Mass spectral analysis showed one major product (observed mass 28836 Da).

### Example 114. C-terminal Sortagging of Compound GGG-PEG₂-BCN (157) to hOKT3 200 Using Sortase A to Obtain hOKT3-PEG₂-BCN 201

A bioconjugate according to the invention was prepared by C-terminal sortagging using sortase A (identified by SEQ ID NO: 2). To a solution of hOKT3 200 (500 µL, 500 µg, 35 µM in PBS pH 7.4) was added sortase A (58 µL, 384 µg, 302 µM in TBS pH 7.5 + 10% glycerol), GGG-PEG₂-BCN (157, 28 µL, 50 mM in DMSO), CaCl₂ (69 µL, 100 mM in MQ) and TBS pH 7.5 (39 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27829 Da), corresponding to 201. The sample was dialyzed against PBS pH 7.4 and concentrated by spinfiltration (Amicon Ultra-0.5, Ultracel-10 Membrane, Millipore) to obtain hOKT3-PEG₂-BCN 201 (60 µL, 169 µg, 101 µM in PBS pH 7.4).

### Example 115. C-terminal Sortagging of Compound GGG-PEG₂-BCN (157) to hOKT3 200 Using sortase A Pentamutant to Obtain hOKT3-PEG₂-BCN 201

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₂-BCN (157, 2 µL, 20 mM in DMSO:MQ=2:3), CaCl₂ (2 µL, 100 mM in MQ) and TBS pH 7.5 (1.2 µL). The reaction was incubated at 37° C. overnight. Mass spectral analysis showed one major product (observed mass 27829 Da), corresponding to hOKT3-PEG₂-BCN 201.

### Example 116. C-terminal Sortagging of Compound GGG-PEG₁₁-BCN (161) to hOKT3 200 Using Sortase A to Obtain hOKT3-PEG₁₁-BCN 202

A bioconjugate according to the invention was prepared by C-terminal sortagging using sortase A (identified by SEQ ID NO: 2). To a solution of hOKT3 200 (14.3 µL, 14 µg, 35 µM in PBS pH 7.4) was added sortase A (0.9 µL, 12 µg, 582 µM in TBS pH 7.5 + 10% glycerol), GGG-PEG₁₁-BCN (161, 2 µL, 20 mM in MQ), CaCl₂ (2 µL, 100 mM in MQ) and TBS pH 7.5 (0.9 µL). The reaction was incubated at 37° C. overnight. Mass spectral analysis showed one major product (observed mass 21951 Da, approximately 85%), corresponding to sortase A, a minor product (observed masses 28227 Da, approximately 5%), corresponding to hOKT3-PEG₁₁-BCN 202, and two other minor products (observed masses 28051 Da and 28325 Da, each approximately 5%).

### Example 117. C-terminal Sortagging of Compound GGG-PEG₁₁-BCN (161) to hOKT3 200 Using Sortase A Pentamutant to Obtain hOKT3-PEG₁₁-BCN 202

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₁₁-BCN (161, 2 µL, 20 mM in MQ), CaCl₂ (2 µL, 100 mM in MQ) and TBS pH 7.5 (1.2 µL). The reaction was incubated at 37° C. overnight. Mass spectral analysis showed one major product (observed mass 28225 Da, approximately 60%), corresponding to hOKT3-PEG₁₁-BCN 202, and one minor product (observed mass 28326 Da, approximately 40%).

### Example 118. C-terminal Sortagging of Compound GGG-PEG₂₃-BCN (163) to hOKT3 200 Using sortase A to Obtain hOKT3-PEG₂₃-BCN 203

A bioconjugate according to the invention was prepared by C-terminal sortagging using sortase A (identified by SEQ ID NO: 2). To a solution of hOKT3 200 (14.3 µL, 14 µg, 35 µM in PBS pH 7.4) was added sortase A (0.9 µL, 12 µg, 582 µM in TBS pH 7.5 + 10% glycerol), GGG-PEG₂₃-BCN (163, 2 µL, 20 mM in MQ), CaCl₂ (2 µL, 100 mM in MQ) and TBS pH 7.5 (0.9 µL). The reaction was incubated at 37° C. overnight. Mass spectral analysis showed one major product (observed mass 21951 Da, approximately 70%), corresponding to sortase A, and one minor product (observed mass 28755 Da, approximately 30%), corresponding to hOKT3-PEG₂₃-BCN 203.

### Example 119. C-terminal Sortagging of Compound GGG-PEG₂₃-BCN (163) to hOKT3 200 Using Sortase A Pentamutant to Obtain hOKT3-PEG₂₃-BCN 203

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCI, 400 mM imidazole and 20% glycerol), GGG-PEG₂₃-BCN (163, 2 µL, 20 mM in MQ), CaCl₂ (2 µL, 100 mM in MQ) and TBS pH 7.5 (1.2 µL). The reaction was incubated at 37° C. overnight. Mass spectral analysis showed one major product (observed mass 28754 Da), corresponding to hOKT3-PEG₂₃-BCN 203.

### Example 120. C-Terminal Sortagging of Compound GGG-PEG₄-tetrazine (154) to hOKT3 200 Using Sortase A to Obtain hOKT3-PEG₄-Tetrazine 204

A bioconjugate according to the invention was prepared by C-terminal sortagging using sortase A (identified by SEQ ID NO: 2). To a solution of hOKT3 200 (500 µL, 500 µg, 35 µM in PBS pH 7.4) was added sortase A (58 µL, 384 µg, 302 µM in TBS pH 7.5 + 10% glycerol), GGG-PEG₄-tetrazine (154, 35 µL, 40 mM in MQ), CaCl₂ (69 µL, 100 mM in MQ) and TBS pH 7.5 (32 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27868 Da), corresponding to 104. The sample was dialyzed against PBS pH 7.4 and concentrated by spinfiltration (Amicon Ultra-0.5, Ultracel-10 Membrane, Millipore) to obtain hOKT3-PEG₄-tetrazine 204 (70 µL, 277 µg, 143 µM in PBS pH 7.4).

### Example 121. C-Terminal Sortagging of Compound GGG-PEG₄-tetrazine (154) to hOKT3 200 Using Sortase A Pentamutant to Obtain hOKT3-PEG₄-Tetrazine 204

A bioconjugate according to the invention was prepared by C-terminal sortagging using sortase A pentamutant (BPS Bioscience, catalog number 71046). To a solution of hOKT3 200 (14.3 µL, 14 µg, 35 µM in PBS pH 7.4) was added sortase A pentamutant (0.5 µL, 1 µg, 92 µM in 40 mM Tris pH8.0, 110 mM NaCl, 2.2 mM KCl, 400 mM imidazole and 20% glycerol), GGG-PEG₄-tetrazine (154, 2 µL, 20 mM in MQ), CaCl₂ (2 µL, 100 mM in MQ) and TBS pH 7.5 (1.2 µL). The reaction was incubated at 37° C. overnight. Mass spectral analysis showed one major product (observed mass 27868 Da), corresponding to hOKT3-PEG₄-tetrazine 204.

### Example 122. C-Terminal Sortagging of GGG-PEG₁₁-Tetrazine (169) to hOKT3 200 With Sortase A to Obtain hOKT3-PEG₁₁-Tetrazine PF01

A bioconjugate according to the invention was prepared by C-terminal sortagging with sortase A (identified by SEQ ID NO: 2). To a solution of hOKT3 200 (1908 µL, 5 mg, 91 µM in PBS pH 7.4) was added sortase A (81 µL, 948 µg, 533 µM in TBS pH 7.5 + 10% glycerol), GGG-PEG₁₁-tetrazine (169, 347 µL, 20 mM in MQ), CaCl₂ (347 µL, 100 mM in MQ) and TBS pH 7.5 (789 µL). The reaction was incubated at 37° C. overnight. Mass spectral analysis showed one major product (observed mass 28258 Da), corresponding to hOKT3-PEG₁₁-tetrazine PF01. The reaction was purified on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and buffer exchanged to PBS pH 6.5 using a HiPrep 26/10 desalting column (GE Healthcare). Addition dialysis was performed to PBS pH 6.5 for 3 days at 4° C. to remove residual 169.

### Example 123. C-terminal Sortagging of GGG-PEG₂₃-Tetrazine (170) to hOKT3 200 With Sortase A to Obtain hOKT3-PEG₂₃-Tetrazine PF02

A bioconjugate according to the invention was prepared by C-terminal sortagging with sortase A (identified by SEQ ID NO: 2). To a solution of hOKT3 200 (1908 µL, 5 mg, 91 µM in PBS pH 7.4) was added sortase A (81 µL, 948 µg, 533 µM in TBS pH 7.5 + 10% glycerol), GGG-PEG₂₃-tetrazine (170, 347 µL, 20 mM in MQ), CaCl₂ (347 µL, 100 mM in MQ) and TBS pH 7.5 (789 µL). The reaction was incubated at 37° C. overnight. Mass spectral analysis showed one major product (observed mass 28787 Da), corresponding to hOKT3-PEG₂₃-tetrazine PF02. The reaction was purified on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was dialyzed to PBS pH 6.5 followed by purification on a Superdex75 10/300 GL column (GE Healthcare) on an AKTA Purifier-10 (GE Healthcare) using PBS pH 6.5 as mobile phase.

### Example 124. C-terminal Sortagging of GGG-PEG₂-Arylazide (171) to hOKT3 200 With Sortase A to Obtain hOKT3-PEG₂-Arylazide PF03

A bioconjugate according to the invention was prepared by C-terminal sortagging with sortase A (identified by SEQ ID NO: 2). To a solution of hOKT3 200 (2092 µL, 5 mg, 83 µM in PBS pH 7.4) was added sortase A (95 µL, 950 µg, 456 µM in TBS pH 7.5 + 10% glycerol), GGG-PEG₂-arylazide (171, 347 µL, 20 mM in MQ), CaCl₂ (347 µL, 100 mM in MQ) and TBS pH 7.5 (591 µL). The reaction was incubated at 37° C. overnight. Mass spectral analysis showed one major product (observed mass 27865 Da), corresponding to hOKT3-PEG₂-arylazide PF03. The reaction was purified on a His-trap excel 1 mL column (GE Healthcare) on an AKTA Purifier-10 (GE Healthcare). The column was equilibrated with buffer A (20 mM Tris, 200 mM NaCl, 20 mM Imidazole, pH 7.5) and the sample was loaded with 1 mL/min. The flowthrough purified on a Superdex75 10/300 GL column (GE Healthcare) on an AKTA Purifier-10 (GE Healthcare) using PBS pH 7.4 as mobile phase.

### Example 125. C-terminal Sortagging of GGG-PEG₁₁-Tetrazine (169) in Anti-4-1BB PF31 With Sortase A to Obtain Anti-4-1BB-PEG₁₁-Tetrazine PF08

To a solution containing protein PF31 (1151 µL, 93 µM in TBS pH 7.5) was added TBS pH 7.5 (512 µL), CaCl₂ (214 µL, 100 mM) and GGG-PEG₁₁-tetrazine (169, 220µL, 20 mM in MQ) and Sortase A (50 µL, 533 µM in TBS pH 7.5). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (Observed mass 27989 Da) corresponding to 4-1BB-tetrazine PF08.

### Example 126. C-terminal Sortagging of Compound GGG-PEG₂-Arylazide (171) Anti-4-1BB-PF31 With Sortase A to Obtain Anti-4-1BB PF09

A bioconjugate according to the invention was prepared by C-terminal sortagging with sortase A (identified by SEQ ID NO: 2). To a solution of anti-4-1BB-PF31 (665 µL, 2 mg, 107 µM in PBS pH 7.4) was added sortase A (100 µL, 1 mg, 357 µM in TBS pH 7.5 + 10% glycerol), GGG-PEG₂-arylazide (171, 140 µL, 20 mM in MQ), CaCl₂ (140 µL, 100 mM in MQ) and TBS pH 7.5 (355 µL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27592 Da) corresponding to anti-4-1BB-azide PF09.

### Example 127. N-Terminal Sortagging of Arylazide-PEG₁₁-LPETGG (175) in GGG-IL15Rα-IL15 (208) With Sortase A to Obtain Arylazide-PEG₁₁-GGG-IL15Rα-IL15 (PF13)

To a solution containing protein 208 (2000 µL, 140 µM in TBS pH 7.5) was added TBS pH 7.5 (2686 µL), CaCl₂ (559 µL, 100 mM) and 175 (83 µL, 50 mM in DMSO) and Sortase A (260 µL, 537 µM in TBS pH 7.5) and incubated 3 hours at 37° C. (shielded from light). After incubation, Sortase A was removed from the solution using Ni-NTA beads (500 µL Beads=1mL slurry). The solution was incubated ON at 4° C. with Ni-NTA beads on a roller bank, whereafter the solution was centrifuged (5 min, 7.000 xg). The supernatant, which contained the product PF13, was collected by separation of the supernatant from the pellet. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 24193 Da (expected mass: 24193 Da) corresponding to PF13.

### Example 128. N-Terminaloxime Ligation of BCN-PEG₁₂-Aminooxy (XL13) to SYR-(G₄S)₃-IL15Rα-IL15 (PF26) to Obtain BCN-PEG₁₂-SYR-(G₄S)₃-IL15Rα-IL15 (PF14)

Prior to labeling of PF26, the N-terminal Serine was oxidated using Sodium periodate. To a solution containing protein PF26 (700 µL, 70 µM in PBS pH 7.4) was added PBS pH 7.4 (286 µL), NaIO₄ (0.98 µL, 100 mM in MQ) and L-methionine (5 µL, 100 mM in MQ) and incubated 5 minutes at 4° C. Mass spectrometry analysis showed a weight of 24114 & 24130 Da corresponding to the expected masses of 24114 (aldehyde) and 24132 Da (hydrate). Using a PD-10 desalting column the excess NaIO₄ and L-methionine were removed. The oxidated PF26 was concentrated to a concentration of 50 µM using Amicon spin filter 0.5, MWCO 10 kDa (Merck-Millipore). To a solution containing oxidized PF26 (416 µL, 50 µM in PBS pH 7.4) was added, XL13 (41.6 µL, 50 mM in DMSO). After ON incubation at 37° C. the reaction mixture was purified using PD-10 desalting columns packed with Sephadex G-25 resin (Cytiva) and eluted using PBS. Mass spectrometry analysis showed a weight of 25024 Da (expected mass: 25042 Da) corresponding to PF14.

### Example 129. N-terminal BCN Functionalization of IL15Rα-IL15 PF26 to Obtain BCN-IL15Rα-IL15 PF15

To IL15Rα-IL15 PF26 (2.9 mg, 50 µM in PBS) was added 2 eq NaIO₄ (4.8 µL of 50 mM stock in PBS) and 10 eq L-Methionine (12.5 µL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the elute (2.6 mg, 50 µM in PBS) was added 160 eq N-methylhydroxylamine.HCl (340 µL of 50 mM stock in PBS) and 160 eq p-Anisidine (340 µL of 50 mM stock in PBS). The reaction mixture was incubated for 3 hours at 25° C. Mass spectral analysis showed one single peak (observed mass 24143 Da) corresponding to N-methyl-imine-oxide-IL15. The reaction mixture was purified using PD-10 desalting columns packed with Sephadex G-25 resin (Cytiva) and eluted using PBS. To the elute (2.47 mg, 50 µM in PBS) was added 25 eq Bis-BCN-PEG₁₁ (105) (51 µL, 50 mM in DMSO) and 150 µL DMF. The reaction was incubated overnight at room temperature. The reaction was purified using a Superdex75 10/300 column (Cytiva). Mass spectral analysis showed one major peak corresponding to BCN-IL15Rα-IL15 PF15 (observed mass 25041 Da).

### Example 130. N-Terminal Diazotransfer Reaction of IL15 PF18 to Obtain Azido-IL15 PF19

To IL15 PF18 (5 mg, 50 µM in 0.1 M TEA buffer pH 8.0) imidazole-1-sulfonylazide hydrochloride (708 µL, 50 mM in 50 mM NaOH) was added and incubated overnight at 37° C. The reaction was purified using a HiPrep™ 26/10 Desalting column (Cytiva). Mass spectral analysis showed one main peak (observed mass 14147 Da) corresponding to azido-IL15 PF19.

### Example 131. N-Terminal Incorporation of tetrazine-PEG₁₂-2PCA (XL10) in SYR-(G₄S)₃-IL15 (PF18) Using 2PCA to Obtain Tetrazine-PEG₁₂-SYR-(G₄S)₃-IL15 (PF21)

To SYR-(G₄S)₃-IL15 (PF18) (1052 µL, 50 µM in PBS) was added 20 eq. Tetrazine-PEG₁₂-2PCA (XL10) (112 µL of 50 mM stock in DMSO) and 4359 µL PBS. The reaction was incubated overnight at 37° C. Using spinfiltration (Amicon Ultra-0.5, Ultracel-10 Membrane, Millipore) the sample was concentrated <1 mL and loaded on to a Superdex 75 10/300 GL column (GE Healthcare) on an AKTA Purifier-10 (GE Healthcare) using PBS pH 7.4 as mobile phase and a flow of 0.5 mL/min. Mass spectral analysis showed a weight of 24121 Da corresponding to the start material SYR-(G₄S)₃-IL15 (PF18) (Expected mass: 14121 Da) and the a mass of 15093 Da corresponding to the product PF21 (Expected mass: 15094 Da).

### Example 132. Conjugation of Tri-BCN (150) to hOKT3-PEG₂-Arylazide PF03 to Obtain bis-BCN-hOKT3 PF22

To a solution of hOKT3-PEG₂-arylazide PF03 (87 µL, 1 mg, 411 µM in PBS pH 7.4) was added PBS pH 7.4 (559 µL), DMF (49 µL) and compound 150 (22 µL, 40 mM solution in DMF, 25 equiv.). The reaction was incubated overnight at RT. Mass spectral analysis showed one major product (observed mass 29171 Da), corresponding to bis-BCN-hOKT3 PF22. The reaction was purified on a Superdex75 10/300 GL column (GE Healthcare) on an AKTA Purifier-10 (GE Healthcare) using PBS pH 7.4 as mobile phase.

### Example 133. C-terminal Sortagging of GGG-bis-BCN 176 to hOKT3 200 With Sortase A to Obtain 5 bis-BCN-hOKT3 PF23

A bioconjugate according to the invention was prepared by C-terminal sortagging with sortase A (identified by SEQ ID NO: 2). To a solution of hOKT3 200 (272 µL, 0.7 mg, 83 µM in PBS pH 7.4) was added sortase A (25 µL, 250 µg, 456 µM in TBS pH 7.5 + 10% glycerol), GGG-bis-BCN (176, 45 µL, 20 mM in DMSO), CaCl₂ (45 µL, 100 mM in MQ) and TBS pH 7.5 (64 µL). The reaction was incubated at 37° C. overnight. Mass spectral analysis showed one major product (observed mass 28772 Da), corresponding to bis-BCN-hOKT3 PF23. The reaction was purified on a Superdex75 10/300 GL column (GE Healthcare) on an AKTA Purifier-10 (GE Healthcare) using PBS pH 7.4 as mobile phase.

### Example 134. N-Terminal Incorporation of tri-BCN (150) in N₃-SYR-(G₄S)₃-IL15 (PF19) Using Strain-Promoted Alkyne-Azide Cycloaddition to Obtain bis-BCN-SYR-(G₄S)₃-IL15 (PF29)

To N₃-IL15 PF19 (706 µL, 50 µM in PBS) was added 4 eq tri-BCN (150) (3.5 µL of 40 mM stock in DMF) and 67 µL DMF. The reaction was incubated o/n at RT. Mass spectral analysis confirmed the formation of bis-BCN-SYR-(G₄S)₃-IL15 PF29 (observed mass 15453 Da, expected mass 15453 Da). The reaction mixture was purified using PD-10 desalting columns packed with Sephadex G- 25 resin (Cytiva) and eluted using PBS. Additional washing was performed using spin-filtration (Amicon Ultra-0.5, Ultracel-10 Membrane, Millipore), 6x with 400 µL PBS, to remove remaining tri-BCN (150).

### Example 135. Enzymatic Deglycosylation of Trastuzumab With PNGase F

Trastuzumab (Herzuma) (20 mg, 12.5 mg/mL in PBS pH 7.4) was incubated with PNGase F (16 µL, 8000 units) at 37° C. Mass spectral analysis of a sample after IdeS treatment showed one major Fc/2 product (observed mass 23787 Da) corresponding to the expected product.

### Example 136. Enzymatic Deglycosylation of Rituximab With PNGase F

Rituximab (6 mg, 10 mg/mL in PBS pH 7.4) was incubated with PNGase F (6 µL, 3000 units) at 37° C. Mass spectral analysis of a sample after IdeS treatment showed one major Fc/2 product (observed mass 23754 Da) corresponding to the expected product.

### Example 137. MTGase-Catalyzed Incorpation of azido-PEG₃-amine Onto Deglycosylated Trastuzumab to give bis-azido-trastuzumab trast-v3

To a solution of deglycosylated trastuzumab (806 µL, 10 mg, 12.4 mg/mL in PBS pH 7.4) was added PBS pH 7.4 (3544 µL), azido-PEG₃-amine (commercially available from BroadPharm, 500 µL, 10 mM solution in MQ, 75 equiv. compared to IgG) and recombinant microbial transglutaminase (commercially available from Zedira, 150 µL, 15 U, 0.1 U/µL). The reaction was incubated overnight at 37° C. Mass spectral analysis of an IdeS-digested sample showed one major product (observed mass 23988 Da), corresponding to bis-azido-trastuzumab trast-v3. The reaction was purified using a protA column (5 mL, MabSelect Sure, GE Healthcare) on an AKTA Explorer-100 (GE Healthcare) followed by dialysis to PBS pH 7.4.

### Example 138. MTGase-catalyzed Incorpation of Azido-PEG₃-amine Onto Deglycosylated Rituximab to Give Bis-azido-Rituximab rit-v3

To a solution of deglycosylated rituximab (90 µL, 1.8 mg, 20.2 mg/mL in PBS pH 7.4) was added PBS pH 7.4 (693 µL), azido-PEG₃-amine (commercially available from BroadPharm, 90 µL, 10 mM solution in MQ, 75 equiv. compared to IgG) and recombinant microbial transglutaminase (commercially available from Zedira, 27 µL, 2.7 U, 0.1 U/µL). The reaction was incubated overnight at 37° C. Mass spectral analysis of an IdeS-digested sample showed one major product (observed mass 23956 Da), corresponding to bis-azido-rituximab rit-v3. The reaction was buffer exchange to PBS pH 7.4 using centrifugal filters (Amicon Ultra-0.5 mL MWCO 10 kDa, Merck Millipore).

### Example 139. Conjugation of Trastuzumab(6-N₃-GaINAc)₂ 205 With 201 to Obtain Conjugate 206

A bioconjugate according to the invention was prepared by conjugation of BCN-modified hOKT3 201 to azide-modified trastuzumab 205. To a solution of trastuzumab-(6-N₃-GaINAc)₂ prepared according to WO2016170186 (205, 2 µL, 75 µg, 250 µM in PBS pH 7.4) was added hOKT3-PEG₂-BCN 201 (9.9 µL, 28 µg, 101 µM in PBS pH 7.4). The reaction was incubated at rt overnight. Mass spectral analysis of the Fabricator™-digested sample showed two major products (observed masses 24368 Da and 52196 Da, each approximately 50%), corresponding to the azido-modified Fc/2-fragment and conjugate 206, respectively.

### Example 140. Cloning of His₆-SSGENLYFQ-GGG-IL15Rα-IL15 Into pET32a Expression Vector

The IL15Rα-IL15 fusion protein 207 was designed with an N-terminal His-tag (HHHHHH), TEV protease recognition sequence (SSGENLYFQ) and an N-terminal sortase A recognition sequence (GGG). A pET32A-vector containing a DNA sequence encoding His₆-SSGENLYFQ-GGG-IL15Rα-IL15 (SEQ ID NO: 3) between base pairs 158 and 692, thereby removing the thioredoxin coding sequence, was obtained from Genscript.

### Example 141. E. Coli Expression of His₆-SSGENLYFQ-GGG-IL15Rα-IL15 (207) and Inclusion Body Isolation

Expression of His₆-SSGENLYFQ-GGG-IL15Rα-IL15 207 starts with the transformation of the plasmid (pET32a-IL15Rα-lL15) into BL21 cells (Novagen). Next step was the inoculation of 500 mL culture (LB medium + ampicillin) with BL21 cells. When OD600 reached 0.7, cultures were induced with 1 mM IPTG (500 µL of 1 M stock solution). After 4 hour induction at 37° C., the culture was pelleted by centrifugation. The cell pellet gained from 500 mL culture was lysed in 25 mL BugBuster™ with 625 units of benzonase and incubated on roller bank for 20 min at room temperature. After lysis the insoluble fraction was separated from the soluble fraction by centrifugation (20 minutes, 12000 × g, 4° C.). The insoluble fraction was dissolved in 25 mL BugBuster™ with lysozyme (final concentration: 200 µg/mL) and incubated on the roller bank for 5 min. Next the solution was diluted with 6 volumes of 1:10 diluted BugBuster™ and centrifuged 15 min, 9000 x g at 4° C. The pellet was resuspended in 250 mL of 1:10 diluted BugBuster™ by using the homogenizer and centrifuged at 15 min, 9000 x g at 4° C. The last step was repeated 3 times.

### Example 142. Refolding of His₆-SSGENLYFQ-GGG-IL15Rα-IL15 207 From Isolated Inclusion Bodies

The purified inclusion bodies containing His₆-SSGENLYFQ-GGG-IL15Rα-IL15 207, were sulfonated o/n at 4° C. in 25 mL denaturing buffer (5 M guanidine, 0.3 M sodium sulphite) and 2.5 mL 50 mM disodium 2-nitro-5-sulfobenzonate. The solution was diluted with 10 volumes of cold Milli-Q and centrifuged (10 min at 8000 x g). The pellet was solved in 125 mL cold Milli-Q using a homogenizer and centrifuged (10 min at 80000 x g). The last step was repeated 3 times. The purified His₆-SSGENLYFQ-GGG-IL15Rα-IL15 207 was denatured in 5 M guanidine and diluted to a concentration of 1 mg/mL of protein. Using a syringe with a diameter of 0.8 mm, the denatured protein was added dropwise to 10 volumes refolding buffer (50 mM Tris, 10.53 mM NaCl, 0.44 mM KCI, 2.2 mM MgCl₂, 2.2 mM CaCl₂, 0.055% PEG-4000, 0.55 M L-arginine, 8 mM cysteamine, 4 mM cystamine, at pH 8.0) on ice and was incubate 48 hours at 4° C. (stirring not required). The refolded His₆-SSGENLYFQ-GGG-IL15Rα-IL15 207 was loaded on a 20 mL HisTrap excel column (GE health care) on an AKTA Purifier-10 (GE Healthcare). The column was first washed with buffer A (5 mM Tris buffer, 20 mM imidazole, 500 mM NaCl, pH 7.5). Retained protein was eluted with buffer B (20 mM Tris buffer, 500 mM imidazole, 500 mM NaCl, pH 7.5) on a gradient of 25 mL from buffer A to buffer B. Fractions were analysed by SDS-PAGE on polyacrylamide gels (16%). The fractions that contained purified target protein were combined and the buffer was exchanged against TBS (20 mM Tris pH 7.5 and 150 mM NaCl₂) by dialysis performed overnight at 4° C. The purified protein was concentrated to at least 2 mg/mL using Amicon Ultra-0.5, MWCO 3 kDa (Merck-Millipore). Mass spectral analysis showed a weight of 25044 Da (expected: 25044 Da). The product was stored at -80° C. prior to further use.

### Example 143. TEV Cleavage of His₆-SSGENLYFQ-GGG-IL15Rα-IL15 207 to Obtain GGG-IL15Rα-IL15 208

To a solution of His₆-SSGENLYFQ-GGG-IL15Rα-IL15 (207, 330 µL, 2.3 mg/mL in TBS pH 7.5), was added TEV protease (50.5 µL, 10 Units/µL in 50 mM Tris-HCl, 250 mM NaCl, 1 mM TCEP, 1 mM EDTA, 50% glycerol, pH 7.5, New England Biolabs). The reaction was incubated for 1 hour at 30° C. After TEV cleavage, the solution was purified using size exclusion chromatography. The reaction mixture was loaded on to a Superdex 75 10/300 GL column (GE Healthcare) on an AKTA Purifier-10 (GE Healthcare) using TBS pH 7.5 as mobile phase and a flow of 0.5 mL/min. GGG-IL15Rα-IL15 208 was eluted at a retention time of 12 mL. The purified protein was concentrated to at least 2 mg/mL using an Amicon Ultra-0.5, MWCO 3 kDa (Merck Millipore). The product was analysed with mass spectrometry (observed mass: 22965 Da, expected mass: 22964 Da), corresponding to GGG-IL15Rα-IL15 208. The product was stored at -80° C. prior to further use.

### Example 144. Incorporation of BCN-PEG₁₂-LPETGG (168) in GGG-IL15Rα-IL15 208 Using Sortase A to Obtain BCN-PEG₁₂-IL15Rα-IL15 (209)

To a solution of GGG-IL15Rα-IL15 (208, 219 µL, 91.4 µM in TBS pH 7.5) was added TBS pH 7.5 (321 µL), CaCl₂ (40.0 µL, 100 mM) and BCN-PEG₁₂-LPETGG (168, 120 µL, 5 mM in DMSO) and incubated 1 hour at 37° C. After incorporation of 168 was complete, sortase A was removed from the solution using the same volume of Ni-NTA beads as reaction volume (800 µL). The solution was incubated for 1 hour in a spinning wheel/or table shaker, afterwards the solution was centrifuged (2 min, 13000 rpm) and the supernatant was discarded. BCN-PEG₁₂-IL15Rα-IL15 (209) was collected from the beads by incubating the beads 5 min with 800 µL washing buffer (40 mM imidazole, 20 mM Tris, 0.5 M NaCl) in a table shaker at 800 rpm. The beads were centrifuged (2 min, 13000 × rpm), the supernatant containing 209 was separated and the buffer was exchanged to TBS by dialysis o/n at 4° C. Finally, the solution was concentrated to 0.5-1 mg/mL using Amicon spin filter 0.5, MWCO 3 kDa (Merck-Millipore). Mass spectrometry analysis showed a weight of 24155 Da (expected mass: 24152) corresponding to BCN-PEG₁₂-IL15Rα-IL15 (209).

### Example 145. Conjugation of BCN-PEG₁₂-IL15Rα-IL15 (209) to Trastuzumab(6-N₃-GaINAc)₂ 205 to Obtain Conjugate 210

A bioconjugate according to the invention was prepared by conjugation of 209 to azide-modified trastuzumab (205, trastuzumab(6-N₃-GaINAc)₂, prepared according to WO2016170186) in a 2:1 molar ratio. Thus, to a solution of BCN-PEG₁₂-IL15Rα-IL15 (209, 20 µL, 20 µM in TBS pH 7.4) was added trastuzumab(6-N₃-GaINAc)₂ (205, 1.2 µL, 82 µM in PBS pH 7.4) and incubated o/n at 37° C. Mass spectral analysis of the IdeS-digested sample showed a mass of 48526 Da (expected mass: 48518 Da) corresponding to the Fc/2-fragment of conjugate 210.

### Example 146. Intramolecular Cross-Linking of Trastuzumab-(azide)₂ With Bivalent Linker 105 to Give 211

To a solution of trastuzumab-(6-azidoGaINAc)₂ (7.5 µL, 150 µg, 17.56 mg/mL in PBS pH 7.4; also referred to as trast-v1a), prepared according to WO2016170186, was added compound 105 (2.5 µL, 0.8 mM solution in DMF, 2 equiv. compared to IgG). The reaction was incubated for 1 day at RT followed by buffer exchange to PBS pH 7.4 using centrifugal filters (Amicon Ultra-0.5 mL MWCO 10 kDa, Merck-Millipore). Mass spectral analysis of the IdeS digested sample showed one major product (calculated mass 49625 Da, observed mass 49626 Da), corresponding to intramolecularly cross-linked trastuzumab derivative 211. HPLC-SEC showed <4% aggregation, hence excluding intermolecular cross-linking.

### Example 147. Intramolecular Cross-Linking of Trastuzumab-(azide)₂ With Bivalent Linker 107 to Give 212

To a solution of trastuzumab-(6-azido-GaINAc)₂ (7.5 µL, 150 µg, 17.56 mg/mL in PBS pH 7.4) was added compound 107 (2.5 µL, 4 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck-Millipore). Mass spectral analysis of the IdeS digested sample showed the product (calculated mass 50153 Da, observed mass 50158 Da), corresponding to intramolecularly cross-linked trastuzumab derivative 212. HPLC-SEC showed <4% aggregation, hence excluding intermolecular cross-linking.

### Example 148. Intramolecular Cross-Linking of Trastuzumab-(azide)₂ With Bivalent Linker 117 to Give 213

To a solution of trastuzumab-(6-azidoGaINAc)₂ (7.5 µL, 150 µg, 17.56 mg/mL in PBS pH 7.4) was added compound 117 (2.5 µL, 0.8 mM solution in DMF, 2 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49580 Da, observed mass 49626 Da), corresponding to intramolecularly cross-linked trastuzumab derivative 213. HPLC-SEC showed <4% aggregation, hence excluding intermolecular cross-linking.

### Example 149. Intramolecular Cross-Linking of Trastuzumab-(Azide)₂ With Bivalent Linker 118 to Give 214

To a solution of trastuzumab-(6-azidoGaINAc)₂ (7.5 µL, 150 µg, 17.56 mg/mL in PBS pH 7.4) was added compound 118 (2.5 µL, 4 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed the product (calculated mass 49358 Da, observed mass 49361 Da), corresponding to intramolecularly cross-linked trastuzumab derivative 214. HPLC-SEC showed <4% aggregation, hence excluding intermolecular cross-linking.

### Example 150. Intramolecular Cross-Linking of Trastuzumab-(azide)₂ With bivalent Linker 124 to Give 215

To a solution of trastuzumab-(6-azidoGaINAc)₂ (7.5 µL, 150 µg, 17.56 mg/mL in PBS pH 7.4) was added compound 124 (2.5 µL, 4 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed the product (calculated mass 49406 Da, observed mass 49409 Da), corresponding to intramolecularly cross-linked trastuzumab derivative 215. HPLC-SEC showed <4% aggregation, hence excluding intermolecular cross-linking.

### Example 151. Intramolecular Cross-Linking of Trastuzumab-(azide)₂ With Bivalent Linker 125 to Give 216

To a solution of trastuzumab-(6-azidoGaINAc)₂ (7.5 µL, 150 µg, 17.56 mg/mL in PBS pH 7.4) was added compound 125 (2.5 µL, 0.8 mM solution in DMF, 2 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49184 Da, observed mass 49184 Da), corresponding to intramolecularly cross-linked trastuzumab derivative 216. HPLC-SEC showed <4% aggregation, hence excluding intermolecular cross-linking.

### Example 152. Intramolecular Cross-Linking of Trastuzumab-(Azide)₂ With Bivalent Linker 145 to Give 217

To a solution of trastuzumab-(6-azidoGaINAc)₂ (320 µL, 2 mg, 5.56 mg/mL in PBS pH 7.4) was added compound 145 (80 µL, 1.66 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49796 Da, observed mass 49807 Da), corresponding to intramolecularly cross-linked trastuzumab derivative 217. HPLC-SEC showed <4% aggregation, hence excluding intermolecular cross-linking.

### Example 153. Intramolecular Cross-Linking of Trastuzumab-(Azide)₂ With Bivalent Linker-Payload Construct 137 to Give DAR1 ADC 218

To a solution of trastuzumab-(6-azidoGaINAc)₂ (37.5 µL, 250 µg, 6.67 mg/mL in PBS pH 7.4) was added compound 137 (12.5 µL, 0.67 mM solution in DMF, 5 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 50464 Da, observed mass 50474 Da), corresponding to the conjugated ADC 218 obtained via intramolecular cross-linking. HPLC-SEC showed <4% aggregation, hence excluding intermolecular cross-linking. RP-HPLC showed the Fc/2 (tᵣ 6.099), Fc-toxin (tᵣ 8.275, corresponding to 82.4% of total Fc/2 fragments) and Fab (tᵣ 9.320) fragments.

### Example 154. Intramolecular Cross-Linking of Trastuzumab-(Azide)₂ With Bivalent Linker-Payload Construct 131 to Give DAR1 ADC 219

To a solution of trastuzumab-(6-azidoGaINAc)₂ (37.5 µL, 250 µg, 6.67 mg/mL in PBS pH 7.4) was added compound 131 (12.5 µL, 0.67 mM solution in DMF, 5 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 50638 Da, observed mass 50649 Da), corresponding to the ADC 219 obtained via intramolecular cross-linking. HPLC-SEC showed <4% aggregation, hence excluding intermolecular cross-linking. RP-HPLC showed the Fc/2 (tᵣ 6.082), Fc-toxin (tᵣ 9.327, corresponding to 76.7% of total Fc/2 fragments) and Fab (tᵣ 9.347) fragments.

### Example 155. Intramolecular Cross-Linking of Trastuzumab-(azide)₂ With Bivalent Linker-Payload Construct 139 to Give DAR1 ADC 220

To a solution of trastuzumab-(6-azidoGaINAc)₂ (37.5 µL, 250 µg, 6.67 mg/mL in PBS pH 7.4) was added compound 139 (12.5 µL, 0.67 mM solution in DMF, 5 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 50392 Da, observed mass 50402 Da), corresponding to the ADC 220 obtained via intramolecular cross-linking. HPLC-SEC showed <4% aggregation, hence excluding intermolecular cross-linking. RP-HPLC showed the Fc/2 (tᵣ 6.062), Fc-toxin (tᵣ 8.548, corresponding to 89.5% of total Fc/2 fragments) and Fab (tᵣ 9.295) fragments.

### Example 156. Intramolecular Cross-Linking of Trastuzumab Derivative 217 (containing Single BCN) With Tetrazine-Modified Anti-CD3 Immune Cell Engager 204 to Give T Cell Engager 221 With 2:1 Molecular Format

To a solution of 217 (8 µL, 141 µg, 17.7 mg/mL in PBS pH 7.4) was added hOKT3-PEG₄-tetrazine (204, 13.15 µL, 280 µg, 21.45 mg/mL in PBS pH 7.4, 2 equiv. compared to IgG). Mass spectral analysis of the IdeS-digested sample showed one major product (calculated mass 77664 Da, observed mass 77647 Da), corresponding to the conjugated Fc-PEG₄-hOKT3 (221).

### Example 157. Intramolecular Cross-Linking of Bis-Azido-Rituximab Rit-v1a With Trivalent Linker 145 To Give BCN-Rituximab Rit-v1a-145

To a solution of bis-azido-rituximab rit-v1a (494 µL, 30 mg, 60.7 mg/mL in PBS pH 7.4), prepared according to WO2016170186, was added PBS pH 7.4 (2506 µL), propylene glycol (2980 µL) and trivalent linker 145 (20 µL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Reducing SDS-PAGE showed one major HC product, corresponding to the crosslinked heavy chain (See FIG. 16, right panel, lane 3), indicating formation of rit-v1a-145. Furthermore, non-reducing SDS-PAGE showed one major band around the same height as rit-v1a (See FIG. 16, left panel, lane 3), demonstrating that only intramolecular cross-linking occurred.

### Example 158. Intramolecular Cross-Linking of bis-azido-B12 B12-v1a With Trivalent Linker 145 to Give BCN-B12 B12-v1a-145

To a solution of bis-azido-B12 B12-v1a (415 µL, 4 mg, 9.6 mg/mL in PBS pH 7.4), prepared according to WO2016170186, was added propylene glycol (412 µL) and trivalent linker 145 (2.7 µL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. RP-HPLC analysis of an IdeS-digested sample shows formation of B12-v1a-145. (See FIG. 17).

### Example 159. Intramolecular Cross-Linking of Bis-Azido-Trastuzumab Trast-v1a With bis-BCN-TCO 5 XL11 to give TCO-Trastuzumab Trast-v1a-XL11

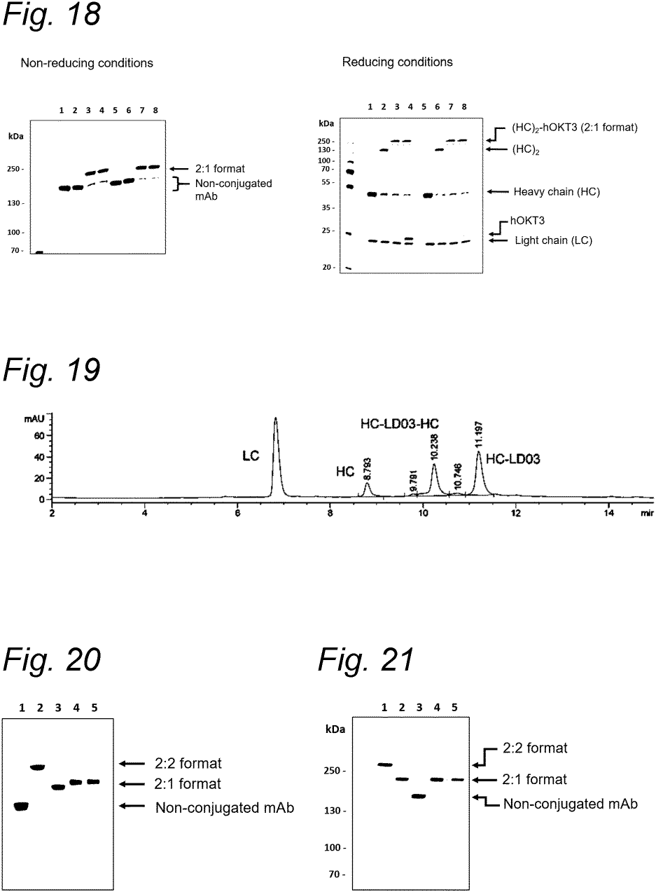

To a solution of bis-azido-trastuzumab trast-v1a (36 µL, 2 mg, 56.1 mg/mL in PBS pH 7.4), prepared according to WO2016170186, was added PBS pH 7.4 (164 µL), propylene glycol (195 µL) and bis-BCN-TCO XL11 (5.3 µL, 10 mM solution in DMF, 4.0 equiv. compared to IgG). The reaction was incubated overnight at rt followed by buffer exchange to PBS pH 7.4 using centrifugal filters (Amicon Ultra-0.5 mL MWCO 10 kDa, Merck Millipore). Reducing SDS-PAGE showed two major HC products, corresponding to the nonconjugated heavy chain and the crosslinked heavy chain (See FIG. 18, right panel, lane 2), indicating partial conversion into trast-v1a-XL11. Furthermore, non-reducing SDS-PAGE showed one major band at the height of trast-v1a (See FIG. 18, left panel, lane 2), indicating that only intramolecular crosslinking occurred.

### Example 160. Intramolecular Cross-Linking of Bis-Azido-Rituximab Rit-v1a With Bis-BCN-TCO XL11 to Give TCO-Rituximab rit-v1a-XL11

To a solution of bis-azido-rituximab rit-v1a (37 µL, 2 mg, 54.5 mg/mL in PBS pH 7.4) was added PBS pH 7.4 (163 µL), propylene glycol (195 µL) and bis-BCN-TCO XL11 (5.3 µL, 10 mM solution in DMF, 4.0 equiv. compared to IgG). The reaction was incubated overnight at rt followed by buffer exchange to PBS pH 7.4 using centrifugal filters (Amicon Ultra-0.5 mL MWCO 10 kDa, Merck Millipore). Reducing SDS-PAGE showed two major HC products, corresponding to the nonconjugated heavy chain and the crosslinked heavy chain (See FIG. 18, right panel, lane 6), indicating partial conversion into rit-v1a-XL11. Furthermore, non-reducing SDS-PAGE showed one major band at the height of rit-v1a (See FIG. 18, left panel, lane 2), indicating that only intramolecular crosslinking occurred.

### Example 161. Intramolecular Cross-Linking of Bis-Azido-Trastuzumab Trast-v3 With Bis-BCN-MMAE 137 to Give DAR1 ADC Trast-v3-137

To a solution of trast-v3 (15 µL, 150 µg, 10 mg/mL in PBS pH 7.4) was added bis-BCN-MMAE (137, 15 µL, 0.27 mM solution in PG, 4 eq compared to IgG). The reaction was incubated for 16 hours at room temperature followed by buffer exchange to PBS pH 7.4 using centrifugal filters (Amicon Ultra-0.5 mL MWCO 10 kDa, Merck Millipore). Mass spectral analysis of the IdeS digested sample showed one major product (observed mass 49719 Da), corresponding to trast-v3-137 obtained via intramolecular cross-linking.

### Example 162. Intramolecular Cross-Linking of Deglycosylated Trastuzumab With Bis-BCN-MMAE LD03

Deglycosylated trastuzumab (8.3 µL, 0.15 mg, 18.1 mg/mL in PBS 5.5) was incubated with bis-BCN-MMAE (LD03, 8.3 µL, 1.2 mM in PG) and mushroom tyrosinase (3 µL, 10 mg/mL in phosphate buffer pH 6.0, Sigma Aldrich T3824) for 16 hours at room temperature. See also Dutch patent application no. 2026947, incorporated by reference herein. RP-HPLC analysis of DTT treated ADC showed 35% conversion into trast-v4-LD03 (see FIG. 19).

### Example 163. Intramolecular Cross-Linking of Bis-Azido-Trastuzumab Trast-v3 With Bis-BCN-MMAE LD03 to Give DAR1 ADC Trast-v3-LD03

To a solution of trast-v3 (22.5 µL, 5 mg, 6.7 mg/mL in PBS pH 7.4) was added bis-BCN-MMAE (LD03, 7.5 µL, 0.53 mM solution in DMF, 4 eq compared to IgG). The reaction was incubated for 16 hours at room temperature followed by buffer exchange to PBS pH 7.4 using centrifugal filters (Amicon Ultra-0.5 mL MWCO 10 kDa, Merck Millipore). Mass spectral analysis of the IdeS digested sample showed one major product (observed mass 50052 Da), corresponding to trast-v3-LD03 obtained via intramolecular cross-linking.

### Example 164. Intramolecular Cross-Linking of Bis-Azido-Rituximab Rit-v3 With Bis-BCN-MMAE LD03 to Give DAR1 ADC rit-v3-LD03

To a solution of rit-v3 (22.5 µL, 5 mg, 6.7 mg/mL in PBS pH 7.4) was added bis-BCN-MMAE (LD03, 7.5 µL, 0.53 mM solution in DMF, 4 eq compared to IgG). The reaction was incubated for 16 hours at room temperature followed by buffer exchange to PBS pH 7.4 using centrifugal filters (Amicon Ultra-0.5 mL MWCO 10 kDa, Merck Millipore). Mass spectral analysis of the IdeS digested sample showed one major product (mass 49989 Da), corresponding to rit-v3-LD03 obtained via intramolecular cross-linking.

### Example 165. Intramolecular Cross-Linking of Bis-BCN-IL15Rα-IL15 PF27 to Trast-v3 via Strain-Promoted Alkyne-Azide Cycloaddition (SPAAC) (P:A ratio 1:1)

Trast-v3 (2.57 µL, 0.05 mg, 19.5 mg/mL in PBS) was incubated with bis-BCN-IL15Rα-IL15 (PF27, 5.6 µL, 3 eq. bis-BCN labelled IL15Rα-IL15, 7.6 mg/mL in PBS) for 16 hours at room temperature. Mass spectral analysis of a sample after IdeS treatment showed one major Fc/2 product (observed mass 73432 Da) corresponding to the expected product trast-v3-PF27.

### Example 166. Intramolecular Cross-Linking of hOKT3-Bis-BCN PF22 to Trast-v3 via SPAAC (P:A Ratio 1:1)

Trast-v3 (2.57 µL, 0.05 mg, 19.5 mg/mL in PBS) was incubated with hOKT3-bis-BCN PF22 (5.15 µL, 3 eq., 5.7 mg/mL in PBS) for 16 hours at room temperature. Mass spectral analysis of a sample after IdeS treatment showed one major Fc/2 product (observed mass 77150 Da) corresponding to the expected product trast-v3-PF22.

### Example 167. Conjugation of hOKT3-PEG₄-tetrazine 204 to BCN-Rituximab Rit-v1a-145 to Give T Cell Engager rit-v1a-145-204 with 2:1 Molecular Format

To a solution of rit-v1a-145 (287 µL, 6.6 mg, 154 µM in PBS pH 7.4) was added hOKT3-PEG₄-tetrazine 204 (247 µL, 1.9 mg, 269 µM in PBS pH 6.5, 1.5 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (See FIG. 16, left panel, lane 5), thereby confirming formation of rit-v1a-145-204. Furthermore, reducing SDS-PAGE confirms one major HC product, corresponding to two heavy chains conjugated to a single hOKT3 (See FIG. 16, right panel, lane 5).

### Example 168. Conjugation of hOKT3-PEG₁₁-tetrazine PF01 to BCN-Rituximab rit-v1a-145 to Give T Cell Engager rit-v1a-145-PF01 With 2:1 Molecular Format

To a solution of rit-v1a-145 (247 µL, 6.3 mg, 171 µM in PBS pH 7.4) was added hOKT3-PEG₁₁-tetrazine PF01 (304 µL, 2.0 mg, 230 µM in PBS pH 6.5, 1.7 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (See FIG. 16, left panel, lane 6), thereby confirming formation of rit-v1a-145-PF01. Furthermore, reducing SDS-PAGE confirms one major HC product, corresponding to two heavy chains conjugated to a single hOKT3 (See FIG. 16, right panel, lane 6).

### Example 169. Conjugation of hOKT3-PEG₁₁-tetrazine PF01 to BCN-B12 B12-v1a-145 to Give T cell Engager B12-v1a-145-PF01 With 2:1 Molecular Format

To a solution of B12-v1a-145 (38 µL, 1.0 mg, 178 µM in PBS pH 7.4) was added hOKT3-PEG₁₁-tetrazine PF01 (44 µL, 0.3 mg, 230 µM in PBS pH 6.5, 1.5 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (see FIG. 20, lane 4), thereby confirming formation of B12-v1a-145-PF01.

### Example 170. Conjugation of hOKT3-PEG₄-tetrazine 204 to TCO-Trastuzumab Trast-v1a-XL11 to Give T Cell Engager Trast-v1a-XL11-204 With 2:1 Molecular Format

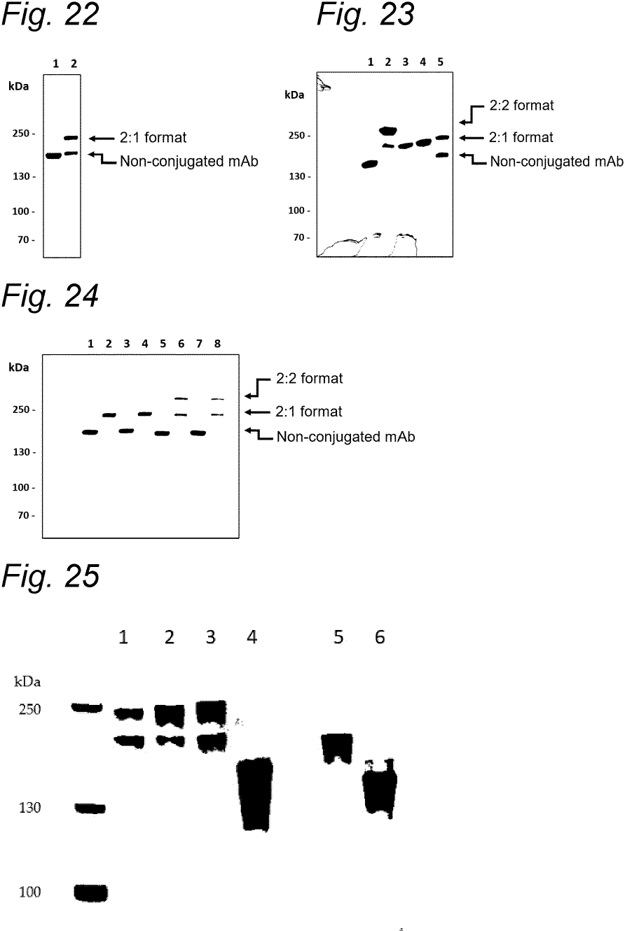

To a solution of TCO-trastuzumab trast-v1a-XL11 (5.7 µL, 100 µg, 117 µM in PBS pH 7.4) was added hOKT3-PEG₄-tetrazine 204 (5 µL, 38 µg, 269 µM in PBS pH 6.5, 2.0 equiv. compared to IgG). The reaction was incubated overnight at rt. Non-reducing SDS-PAGE analysis showed two major products corresponding to the non-conjugated antibody and the antibody conjugated to a single hOKT3 (See FIG. 22, left panel, lane 3), thereby confirming formation of trast-v1a-XL11-204. Furthermore, reducing SDS-PAGE confirms that OKT3 is conjugated to the crosslinked heavy chains containing the TCO reactive handle (See FIG. 22, right panel, lane 3).

### Example 171. Conjugation of hOKT3-PEG₄-tetrazine 204 to TCO-rituximab rit-v1a-XL11 to Give T Cell Engager rit-v1a-XL11-204 With 2:1 Molecular Format

To a solution of TCO-rituximab rit-v1a-XL11 (56.3 µL, 100 µg, 106 µM in PBS pH 7.4) was added hOKT3-PEG₄-tetrazine 204 (5 µL, 38 µg, 269 µM in PBS pH 6.5, 2.0 equiv. compared to IgG). The reaction was incubated overnight at rt. Non-reducing SDS-PAGE analysis showed two major products corresponding to the non-conjugated antibody and the antibody conjugated to a single hOKT3 (See FIG. 22, left panel, lane 7), thereby confirming formation of rit-v1a-XL11-204. Furthermore, reducing SDS-PAGE confirms that OKT3 is conjugated to the crosslinked heavy chains containing the TCO reactive handle (See FIG. 22, right panel, lane 7).

### Example 172. Conjugation of hOKT3-PEG₂₃-Tetrazine PF02 to BCN-Rituximab rit-v1a-145 to Give T Cell Engager rit-v1a-145-PF02 With 2:1 Molecular Format

To a solution of rit-v1a-145 (247 µL, 6.3 mg, 171 µM in PBS pH 7.4) was added hOKT3-PEG₂₃-tetrazine PF02 (262 µL, 2.0 mg, 267 µM in PBS pH 6.5, 1.7 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (See FIG. 18, left panel, lane 7), thereby confirming formation of rit-v1a-145-PF02. Furthermore, reducing SDS-PAGE confirms one major HC product, corresponding to two heavy chains conjugated to a single hOKT3 (See FIG. 18, right panel, lane 7).

### Example 173. Conjugation of hOKT3-PEG₂-arylazide PF03 to BCN-Trastuzumab Trast-v1a-145 to Give T Cell Engager Trast-v1a-145-PF03 With 2:1 Molecular Format

To a solution of trast-v1a-145 (2.9 µL, 150 µg, 347 µM in PBS pH 7.4) was added hOKT3-PEG₂-arylazide PF03 (4.9 µL, 56 µg, 411 µM in PBS pH 7.4, 2.0 equiv. compared to IgG). The reaction was incubated overnight at rt. Mass spectral analysis of the reduced sample showed one major heavy chain product (observed mass 128388 Da), corresponding to trast-v1a-145-PF03.

### Example 174. Conjugation of hOKT3-PEG₂-arylazide PF03 to BCN-Rituximab Rit-v1a-145 to Give T Cell Engager Rit-v1a-145-PF03 With 2:1 Molecular Format

To a solution of rit-v1a-145 (30 µL, 1.5 mg, 337 µM in PBS pH 7.4) was added hOKT3-PEG₂-arylazide PF03 (49 µL, 0.6 mg, 411 µM in PBS pH 7.4, 2.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Mass spectral analysis of the reduced sample showed one major heavy chain product (observed mass 128211 Da), corresponding to rit-v1a-145-PF03.

### Example 175. Conjugation bis-BCN-hOKT3 PF22 to Bis-Azido-Trastuzumab Trast-v1a to Give T Cell Engager Trast-v1a-PF22 With 2:1 Molecular Format

To a solution of trast-v1a (1.8 µL, 100 µg, 374 µM in PBS pH 7.4) was added PBS pH 7.4 (4.5 µL) and bis-BCN-hOKT3 PF22 (13.7 µL, 78 µg, 194 µM in PBS pH 7.4, 4.0 equiv. compared to IgG). The reaction was incubated overnight at rt. Non-reducing SDS-PAGE analysis showed one major product consisting of an antibody conjugated to a single hOKT3 (See FIG. 21, lane 5), thereby confirming formation of trast-v1a-PF22.

### Example 176. Conjugation of bis-BCN-hOKT3 PF22 to Bis-azido-Rituximab Rit-v1a to Give T Cell Engager Rit-v1a-145-PF22 With 2:1 Molecular Format

To a solution of rit-v1a (1.8 µL, 100 µg, 363 µM in PBS pH 7.4) was added PBS pH 7.4 (7.9 µL) and bis-BCN-hOKT3 PF22 (10.3 µL, 58 µg, 194 µM in PBS pH 7.4, 3.0 equiv. compared to IgG). The reaction was incubated overnight at rt. Non-reducing SDS-PAGE analysis showed one major product consisting of an antibody conjugated to a single hOKT3 (See FIG. 21, lane 4), thereby confirming formation of rit-v1a-PF22.

### Example 177. Conjugation of bis-BCN-hOKT3 PF23 to Bis-Azido-Trastuzumab Trast-v1a to Give T Cell Engager Trast-v1a-Pf23 With 2:1 Molecular Format

To a solution of trast-v1a (1.8 µL, 100 µg, 374 µM in PBS pH 7.4) was added PBS pH 7.4 (9.9 µL) and bis-BCN-hOKT3 PF23 (8.4 µL, 58 µg, 239 µM in PBS pH 7.4, 3.0 equiv. compared to IgG). The reaction was incubated overnight at 37° C. Non-reducing SDS-PAGE analysis showed two major products consisting of non-conjugated trastuzumab and trastuzumab conjugated to bis-BCN-hOKT3 PF23 (See FIG. 22, lane 2), thereby confirming partial formation of trast-v1a-PF23.

### Example 178. Conjugation of bis-BCN-hOKT3 PF23 to Bis-Azido-Rituximab Rit-v1a to Give T Cell Engager rit-v1a-PF23 With 2:1 Molecular Format

To a solution of rit-v1a (1.8 µL, 100 µg, 363 µM in PBS pH 7.4) was added PBS pH 7.4 (13.6 µL) and bis-BCN-hOKT3 PF23 (4.3 µL, 30 µg, 239 µM in PBS pH 7.4, 1.5 equiv. compared to IgG). The reaction was incubated overnight at 37° C. Non-reducing SDS-PAGE analysis showed two major products consisting of non-conjugated rituximab and rituximab conjugated once to bis-BCN-hOKT3 PF23 (See FIG. 23, lane 5), thereby confirming partial formation of rit-v1a-PF23.

### Example 179. Conjugation of 4-1BB-PEG₁₁-tetrazine PF08 to BCN-rituximab rit-v1a-145 to Give T Cell Engager rit-v1a-145-PF08 With 2:1 Molecular Format

To a solution of rit-v1a-145 (35 µL, 0.9 mg, 170 µM in PBS pH 7.4) was added 4-1BB-PEG₁₁-tetrazine PF08 (40 µL, 248 µg, 222 µM in PBS pH 7.4, 1.5 equiv. compared to IgG). The reaction was incubated overnight at rt. Non-reducing SDS-PAGE analysis showed one major product consisting of rituximab conjugated to 4-1BB-PEG₂₃-BCN PF08 (See FIG. 20, lane 3), thereby confirming partial formation of rit-v1a-145-PF08.

### Example 180. Conjugation of 4-1BB-PEG₁₁-Tetrazine PF08 to BCN-B12 B12-v1a-145 to Give T Cell Engager B12-v1a-145-PF08 With 2:1 Molecular Format

To a solution of B12-v1a-145 (34 µL, 0.9 mg, 178 µM in PBS pH 7.4) was added 4-1BB-PEG₁₁-tetrazine PF08 (40 µL, 248 µg, 222 µM in PBS pH 7.4, 1.5 equiv. compared to IgG). The reaction was incubated overnight at rt. Non-reducing SDS-PAGE analysis showed one major product consisting of B12 conjugated to 4-1BB-PEG₂₃-BCN PF08 (See FIG. 20, lane 5), thereby confirming partial formation of B12-v1a-145-PF08.

### Example 181. Conjugation of 4-1BB-PEG₂-arylazide PF09 to BCN-Trastuzumab trast-v1a-145 to Give T Cell Engager trast-v1a-145-PF09 With 2:1 Molecular Format

To a solution of trast-v1a-145 (1.9 µL, 100 µg, 347 µM in PBS pH 7.4) was added 4-1BB-PEG₂-arylazide PF09 (5.9 µL, 37 µg, 225 µM in PBS pH 7.4, 2.0 equiv. compared to IgG). The reaction was incubated overnight at rt. Non-reducing SDS-PAGE analysis showed one major product consisting of trastuzumab conjugated to a single 4-1BB-PEG₂-arylazide PF09 (See FIG. 24, lane 4), thereby confirming formation of trast-v1a-145-PF09.

### Example 182. Conjugation of 4-1BB-PEG₂-arylazide PF09 to BCN-Rituximab Rit-v1a-145 to Give T Cell Engager rit-v1a-145-PF09 with 2:1 Molecular Format

To a solution of rit-v1a-145 (2.0 µL, 100 µg, 337 µM in PBS pH 7.4) was added 4-1BB-PEG₂-arylazide PF09 (5.9 µL, 37 µg, 225 µM in PBS pH 7.4, 2.0 equiv. compared to IgG). The reaction was incubated overnight at rt. Non-reducing SDS-PAGE analysis showed one major product consisting of rituximab conjugated to a single 4-1BB-PEG₂-arylazide PF09 (See FIG. 24, lane 2), thereby confirming formation of rit-v1a-145-PF09.

### Example 183. Conjugation of Tetrazine-PEG₃-GGG-IL15Rα-IL15 (PF12) to BCN-Trastuzumab Trast-v1a-145 to Give T Cell Engager trast-v1a-145-PF12 With 2:1 Molecular Format

Trast-v1a-145 (75 µL, 1.575 mg, 21 mg/mL in PBS) was incubated with PF12 (80 µL, 2 eq., 6.5 mg/mL in PBS) for 16 h at 37° C. Analysis on non-reducing SDS-PAGE confirmed the formation of Trast-v1a-145-PF12 (see FIG. 25, lane 5).

### Example 184. Conjugation of Arylazide-PEG11-GGG-IL15Ra-IL15 (PF13) to BCN-Trastuzumab Trast-v1a-145 to Give T Cell Engager Trast-v1a-145-PF13 With 2:1 Molecular Format

Trast-v1a-145 (280 µL, 5.2 mg, 18.6 mg/mL in PBS) was incubated with PF13 (477 µL, 1.5 eq., 2.6 mg/mL in PBS) for 16 h at 37° C. Mass spectral analysis of a sample after IdeS treatment showed one major product of 73991 Da, corresponding to the crosslinked Fc-fragment conjugated to PF13 (expected mass: 73989 Da), thereby confirming formation of trast-v1a-145-PF13.

### Example 185. Conjugation of Arylazide-PEG11-GGG-IL15Rα-IL15 (PF13) to BCN-Rituximab Rit-v1a-145 to Give T Cell Engager Rit-v1a-145-PF13 With 2:1 Molecular Format

Rit-v1a-145 (0.5 µL, 0.025 mg, 50.6 mg/mL in PBS) was incubated with PF13 (6.6 µL, 4 eq., 2.6 mg/mL in PBS) for 16 h at RT. Mass spectral analysis of a sample after IdeS treatment showed one major product of 73927 Da, corresponding to the crosslinked Fc-fragment conjugated to PF13 (expected mass: 73925 Da), thereby confirming formation of rit-v1a-145-PF13.

### Example 186. Conjugation of bis-BCN-SYR-(G₄S)₃-IL15Rα-IL15 (PF27) to Bis-Azido-Trastuzumab Trast-v1a to Give T Cell Engager Trast-v1a-145-PF27 With 2:1 Molecular Format

Trast-v1a (1.78 µL, 0.099 mg, 56.1 mg/mL in PBS) was incubated with PF27 (18.4 µL, 4 eq., 7.62 mg/mL in PBS) and with 2.87 µL PBS for 16 h at 37° C. Mass spectral analysis of a sample after IdeS treatment showed one major product of 74193 Da, corresponding to the crosslinked Fc- fragment conjugated to PF27 (expected mass: 74178 Da), thereby confirming formation of trast- v1a-145-PF27.

### Example 187. Conjugation of bis-BCN-SYR-(G₄S)₃-IL15Rα-IL15 (PF27) to Bis-Azido-Rituximab Rit-v1a to Give T Cell Engager Rit-v1a-145-PF27 With 2:1 Molecular Format

Rit-v1a (1 µL, 0.055 mg, 54.6 mg/mL in PBS) was incubated with PF27 (8.9 µL, 4 eq.,6.2 mg/mL in PBS) and with 1.6 µL PBS for 16 h at 37° C. Mass spectral analysis of a sample after IdeS treatment showed one major product of 74118 Da, corresponding to the crosslinked Fc-fragment conjugated to PF27 (Expected mass: 74114 Da), thereby confirming formation of rit-v1a-145-PF27.

### Example 188. Conjugation of Azido-IL15Rα-IL15 PF17 to BCN-Trastuzumab Trast-v1a-145 to Give T Cell Engager Trast-v1a-145-PF17 With 2:1 Molecular Format

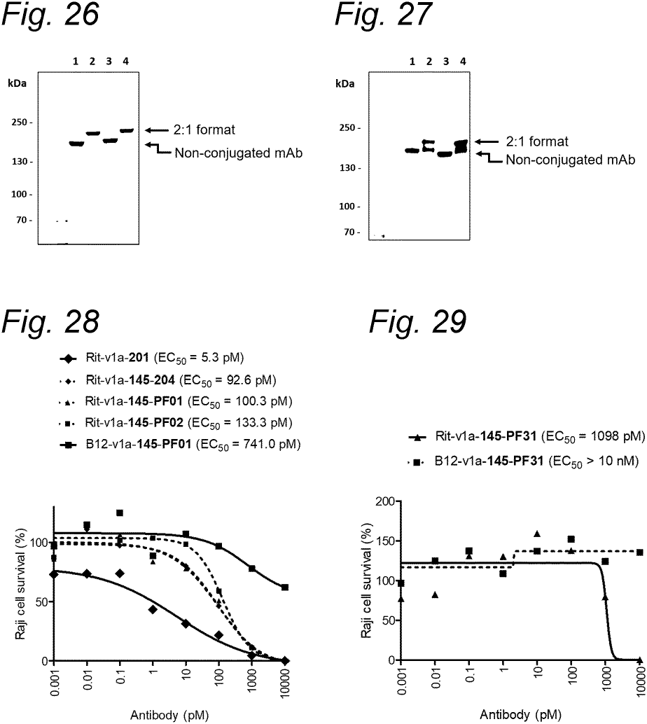

To a solution of trast-v1a-145 (29 µL, 1.5 mg, 347 µM in PBS pH 7.4) was added azido-IL15Rα-IL15 PF17 (97 µL, 1.1 mg, 411 µM in PBS pH 7.4, 4.0 equiv. compared to IgG). The reaction was incubated overnight at 37° C. Non-reducing SDS-PAGE analysis showed one major product consisting of trastuzumab conjugated to a single azido-IL15Rα-IL15 PF17 (See FIG. 26, lane 4), thereby confirming formation of trast-v1a-145-PF17.

### Example 189. Conjugation of Azido-IL15Rα-IL15 PF17 to BCN-Rituximab Rit-v1a-145 to Give T Cell Engager Rit-v1a-145-PF17 With 2:1 Molecular Format

To a solution of rit-v1a-145 (3 µL, 150 µg, 337 µM in PBS pH 7.4) was added azido-IL15Rα-IL15 PF17 (9.7 µL, 111 µg, 411 µM in PBS pH 7.4, 4.0 equiv. compared to IgG). The reaction was incubated overnight at 37° C. Non-reducing SDS-PAGE analysis showed one major product consisting of rituximab conjugated to a single azido-IL15Rα-IL15 PF17 (See FIG. 26, lane 2), thereby confirming formation of rit-v1a-145-PF17.

### Example 190. Conjugation of Azido-IL15 PF19 to BCN-Trastuzumab Tras-v1a-145 to Give T Cell Engager Tras-v1a-145-PF19 With 2:1 Molecular Format

Trast-v1a-145 (4.0 µL, 0.075 mg, 18.6 mg/mL in PBS) was incubated with PF19 (4.6 µL, 5 eq., 7.7 mg/mL in PBS) for 16 h at RT. Mass spectral analysis of a sample after IdeS treatment showed one major product of 63941 Da, corresponding to the crosslinked Fc-fragment conjugated to PF19 (Expected mass: 63936 Da), thereby confirming formation of trast-v1a-145-PF19.

### Example 191. Conjugation of azido-IL15 PF19 to BCN-Rituximab Rit-v1a-145 to Give T Cell Engager Rit-v1a-145-PF19 With 2:1 Molecular Format

Rit-v1a-145 (2.0 µL, 0.112 mg, 50.6 mg/mL in PBS) was incubated with PF19 (5.1 µL, 4 eq., 7.7 mg/mL in PBS) for 16 h at RT. Mass spectral analysis of a sample after IdeS treatment showed one major product of 63882 Da, corresponding to the crosslinked Fc-fragment conjugated to PF19 (Expected mass: 63879 Da), thereby confirming formation of rit-v1a-145-PF19.

### Example 192. Conjugation of bis-BCN-SYR-(G₄S)₃-IL15 (PF29) to Bis-Azido-Trastuzumab Tras-v1a to Give T Cell Engager Tras-v1a-PF29 With 2:1 Molecular Format

Trast-v1a (1 µL, 0.056 mg, 56.1 mg/mL in PBS) was incubated with PF29 (11 µL, 4 eq., 3.6 mg/mL in PBS) for 16 h at 37° C. Non-reducing SDS-PAGE analysis showed two major products corresponding to non-conjugated trastuzumab and trastuzumab conjugated to a single bis-BCN-SYR-(G₄S)₃-IL15 PF29 (See FIG. 27, lane 2), thereby confirming partial conversion into Tras-v1a-PF29.

### Example 193. Conjugation of bis-BCN-SYR-(G₄S)₃-IL15 (PF29) to Bis-Azido-Rituximab Rit-v1a to Give T Cell Engager Rit-v1a-PF29 With 2:1 Molecular Format

Rit-v1a (1 µL, 0.055 mg, 54.6 mg/mL in PBS) was incubated with PF29 (11 µL, 4 eq.,3.6 mg/mL in PBS) for 16 h at 37° C. Non-reducing SDS-PAGE analysis showed two major products corresponding to non-conjugated rituximab and rituximab conjugated to a single bis-BCN-SYR-(G₄S)₃-IL15 PF29 (See FIG. 27, lane 4), thereby confirming partial conversion into rit-v1a-PF29.

### Example 194. Conjugation of Tetrazine-PEG₁₂-SYR-(G₄S)₃-IL15 (PF21) to BCN-Trastuzumab Trast-v1a-145 to Give T Cell Engager Trast-v1a-145-PF21 With 2:1 Molecular Format

Trast-v1a (2 µL, 0.042 mg, 21 mg/mL in PBS) was incubated with PF21 (10 µL, 6.7 eq., 2.9 mg/mL in PBS) for 16 h at 37° C. Mass spectral analysis of a sample after IdeS treatment showed one major product of 64865 Da, corresponding to the crosslinked Fc-fragment conjugated to PF21 (Expected mass: 64863 Da), thereby confirming formation of trast-v1a-145-PF21.

### Example 195. CD3 Binding Analysis

Specific binding to CD3 was assessed using Jurkat E6.1 cells, which express CD3 on the cell surface, and MOLT-4 cells, which do not express CD3 on the cell surface. Both cell lines were cultured in RPMI 1640 supplemented with 1% pen/strep and 10% fetal bovine serum at a concentration of 2 × 10⁵ to 1 × 10⁶ cells/ml. Cells were washed in fresh medium before the experiment and 100,000 cells per well were seeded in a 96-wells plate (duplicate wells). The dilution series of 6 antibodies were made in phosphate-buffered saline (PBS). The antibodies were diluted 10 times in the cell suspension and incubated at 4° C. in the dark for 30 minutes. After incubation, the cells were washed twice in cold PBS / 0.5% BSA, and incubated with anti-HIS-PE (only for 200) or anti-lgG1-PE (for all other compounds) at 4° C., in the dark for 30 minutes. After the second incubation step, the cells were washed twice. 7AAD was added as a live-dead staining. Detection of the fluorescence in the Yellow-B channel (anti-IgG1-PE and anti-HIS-PE) and the Red-B channel (7AAD) was done with the Guava 5HT flow cytometer. Median fluorescence intensity in the Yellow-B channel (anti-IgG1-PE and anti-HIS-PE) in life cells was determined with Kaluza software. All bispecifics, but not the negative control rituximab, show concentration-dependent binding to the CD3 positive Jurkat E6.1 cell line (Table 1). In contrast, no binding was observed to the CD3 negative MOLT-4 cell line (Table 2).

TABLE 1

Analysis of antibody binding to CD3-positive cells (Jurkat E6.1) by FACS. The median fluorescence intensity of a duplicate is shown for each concentration tested.

| | | | | | | |
|---|---|---|---|---|---|---|
| Concentration (nM) | Rit-v1a-201 | Rit-v1a-145-204 | Rit-v1a-145-PF01 | Rit-v1a-145-PF02 | Rituximab | 200 |
| 0.32 | 104.79 | 77.80 | 80.30 | 58.79 | 58.28 | 63.63 |
| 1 | 90.00 | 82.15 | 88.23 | 71.52 | 56.55 | 66.22 |
| 3.16 | 159.28 | 112.67 | 116.72 | 114.55 | 55.37 | 83.74 |
| 10 | 160.91 | 113.22 | 142.62 | 168.91 | 60.83 | 109.70 |
| 31.6 | 202.99 | 165.47 | 221.00 | 229.84 | 58.71 | 154.42 |
| 100 | 248.66 | 200.74 | 252.20 | 278.91 | 55.49 | 177.16 |
| 316 | 294.49 | 263.83 | 256.79 | 291.09 | 54.99 | 223.52 |
| 1000 | 420.46 | 315.13 | 366.89 | 355.26 | 66.36 | 416.61 |

TABLE 2

Analysis of antibody binding to CD3 negative cells (MOLT-4) by FACS. The median fluorescence intensity is shown for each concentration tested.

| | | | | | | |
|---|---|---|---|---|---|---|
| Concentration (nM) | Rit-v1a-201 | Rit-v1a-145-204 | Rit-v1a-145-PF01 | Rit-v1a-145-PF02 | Rituximab | 200 |
| 1 | 75.76 | 78.38 | 79.32 | 73.79 | 78.96 | 79.40 |
| 10 | 72.18 | 75.93 | 81.42 | 72.70 | 75.92 | 76.59 |
| 100 | 61.51 | 62.37 | 79.01 | 70.44 | 68.42 | 70.49 |
| 1000 | 62.96 | 73.90 | 59.89 | 61.81 | 67.39 | 61.37 |

### Example 196. FcRn Binding Analysis

Binding to the FcRn receptor was determined at pH 7.4 and pH 6.0 using a Biacore T200 (serial no. 1909913) using single-cycle kinetics and running Biacore T200 Evaluation Software V 2.0.1. A CM5 chip was coupled with FcRn in sodium acetate pH 5.5 using standard amine chemistry. Serial dilution of bispecifics and controls were measured in PBS pH 7.4 with 0.05% tween-20 (9 points; 2-fold dilution series; 8000 nM Top conc.) and in PBS pH 6.0 with 0.05% tween-20 (3 points; 2-fold dilution series; 4000 nM Top conc.). A flow rate of 30 µl/min was used and an association time of 40 seconds and dissociation time of 75 seconds. Steady state analysis was used to analyze samples. FcRn binding was observed for all bispecifics at pH 6.0, with no binding observed at pH 7.4 (Table 3)

TABLE 3

Binding of different bispecifics, intermediates and control antibodies to FcRn at pH 6.0 or pH 7.4 as determined by Biacore.

| Antibody | pH | K_{D} (M) | R_{MAX} (RU) | Chi² (RU²) |
|---|---|---|---|---|
| Irrelevant IgG1 WT | 6.0 | 1.74E-06 | 67 | 0.783 |
| | 7.4 | - | - | - |
| Rituximab | 6.0 | 1.57E-06 | 96.4 | 2.82 |
| | 7.4 | - | - | - |
| Rit-v1a-201 | 6.0 | 2.16E-06 | 149.6 | 8.53 |
| | 7.4 | - | - | - |
| Rit-v1a-145-204 | 6.0 | 1.91E-06 | 122.9 | 5.36 |
| | 7.4 | - | - | - |
| Rit-v1a-145-PF01 | 6.0 | 1.90E-06 | 114.6 | 4.02 |
| | 7.4 | - | - | - |
| Rit-v1a-145-PF02 | 6.0 | 2.05E-06 | 123.5 | 5.47 |
| | 7.4 | - | - | - |
| Rit-v1a-145 | 6.0 | 1.89E-06 | 89.8 | 2.01 |
| | 7.4 | - | - | - |

### Example 197 Effect of Bispecifics on Raji-B Tumor Cell Killing With Human PBMCs

Duplicate wells were plated with Raji-B cells (5e4 cells) and human PBMCs (5e5) (1:10 cell ratio) into 96 well plates. Serial dilution of bispecifics (1:10dilution; 8 points; 10 nM Top conc.) were added to wells and incubated for 24 hours at 37° C. in tissue culture incubator. Samples were stained with CD19, CD20 antibodies and propidium iodide was added prior to acquisition of BD Fortessa Cell Analyzer. Live RajiB cells were quantitated based on Pl-/CD19+/CD20+ staining via flow cytometry analysis. The percentage of live RajiB cells was calculated relative to untreated cells. Target-dependent cell killing was demonstrated both for bispecifics based on hOKT3 200 (FIG. 28) and for bispecifics based on anti-4-1BB PF31 (FIG. 29).

### Example 198. Effect of Bispecifics on Cytokine Secretion in a Co-Culture of Raji-B Tumor Cells And Human PBMCs.

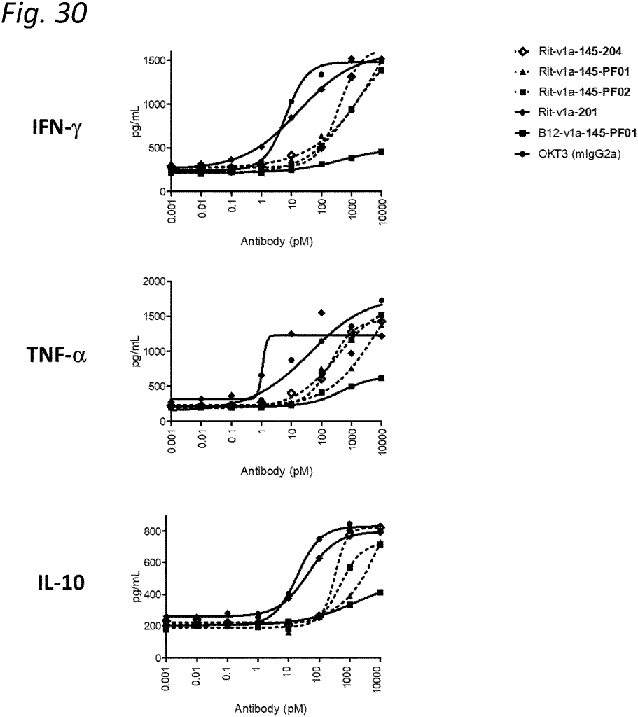

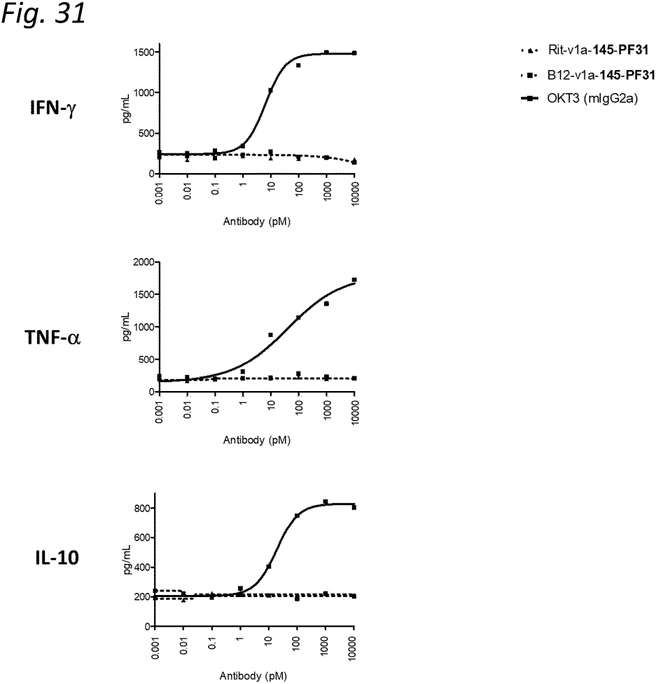

Duplicate wells were plated with Raji-B cells (5e4 cells) and human PBMCs (5e5) (1:10 cell ratio) into 96 well plates. Serial dilution of bispecifics (1:10 dilution; 8 points; 10 nM Top conc.) were added to wells and incubated for 24 hours at 37° C. in tissue culture incubator. Cytokine analysis was conducted on the supernatant for TNF-α, IFN-y and IL-10 (Kit: HCYTOMAG-60K-05, Merck Millipore). FIG. 30 shows cytokine levels for bispecifics based on hOKT3 200 and FIG. 31 shows cytokine levels for bispecifics based on anti-4-1BB PF31.

### SEQUENCE LIST

Sequence identification of C-terminal sortase A recognition sequence (SEQ. ID NO: 1):

GGGGSGGGGSLPETGGHHHHHHHHHH

Sequence identification of sortase A (SEQ. ID NO: 2):

TGSHHHHHHGSKPHIDNYLHDKDKDEKIEQYDKNVKEQASKDKKQQAKPQ
IPKDKSKVAGYIEIPDADIKEPVYPGPATPEQLNRGVSFAEENESLDDQN
ISIAGHTFIDRPNYQFTNLKAAKKGSMVYFKVGNETRKYKMTSIRDVKPT
DVGVLDEQKGKDKQLTLITCDDYNEKTGVWEKRKIFVATEVK

Sequence identification of His6-TEVsite-GGG-IL 15Rα-IL 15 (SEQ. ID NO: 3):

MGSSHHHHHHSSGENLYFQGGGITCPPPMSVEHADIVWKSYSLYSRERYI
CNSGFKRKAGTSSLTECVLNKATNVAHWTTPSLKCIRDPALVHQRPAPPS
GGSGGGGSGGGSGGGGSLQNWVNVISDLKKIEDLIQSMHIDATLYTESDV
HPSCKVTAMKCFLLELQVISLESGDASIHDTVENLIILANNSLSSNGNVT
ESGCKECEELEEKNIKEFLQSFVHIVQMFINTS

Sequence identification of anti-4-1BB PF31 (SEQ. ID NO: 4):

DIVMTQSPPTLSLSPGERVTLSCRASQSISDYLHWYQQKPGQSPRLLIKY
ASQSISGIPARFSGSGSGTDFTLTISSLEPEDFAVYYCQDGHSFPPTFGG
GTKVEIKGGGGSGGGGSGGGGSGGGGSQVQLVQSGAEVKKPGASVKVSCK
ASGYTFSSYWMHWVRQAPGQRLEWMGEINPGNGHTNYSQKFQGRVTITVD
KSASTAYMELSSLRSEDTAVYYCARSFTTARAFAYWGQGTLVTVSSGGGG
SGGGGSLPETGGHHHHHH

Sequence identification of SYR-(G₄S)₃-IL15 (PF18) (SEQ. ID NO: 5):

SYRGGGGSGGGGSGGGGSNWVNVISDLKKIEDLIQSMHIDATLYTESDVH
PSCKVTAMKCFLLELQVISLESGDASIHDTVENLlILANNSLSSNGNVTE
SGCKECEELEEKNIKEFLQSFVHIVQMFINTS

Sequence identification of SYR-(G₄S)₃-IL15Rα-linker-IL15 (PF26) (SEQ. ID NO: 6):

SYRGGGGSGGGGSGGGGSITCPPPMSVEHADIWVKSYSLYSRERYICNSG
FKRKAGTSSLTECVLNKATNVAHWTTPSLKCIRDPALVHQRPAPPSGGSG
GGGSGGGSGGGGSLQNWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTS

1. An antibody-payload conjugate having structure (1): 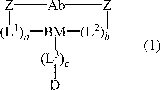
wherein:
Ab is an antibody;
a, b and c are each independently 0 or 1;
L¹, L², and L³ are linkers;
D is a payload;
BM is a branching moiety;
Z are connecting groups obtainable by a cycloaddition reaction.

2. The antibody-payload conjugate according to claim 1, wherein Z can be obtained by a [4+2] cycloaddition or a 1,3-dipolar cycloaddition.

3. The antibody-payload conjugate according to claim 1, wherein Z contains a triazole, a cyclohexene, a cyclohexadiene, an isoxazoline, an isoxazolidine, a pyrazoline, a piperazine.

4. The antibody-payload conjugate according to claim 1, wherein each of L¹, L², and L³ if present, are a chain of at least 2 atoms selected from C, N, O, S and P.

5. The antibody-payload conjugate according to claim 1, which has structure (5): 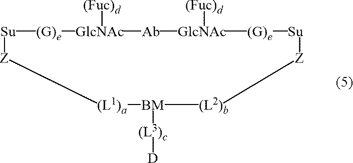
wherein:
e is an integer in the range of 010;
Su is a monosaccharide;
G is a monosaccharide moiety;
GlcNAc is an N-acetylglucosamine moiety;
Fuc is a fucose moiety;
d is 0 or 1.

6. The antibody-payload conjugate according to claim 1, wherein a and b are 1.

7. The antibody-payload conjugate according to claim 6, wherein L¹ and L² are the same.

8. The antibody-payload conjugate according to claim 5, wherein each occurrence of Su, Z, G and e are also the same.

9. The antibody-payload conjugate according to claim 1, wherein branching moiety BM is selected from a carbon atom, a nitrogen atom, a phosphorus atom, a (hetero)aromatic ring, a (hetero)cycle or a polycyclic moiety.

10. The antibody-payload conjugate according to claim 1, wherein L³ is -(L⁴)ₙ-(L⁵)ₒ-(L⁶)ₚ-(L⁷)_{q}-, wherein L⁴, L⁵, L⁶ and L⁷ are linkers that together form linker L⁵, n, o, p and q are individually 0 or 1.

11. The antibody-payload conjugate according to claim 10, wherein:
(a) linker L^{d} is represented by -(W)ₖ₁-(A)_{d1}-(B)ₑ₁-(A)_{f1}-(B)_{g1}—C(O)—, wherein:
d1 = 0 or 1;
e1 = an integer in the range 110;
f1 = 0, or 1;
g1 = an integer in the range 010;
k1 = 0 or 1 with the proviso that if k1 = 1 then d1 = 0;
A is a sulfamide group according to structure (23) 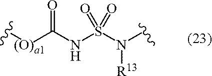
wherein a1 = 0 or 1, and R
¹² is selected from the group consisting of hydrogen, C₁ - C₂₄ alkyl groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups, the C₁ - C₂₄ alkyl groups, C₃ - C₂₄ cycloalkyl groups, C₂ - C₂₄ (hetero)aryl groups, C₃ - C₂₄ alkyl(hetero)aryl groups and C₃ - C₂₄ (hetero)arylalkyl groups optionally substituted and optionally interrupted by one or more heteroatoms selected from O, S and NR¹⁴ wherein R¹⁴ is independently selected from the group consisting of hydrogen and C₁ — C₄ alkyl groups, or R¹³ is D connected to N, possibly via a spacer moiety;
B is a —CH₂—CH₂—O— or a —O—CH₂—CH₂— moiety, or (B)ₑ₁ is a —(CH₂—CH₂—O)ₑ₃—CH₂—CH₂— moiety, wherein e3 is defined the same way as e1;
W is —OC(O)—, —C(O)O—, —C(O)NH—, -NHC(O)-, —OC(O)NH—, -NHC(O)O-, C(O)(CH₂)mC(O)—, —C(O)(CHz)mC(O)NH— or -(4-Ph)CH₂NHC(O)(CH₂)mC(O)NH-, wherein m is an integer in the range 0 - 10; and/or
(b) linker L⁵ is a peptide spacer; and/or
(c) linker L⁶ is a self-immolative spacer; and/or
(d) linker L⁷ is an aminoalkanoic acid spacer according to the structure —N—(Cₓ—alkylene)—C(O)—, wherein x is an integer in the range 1 - 10; or
linker L⁷ is a an ethyleneglycol spacer according to the structure -N-(CH₂-CH₂-O)ₑ₆-(CH₂)ₑ₇—C(O)—, wherein e6 is an integer in the range 1 - 10 and e7 is an integer in the range 1 - 3.

12. The antibody-payload conjugate according to claim 11, wherein L⁵ is represented by general structure (27): 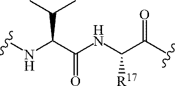
wherein, R
¹⁷ = CH₃ or CH₂CH₂CH₂NHC(O)NH₂.

13. The antibody-payload conjugate according to claim 11, wherein L⁶ is a para-aminobenzyloxycarbonyl (PABC) derivative according to structure (25) 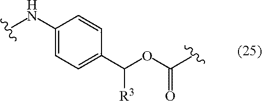
wherein R
³ is H, R⁴ or C(O)R⁴, wherein R⁴ is C₁ - C₂₄ (hetero)alkyl groups, C₃ - C₁₀ (hetero)cycloalkyl groups, C₂ - C₁₀ (hetero)aryl groups, C₃ - C₁₀ alkyl(hetero)aryl groups and C₃ - C₁₀ (hetero)arylalkyl groups, which are optionally substituted and optionally interrupted by one or more heteroatoms selected from O, S and NR⁵ wherein R⁵ is independently selected from the group consisting of hydrogen and C₁ — C₄ alkyl groups.

14. The antibody-payload conjugate according to claim 1, wherein D is a cytotoxin selected from PBD dimers, indolinobenzodiazepine dimers (IGN), enediynes, PNU159,682, duocarmycin dimers, amanitin and auristatins.

15. A method for preparing an antibody-payload conjugate having a hypothetical payload-to-antibody ratio of 1, comprising:
(a) reacting a compound having structure (2) containing at least two reactive groups Q with an antibody having structure (3), which is symmetrically functionalized with two reactive groups F: 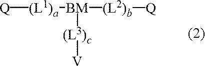 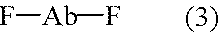
wherein:
Ab is an antibody;
a, b and c are each individually 0 or 1;
L¹, L², and L³ are linkers;
V is a reactive group Q′ or a payload D;
BM is a branching moiety;
Q and F are reactive groups capable of undergoing a cycloaddition reaction, wherein they are joined in connecting group Z;
to obtain a functionalized antibody according to structure (1): 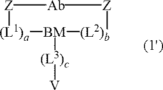
wherein Z is a connecting group obtained by the cycloaddition reaction of Q with F; wherein the functionalized antibody according to structure (1′) is the antibody-payload conjugate in case V is the payload D; or the functionalized antibody according to structure (1′) is further reacted according to step (b) in case V is a reactive group Q′;
(b) in case V = Q′, reacting reactive group Q′ with a payload containing a reactive group F′ to obtain the antibody-payload conjugate wherein V is the payload D.

16. The method according to claim 15, wherein the cycloaddition reaction is a [4+2] cycloaddition or a 1,3-dipolar cycloaddition.

17. The method according to claim 15, wherein Q comprises a terminal alkyne or a cyclooctyne moiety, preferably bicyclononyne (BCN), azadibenzocyclooctyne (DIBAC/DBCO), dibenzocyclooctyne (DIBO) or sulfonylated dibenzocyclooctyne (s-DIBO).

18. The method according to claim 15, wherein in step (a) a functionalized antibody according to structure (1) is obtained wherein D is the payload, and step (b) is not performed.

19. The method according to claim 15, wherein in step (a) a functionalized antibody according to structure (1) is obtained wherein D is a reactive group Q, and step (b) is performed.

20. A compound having structure (2): 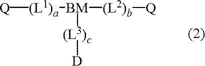
wherein:
a, b and c are each individually 0 or 1;
L¹, L² and L³ are linkers;
D is a payload;
BM is a branching moiety;
Q comprises a cyclooctyne moiety.

21. The compound according to claim 20, wherein Q is bicyclononyne (BCN), azadibenzocyclooctyne (DIBAC/DBCO), dibenzocyclooctyne (DIBO) or sulfonylated dibenzocyclooctyne (s-DIBO).

22. The compound according to claim 20, wherein D is a cytotoxin.

23. The compound according to claim 20, wherein and L¹ and L² are both present and identical.

24. The compound according to claim 20, wherein a = b = c = 1.

25. Pharmaceutical composition comprising the antibody-payload conjugate according to claim 1 and pharmaceutically acceptable carrier.

26. A method of treating cancer in a subject, comprising administering to the subject a composition according to claim 25.


SEQUENCE LISTING (6 sequences)

SEQ ID NO: 1 - protein, 26 aa, synthetic construct
  REGION 1..26: note = Sequence identification of C-terminal sortase A recognition sequence
GGGGSGGGGSLPETGGHHHHHHHHHH

SEQ ID NO: 2 - protein, 192 aa, synthetic construct
  REGION 1..192: note = Sequence identification of sortase A
TGSHHHHHHGSKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 3 - protein, 233 aa, synthetic construct
  REGION 1..233: note = Sequence identification of His6-TEVsite-GGG-IL15R -IL15
MGSSHHHHHHSSGENLYFQGGGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 268 aa, synthetic construct
  REGION 1..268: note = Sequence identification of anti-4-1BB PF31
DIVMTQSPPTLSLSPGERVTLSCRASQSISDYLHWYQQKPGQSPRLLIKYASQSISGIPARFSGSGSGTDFTLTISSLEPEDFAVYYCQDGHSFPPTFGGGTKVEIKGGGGSGGGGSGGGGSGGGGSQVQLVQSGAEVKKPGASVKVSCKASGYTFSSYWMHWVRQAPGQRLEWMGEINPGNGHTNYSQKFQGRVTITVDKSASTAYMELSSLRSEDTAVYYCARSFTTARAFAYWGQGTLVTVSSGGGGSGGGGSLPETGGHHHHHH

SEQ ID NO: 5 - protein, 132 aa, synthetic construct
  REGION 1..132: note = Sequence identification of SYR-(G4S)3-IL15 (PF18)
SYRGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 229 aa, synthetic construct
  REGION 1..229: note = Sequence identification of SYR-(G4S)3-IL15R?-linker-IL15 (PF26)
SYRGGGGSGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS